(19) United States
(12) Patent Application Publication
(10) Pub. No.: US 2018/0098977 A1
(43) Pub. Date: Apr. 12, 2018

(54) SCALABLE MICROPARTICULATE FORMULATIONS CONTAINING POLYMORPHIC NIMODIPINE FORM 2 PREPARED BY A SOLVENT EVAPORATION PROCESS

(71) Applicant: Edge Therapeutics, Inc., Berkeley Heights, NJ (US)

(72) Inventor: Alpaslan Yaman, Parsippany, NJ (US)

(21) Appl. No.: 15/679,790

(22) Filed: Aug. 17, 2017

Related U.S. Application Data
(60) Provisional application No. 62/378,518, filed on Aug. 23, 2016.

(51) Int. Cl. A61K 31/4422; A61K 9/00; A61K 9/14; A61K 9/19; A61P 9/00; A61P 25/00

(52) U.S. Cl. CPC A61K 31/4422; A61K 9/0019; A61P 25/00; A61K 9/19; A61P 9/00; A61K 9/146

(57) ABSTRACT

The described invention provides stable sustained release particulate formulations of polymorphic Form II of nimodipine and processes for their manufacture that not only can control formation of nimodipine polymorphs, but are practical, consistent from batch to batch, scalable, step-economical and efficient.

### CROSS-REFERENCE TO RELATED APPLICATIONS

This application claims the benefit of priority to U.S. Provisional Application No. 62/378,518 filed on Aug. 23, 2016, the entire contents of which are hereby incorporated by reference.

### FIELD

The described invention relates to manufacture and scale-up of microparticulate formulations of polymorphic form II of the dihydropyridine L-type calcium channel antagonist nimodipine.

### BACKGROUND OF THE INVENTION

DCI is a multifactorial process due to at least three processes, as well as to early brain injury. Angiographic vasospasm is one process that contributes to DCI. Other processes that may contribute to DCI are cortical spreading ischemia and formation of microthromboemboli. While conventional therapies have been focusing on treating cerebral vasospasms following subarachnoid hemorrhage, accumulating evidence suggests that these additional complications derived from subarachnoid hemorrhage need to be targeted for treatment interventions in order to improve prognosis. Cortical spreading ischemia, which was described in animal models of SAH as a novel mechanism that may cause DCI, has been detected in humans with SAH and angiographic vasospasm.

Each year, about 1 in 10,000 people have an aneurysm rupture. Mortality and morbidity increase with the volume of hemorrhage and reflect the age and health status of the patient, with the chance of developing an aneurysm increasing steadily with age. Rebleeding is exceptionally adverse due to the increase in volume of SAH as well as the increased likelihood of extension into the brain and ventricles. Most deaths resulting from aneurysmal rupture occur outside of hospitals or shortly after admission due to the effects of the initial bleed or early rebleeding. Potential manifestation of symptoms from vasospasm occurs only in those patients who survive past the first few days. The incidence of vasospasm is less than the incidence of SAH (since only some patients with SAH develop vasospasm). The incidence of vasospasm will depend on the type of patient a given hospital receives and the methods by which vasospasm is diagnosed.

Nimodipine has been shown in clinical trials to reduce the chance of a poor outcome, however it may not significantly reduce the amount of vasospasm detected on angiography. Other calcium channel antagonists and magnesium sulfate have been studied, but are not presently recommended. There is no evidence that shows benefit if nimodipine is given intravenously. In traumatic SAH, the efficacy of oral nimodipine remains in question.

Hemodynamic manipulation, previously referred to as “triple H” therapy, often is used as a measure to treat vasospasm. This entails the use of intravenous fluids to achieve a state of hypertension (high blood pressure), hypervolemia (excess fluid in the circulation) and hemodilution (mild dilution of the blood). Induced hypertension is believed to be the most important component of this treatment although evidence for the use of this approach is inconclusive, and no sufficiently large randomized controlled trials ever have been undertaken to demonstrate its benefits.

If symptomatic vasospasm is resistant to medical treatment, angiography may be attempted to identify the sites of vasospasm and to administer vasodilator medication (drugs that relax the blood vessel wall) directly into the artery (pharmacological angioplasty), and mechanical angioplasty (opening the constricted area with a balloon) may be performed.

For over 35 years, physicians have been trying to prevent or reduce the incidence of adverse consequences of SAH, and have had limited effect due to side effects of current agents or lack of efficacy. There currently are no FDA approved agents for the reduction of delayed ischemic neurologic deficits also known as delayed cerebral ischemia (DCI). Current methods to prevent vasospasm have failed due to lack of efficacy or to safety issues, primarily hypotension and cerebral edema. Currently, the only FDA-approved available agent is nimodipine, which does not reduce vasospasm, although it improved outcome in SAH patients.

Voltage-gated calcium channel antagonists may be effective in preventing and reversing vasospasm to a certain extent, however, prior art treatments administer doses too low to exert a maximal pharmacologic effect. Without being limited by theory, it is postulated that the systemic delivery of the voltage-gated calcium channel antagonists may cause side effects that mitigate the beneficial effects on vasospasm, such as, for example, systemic hypotension and pulmonary vasodilation with pulmonary edema, which prevent the administration of higher systemic doses. Dilation of blood vessels in the lungs also may cause lung edema and lung injury.

A microparticulate formulation of nimodipine that, when administered intraventricularly or intracisternally, enables localized delivery from the site of delivery into the cerebrospinal fluid in the subarachnoid space so that the therapeutic agent flows around the cerebral arteries in the subarachnoid space without entering systemic circulation in an amount to cause unwanted side effects, has been described.

U.S. Pat. Nos. 8,821,944 and 9,399,019 describe nimodipine microparticles prepared at laboratory scale by an oil/water emulsion process and dried in an agitated filter dryer under nitrogen flow. Up to three drug forms, in varying ratios, were present in the microparticle lots after processing: crystalline form I, crystalline form II, and amorphous nimodipine. Crystalline form II and the amorphous component caused aggregation of the product prepared by this process, leading to poor product performance. A GMP microparticulate formulation containing a crystalline polymorphic form I of nimodipine characterized by a plurality of microparticles, dispersal of the polymorphic form I of nimodipine throughout each microparticle, at least 70% by weight relative to the total weight of nimodipine of form I of nimodipine, and a pharmaceutically acceptable carrier, was prepared by a single emulsion process with suspended drug in an ethyl acetate polymer solution. The dispersed phase consisted of a 20% polymer solution in ethyl acetate with nimodipine added directly to the polymer solution to form a suspension. The continuous phase comprised a continuous process medium comprising 2% polyvinyl alcohol solution saturated with 3% ethyl acetate. A FormEZE™ column packed with 500 um beads was used to form the emulsion. The dispersed phase and continuous phase were added at a rate of 20 mL/min and 40 mL/min. respectively. The emulsified particles were extracted into water that was added at a rate of 1500 mL/min. The particles were collected over 125 and 25 μm sieves and then dried under nitrogen flow. The delivery system is characterized by delayed release of the polymorphic form I of nimodipine from the delivery system such that one half of the polymorphic form I of nimodipine is released within 1 day to 30 days in vivo. This product candidate is manufacturable into a drug product, exhibits the targeted product profile of the EG-1962 drug candidate at the particular time in development with respect to sustained release, and is stable for up to 24 months at frozen and refrigerated storage conditions. Tested batches of this formulation contain greater than 70% form I nimodipine, determined on an API basis.

NEWTON (Nimodipine microparticles to Enhance recovery While reducing TOxicity after subarachNoid hemorrhage) was a multicenter, randomized, controlled, open-label Phase 1/2 study evaluating the safety, tolerability and pharmacokinetics of escalating doses of a polymeric nimodipine microparticle suspended in a diluent of hyaluronic acid (EG-1962) compared to the current standard of care, oral nimodipine, in subjects with an aneurysmal subarachnoid hemorrhage (aSAH).

Fifty-four patients were randomized to receive EG-1962 and 18 patients were randomized to receive oral nimodipine. Pooled efficacy results of the NEWTON study showed that 60 percent of patients treated with EG-1962 achieved a favorable outcome (scores of 6-8 as measured by the Extended Glasgow Outcome Score [GOSE]) at 90 days compared to 28 percent of patients in the active control standard of care oral nimodipine arm who achieved a favorable outcome. In addition, improved efficacy was supported by a reduction in vasospasm, delayed cerebral ischemia and use of rescue therapies.

The primary endpoint was to establish the maximum tolerated dose, which has been determined to be 800 mg. Safety results showed that no patients (0 of 54) experienced EG-1962-related hypotension, while 17 percent of patients (three of 18) treated with oral nimodipine experienced drug-related hypotension. The secondary endpoint of characterizing the pharmacokinetics of EG-1962 was also met. The steady-state plasma concentration measured in patients treated with EG-1962 was below 30 ng/ml, the level of plasma concentration observed to cause systemic hypotension.

The design and development of long-acting or sustained-release delivery formulations have been the focus of considerable efforts in the pharmaceutical industry for decades.

Active pharmaceutical ingredients (APIs) are often administered to patients in their solid-states. Molecular solids or solid phases have been defined in thermodynamic terms as states of matter that are uniform in chemical composition and physical state. Molecular solids can exist in crystalline or noncrystalline (amorphous) phases depending on the extent of their three-dimensional order and relative thermodynamic stability. Crystalline states are characterized by a periodic array of molecules within a three-dimensional framework, termed a lattice, which are influenced by intra- and intermolecular interactions. Crystalline forms may also include hydrates and/or solvates of the same compound.

A given crystalline form of a particular API often constitutes an important determinant of the API's ease of preparation, hygroscopicity, stability, solubility, shelf-life, ease of formulation, rate of dissolution in the gastrointestinal tract and other fluids, and in vivo bioavailability. Choice of a crystalline form will depend on a comparison of physical property variables of the different forms. In certain circumstances, one form may be preferred for ease of preparation and stability leading to longer shelf-lives. In other cases, an alternate form may be preferred for higher dissolution rate and/or better bioavailability.

Polymorphism refers to the ability of a molecule to exist in two or more crystalline forms in which the molecules within a crystal lattice may differ in structural arrangement (packing polymorphism) and/or in conformation (conformational polymorphism). Polymorphic structures have the same chemical composition but different lattice structures and/or conformations resulting in different thermodynamic and kinetic properties. Thus, in the solid phase, polymorphic forms of an API exhibit different physical, chemical and pharmacological properties, such as in solubility, stability, melting point, density, bioavailability, X-ray diffraction patterns, molecular spectra, etc. However, in liquid or gaseous phases, polymorphic forms lose their structural organization and hence have identical properties. Phase transitions from one form to another may be reversible or irreversible. Polymorphic forms that are able to transform to another form without passing through a liquid or gaseous phase, are known as enantiotropic polymorphs, whereas those that are unable to interconvert under these conditions, are monotropic.

Enantiomers of chiral APIs may crystallize in three forms: (1) a racemate form in which the crystal lattice contains a regular arrangement of both enantiomers in equal amounts; (2) enantiopure forms in which the crystal lattice contains a regular arrangement of one enantiomer and not the other and vice versa; and (3) a conglomerate form in which there is a 1:1 physical mixture of two crystal lattices, one made up of a regular arrangement of one enantiomer and the other a regular arrangement of the other enantiomer.

Nimodipine [isopropyl(2-methoxyethyl)-1,4-dihydro-2,6-dimethyl-4-(3-nitrophenyl)-3,5-pyridinedicarboxylate] is a member of the dihydropyridine class of drugs belonging to the calcium channel antagonist family of pharmaceutical agents. The two forms of Nimodipine are presented below: on the left is the non-ionized form, and on the right is the ionized form:

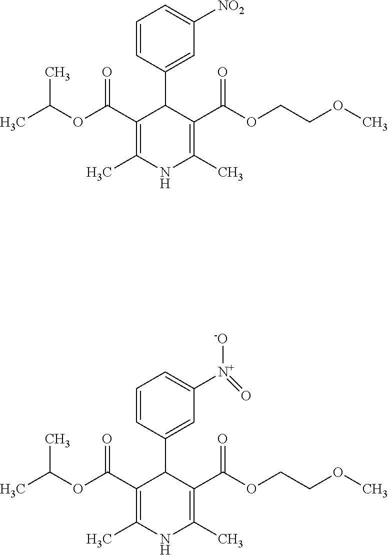

Nimodipine can exist in amorphous or crystalline forms depending on treatment and storage conditions. It exists as two polymorphic forms in the solid state. Modification I is a yellow to dark yellow colored compound, that melts at +124±1° C. and crystallizes as the racemic compound (Racemic Nimodipine Form I); commercially available nimodipine exists primarily as Form I. Modification II is a very pale yellow to almost white colored compound that melts at +116±1° C. and is a conglomerate (Conglomerate, Form II). Form II, the conglomerate form, is a 1:1 mixture of two crystal lattices, one containing one enantiomer and the other containing the opposite enantiomer (U.S. Pat. No. 5,599,824, incorporated herein by reference; Grunenberg, A. et al., “Polymorphism in binary mixtures, as exemplified by nimodipine”, International Journal of Pharmaceutics, (1995), 118: 11-21; Grunenberg, A. et al., “Theoretical derivation and practical application of energy/temperature diagrams as an instrument in preformulation studies of polymorphic drug substances”, International Journal of Pharmaceutics, (1996), 129: 147-158; Docoslis, A. et al., “Characterization of the distribution, polymorphism, and stability of nimodipine in its solid dispersions in polyethylene glycol by micro-Raman spectroscopy and powder X-ray diffraction”, The AAPS Journal, 2007, 9(3): Article 43). Form II is the thermodynamically stable form between absolute zero and about 90° C., where thermodynamic stability refers to stability of the crystal state and the potential to interconvert between polymorphic forms. Accordingly, the most stable form of nimodipine at room temperature is Form II. Below 90° C., nimodipine is in a metastable form, and the rate of conversion from Form II to Form I is determined by temperature and incentives to change form. At a temperature of greater than 90° C., Form II spontaneously converts to Form I, i.e., Form I is the more stable form at temperatures greater than 90° C.

Nimodipine has been indicated for use in neurological conditions such as aneurysms, subarachnoid hemorrhage, neuropathic pain, arthritis, etc. It is currently used in the U.S. to treat subarachnoid hemorrhage and migraine. Due to low solubility, nimodipine has been formulated as oral soft-gels, each capsule containing a 30 mg dose, commercially sold as Nimotop™, and, for use in patients incapable of swallowing, as an oral solution (commercially sold as Nymalize™, which contains 60 mg nimodipine per 20 mL, and the following inactive ingredients: ethanol, glycerin, methylparaben, polyethylene glycol, sodium phosphate monobasic, sodium phosphate dibasic, and water (http://www.rxlist.com/nymalize-drug.htm).

Despite its high permeability, oral administration of nimodipine is associated with low bioavailability. As nimodipine is a substrate for cytochrome P450 3A4 isoenzyme and the efflux pump P-glycoprotein (PgP), it is extensively and presystemically metabolized or expelled from cells, resulting in a relative bioavailability of approximately 18%. Thus, a relatively high dose and frequency regime is required. For oral dosing, due to limited stability, one or two 30 mg large-soft gel capsules are administered up to six times per day, which results in spikes of high plasma and cerebrospinal fluid (CSF) concentration with potential serious side-effects and is also a major inconvenience that leads to poor compliance. The resulting high dose nimodipine acts in a bolus-like manner whereby the plasma concentration spikes, often leading to hypotension. Also, the extreme peak to trough swing may result in a reflex increase in systolic flow velocities (PSV) or cerebral vasospasms, events that are prognostic of poor patient outcome.

In addition, as calcium channel antagonists, intravenous formulations of nimodipine cannot be used because of the high risk of inducing hypotension.

Various controlled release and combinatorial formulations of nimodipine, for example, for immediate release (within 0-12 hours of administration) or slower release (within 12-24 hours) of administration have been described. For example, [US Patent Publication No. US 2010/0215737 and 2010/0239665 describe an uncoated nimodipine minicapsule formulation made by adding appropriate quantities of micronized nimodipine, gelatin and sorbitol to water and heating to 80° C., continually stirring until a homogeneous solution is achieved. The solution is then processed into solid minispheres at an appropriate flow rate and vibrational frequency using the manufacturing processing method described in U.S. Pat. No. 5,882,680. The resulting minispheres are cooled in oil. The cooled minispheres are harvested and centrifuged to remove residual oil and dried overnight in an oven. The completed multiparticulate Nimodipine seamless minicapsules contained 37.5% w/w nimodipine, and had an average diameter in the range 1.50-1.80 mm. To prepare coated nimodipine minicapsules, some of the uncoated minicapsules are coated with Surelease® (e.g., 7.5% wt gain) using standard bottom spray fluidized bed coating, as enabled using a Diosna Minilab, to provide a 12-hour or a 24-hour release profile. Typically curing occurs at 40° C. over 24 hours. In another case, the coating is a higher weight gain Surelease®, such as 30% wt gain Surelease®. The described modified release solid dosage product comprising a plurality of minicapsules or minispheres containing nimodipine release more than 40% of the nimodipine within 12 hours, and Tmax is reached within 6 hours. These formulations are intended for sachet format, suppository format for vaginal or rectal administration, or a format for buccal or sublingual administration.

An orally administered immediate release formulation containing a co-precipitate of essentially amorphous nimodipine with poly-vinyl-pyrrolidone (PVP) is described in U.S. Pat. No. 5,491,154. A pharmaceutical preparation containing a suspension of a mixture of nimodipine Form II crystals in a suspension solution is described in U.S. Pat. No. 5,599,824. A solid dispersion of nimodipine Form II in PVP with fast release kinetics is described in Papageorgiou, G. Z. et al., “The effect of physical state on the drug dissolution rate: Miscibility studies of nimodipine with PVP”, Journal of Thermal Analysis and calorimetry, 2009, 95(3): 903-915.

To formulate a drug product, it is necessary that the product remain stable during drug development and that it is reproducibly manufacturable from small laboratory scale lots to commercial scale. A drug product is considered unstable when the drug substance/active ingredient loses sufficient potency to adversely affect the safety or efficacy of the drug or falls outside labeled specifications as shown by stability-indicating methods. To properly evaluate the stability of a drug product, the storage conditions under which the drug strength can be maintained in order to provide a safe and efficacious drug product are determined.

Particle size may affect bioavailability, content uniformity, suspension properties, solubility and stability. Crystal properties and the formation of different polymorphic drug forms in a microparticle may impact solubility, bioavailability, stability and overall product performance. Performance, in turn, can be considered as an indicator of the delivery of a drug from the dose form to the target site and depends upon the type of dose form and the route of administration. Suitable limits for key parameters affecting bioavailability need to be derived from batches of product showing acceptable in vivo performance.

During the development stage, a manufacturer gains information about the behavior and the physical and chemical properties of the drug substance, the composition of the product in terms of active ingredient(s) and key excipients, and the manufacturing process in order to identify and define the critical steps in the manufacturing process. Information generated is then used to identify and evaluate critical pharmaceutical process parameters that may need to be examined and controlled to ensure batch to batch reproducibility. Such parameters will vary depending on the nature of the product, the composition, and the proposed method of manufacture. In order to define the critical parameters, it may be necessary to make deliberate changes to demonstrate the robustness of the process and define the limits of tolerance.

Once the particular method of manufacture, based on a consideration of the physical and chemical properties of the active ingredient, the key excipients, the choice of formulation and the impact of processing on the product quality and stability, is defined, data is generated on different scales as the manufacturing process is developed to provide adequate proof of the feasibility of the process at the production scale to ensure the consistent quality of the product in line with the approved specification. For example, data derived from laboratory scale batches assist in the evaluation and definition of critical product performance characteristics, and pilot batches provide data predictive of the production scale product.

The described invention provides process and formulation development with respect to microparticulate formulations of nimodipine for site-specific delivery to CNS sites of administration that not only can control formation of drug polymorphs, but is practical, consistent from batch to batch, scalable, step-economical and efficient.

### SUMMARY OF THE INVENTION

The described invention relates to manufacture and scale-up of microparticulate formulations of polymorphic form II of the dihydropyridine L-type calcium channel antagonist nimodipine.

According to one aspect, the described invention provides a pharmaceutical composition formulated for delivery by injection containing a microparticulate formulation comprising (a) a suspension of microparticles comprising a therapeutic amount of a substantially pure Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide) polymer matrix, and (b) a pharmaceutically acceptable carrier comprising an agent that affects viscosity of the microparticulate suspension, wherein the microparticulate suspension comprising the polymorphic Form II of nimodipine is light stable, the Polymorphic Form II of nimodipine is chemically stable, release profile is consistent from batch-to-batch, and particle size is controllable. According to one embodiment of the pharmaceutical composition, the microparticulate suspension comprises a plurality of microparticles; or the microparticles are of a uniform distribution of microparticle size; or the mean particle size (D50) of the microparticles ranges from 20 μm to 250 μm; or the concentration of the polymer ranges from about 14% to about 30%; or the lactide to glycolide ratio of the poly (lactide-co-glycolide) is 50:50; or inherent viscosity of the polymer is at least 0.16 dl/g; or molecular weight of the polymer is at least 28 kDa; or the polymorphic form II of nimodipine is dispersed throughout the polymer matrix; or the polymer matrix is impregnated with the polymorphic Form II of nimodipine; or percentage of nimodipine retained by the microparticles relative to the total amount available is about 95%; or the microparticulate suspension is characterized by a drug load of about 65% polymorphic Form II of nimodipine by weight relative to the total weight of the formulation. According to another embodiment, the polymorphic form II of nimodipine includes less than 20% by weight of any other physical forms of nimodipine; or the microparticulate formulation contains less than 10% polymorphic Form I of nimodipine; or the microparticulate formulation is substantially free of polymorphic Form I of nimodipine.

According to another aspect, the suspension of microparticles comprising a therapeutic amount of the milled polymorphic Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide)polymer matrix is prepared by a scalable process comprising: (a) providing an API starting material containing a substantially pure polymorphic Form I of nimodipine; (b) forming polymorphic Form II of nimodipine in situ by (i) adding the API starting material of (a) to a polymer solution, and (ii) creating a mixture of the polymorphic Form II of nimodipine and the polymer solution; (c) homogenizing the mixture of (b) to form a disperse phase comprising the nimodipine; (d) providing a continuous phase in which the dispersed phase will form an emulsion; (e) introducing the dispersed phase and continuous phase into a reactor vessel, the reactor vessel including a continuous process medium, and forming an emulsion of the dispersed phase in the continuous phase comprising the nimodipine; (f) causing the polymer to form microparticles containing polymorphic Form II of nimodipine; (g) transporting the emulsion from the reactor vessel to a solvent removal vessel and removing the solvent; and (h) formulating the nimodipine Form II-containing microparticles by: (i) maintaining a suspension of nimodipine Form II-containing microparticles in the continuous phase; and (ii) washing the nimodipine Form II-containing microparticles; and (i) drying the nimodipine Form II-containing microparticles. According to one embodiment of the process, the API starting material is milled or unmilled; the solvent comprises ethyl acetate; and the washing is conducted by (i) replacing the continuous phase with water by moving the suspension through a filter adapted to remove continuous phase and return the microparticles to a process vessel while maintaining the suspension; (ii) replacing the ethyl acetate with water by moving the suspension through a filter adapted to eliminate the ethyl acetate and return the microparticles to a process vessel while maintaining the microparticles in suspension; and removing the suspension of microparticles containing the bioactive agent and formulating medium from the process vessel; or the washing is conducted by moving the suspension through a hollow fiber filter. According to another embodiment, the drying is by lyophilization or by a vacuum dryer. According to another embodiment, the distribution of microparticle size is such that D10>20 μm, D50 is 70-80 μm, and D90 is <200 μm.

According to another aspect, the suspension of microparticles comprising a therapeutic amount of the polymorphic Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide)polymer matrix is prepared by a scalable process comprising: (1) preparing an API starting material containing a substantially pure polymorphic nimodipine Form II by: (a) synthesizing an API starting material containing substantially pure polymorphic Form II of nimodipine; or (b) crystallizing Form II of nimodipine from Form I by dissolving Form I of nimodipine in a first solvent and evaporating the first solvent to yield Form II; (2) completing the disperse phase by adding the API starting material of step (1) to a polymer solution, thereby creating a mixture of polymorphic Form II of nimodipine and the polymer solution in a second solvent; (3) homogenizing the continuous phase comprising polyvinyl alcohol (PVA) in water with the dispersed phase of step (2) to form an emulsion; (4) introducing a water stream continuously post-microparticle formation, causing the polymer to form nimodipine Form II-containing microparticles; (5) transporting the emulsion from the reactor vessel to a solvent removal vessel and removing the solvent; (6) formulating the Form II containing microparticles by (i) maintaining a suspension of the Form II containing microparticles in the continuous phase; and (ii) washing the Form II containing microparticles; and (7) drying the Form II containing microparticles. According to one embodiment, the process further comprises milling, micronizing or both the API starting material. According to another embodiment, the API starting material containing the substantially pure polymorphic form II of nimodipine is characterized by a distribution of particle size of D10>2μ, D50>7μ and D90<10 μm. According to another embodiment, (a) the first solvent is ethanol; (b) the second solvent is ethyl acetate; and (c) the washing is conducted by (i) replacing the continuous phase with water by moving the suspension through a filter adapted to remove continuous phase and return the microparticles to a process vessel while maintaining the suspension; (ii) replacing the ethyl acetate with water by moving the suspension through a filter adapted to eliminate the ethyl acetate and return the microparticles to a process vessel while maintaining the microparticles in suspension; and (iii) removing the suspension of microparticles containing the bioactive agent and formulating medium from the process vessel; or the washing is conducted by moving the suspension through a hollow fiber filter.

According to another aspect, the described invention provides a method for reducing severity or incidence of a delayed complication associated with a brain injury including interruption of a cerebral artery that deposits blood in a subarachnoid space, wherein the delayed complication is selected from the group consisting of a microthromboembolism, a delayed cerebral ischemia (DCI) caused by formation one or more of microthromboemboli, or cortical spreading ischemia (CSI) and a cortical spreading ischemia (CSI) comprising: a) providing the pharmaceutical composition according to claim 1, and (b) administering the pharmaceutical composition locally, either (i) intraventricularly; (ii) intracisternally into the subarachnoid space in a subarachnoid cistern; or (iii) intrathecally into the spinal subarachnoid space, wherein the therapeutic amount of the substantially pure polymorphic Form II of Nimodipine having an X-ray powder diffraction spectrum substantially the same as the X-ray powder diffraction (XRPD) spectrum shown in FIG. 14B, a melting point of 116±1° C. as measured by differential scanning calorimetry or both that contacts and flows around the at least one cerebral artery in the subarachnoid space is effective to improve cerebral perfusion and to treat the delayed complication without entering systemic circulation in an amount to cause unwanted side effects including systemic hypotension and pulmonary vasodilation with pulmonary edema.

### BRIEF DESCRIPTION OF THE DRAWINGS

FIG. 1 is a flow chart showing one embodiment of a microsphere manufacturing process according to the described invention.

FIG. 2A-FIG. 2B contains plots of nimodipine cumulative release (%) in vitro vs. time (days) of undissolved batches of (FIG. 2A) milled nimodipine; (FIG. 2B) unmilled nimodipine, showing in vitro release of undissolved nimodipine batches.

FIG. 3 is a plot of nimodipine cumulative release (%) in vitro vs. time (days) of undissolved batches of unmilled nimodipine, showing the effect of washing volume exchanges on in vitro release of undissolved nimodipine batches.

FIG. 4 is a plot of nimodipine cumulative release (%) in vitro vs. time (days) of undissolved batches of unmilled nimodipine, showing the effect of washing temperature and cycle on in vitro release of undissolved nimodipine batches.

FIG. 5 is a plot of nimodipine cumulative release (%) in vitro vs. time (days) of undissolved batches of unmilled nimodipine, showing the effect of hold time on in vitro release of undissolved nimodipine batches.

FIG. 6 shows the effect of temperature treatment on microparticle formation by light microscopy. Panels from left to right: first panel, just after microsphere (MS) formation; second panel, during annealing, 60 C; third panel, during annealing, 80 C, fourth panel, nonaggregated portion annealed at 95 C, t=15 min.

FIG. 7 is a plot of nimodipine cumulative release (%) in vitro vs. time (days) of undissolved batches of Form II of nimodipine.

FIG. 8 is a plot of nimodipine cumulative release (%) in vitro vs. time (days) of undissolved batches of Form II of nimodipine (5 g).

FIG. 9 is a plot of nimodipine cumulative release (%) in vitro vs. time (days) of undissolved batches showing the effect of DP mixing time on in vitro release (5 g).

FIG. 10A-FIG. 10D show light micrographs (bar=500 μm) showing formation of drug crystals with conversion of Form I to Form II nimodipine as a function of disperse phase mixing time. (FIG. 10A) Dispersed phase: 15 min. DP mixing time; (FIG. 10B) microspheres: 15 min. DP mixing time; (FIG. 10C) dispersed phase: 60 min DP mixing time; (FIG. 10D) microspheres: 60 min. DP mixing time.

FIG. 11 is a plot of nimodipine cumulative release (%) in vitro vs. time (days) of undissolved batches of Form II of nimodipine (50 g) showing the effect of dispersed phase mixing time on in vitro release.

FIG. 12 is a plot of nimodipine cumulative release (%) in vitro vs. time (days) of undissolved batches of Form II of nimodipine (5 g) showing the effect of scale-up on in vitro release.

FIG. 13 is a plot of nimodipine cumulative release (%) in vitro vs. time (days) of undissolved 50 g and 500 g batches of Form II of nimodipine lot CM021116.

FIG. 14A-FIG. 14C show X ray powder diffraction profiles. The SRPD pattern was collected with a PANalytical X′Pert PRO MPD diffractometer using an incident beam of Cu radiation produced using an Optix long, fine-focus source. An elliptically graded multilayer mirror was used to focus Cu Kα X-rays through the specimen and onto the detector. Prior to analysis, a silicon specimen (NIST SRM 640e) was analyzed to verify the observed position of the Si 111 peak is consistent with the NIST-certified position. A specimen of the sample was sandwiched between 3 μm-thick films and analyzed in transmission geometry. A beam-stop, short antiscatter extension, antiscatter knife edge were used to minimize the background generated by air. Soller slits for the incident and diffracted beams were used to minimize broadening from axial divergence. Diffraction pattern was collected using a scanning position-sensitive detector (X′Celerator) located 240 mm from the specimen and Data Collector software v. 2.2b. The data acquisition parameters for the pattern are displayed above the image including the divergence slit (DS) before the mirror. FIG. 14A shows a reference X-ray powder diffraction spectrum for Form I of nimodipine; FIG. 14B shows a reference X-ray powder diffraction spectrum for Form II of nimodipine; FIG. 14C shows an X-ray powder diffraction profile of an actual sample produced by the process whereby Form I is converted to Form II in situ. The results show that the sample is Form II with the absence of form I.

### DETAILED DESCRIPTION

### Glossary

The term “active” as used herein refers to the ingredient, component or constituent of the compositions of the present invention responsible for the intended therapeutic effect.

The term “active pharmaceutical ingredient” (API; or Drug Substance) as used herein refers to any substance or mixture of substances intended to be used in the manufacture of a drug product and that, when used in the production of a drug, becomes an active ingredient of the drug product. Such substances are intended to furnish pharmacological activity or other direct effect in the diagnosis, cure, mitigation, treatment or prevention of disease or to affect the structure and function of the body.

The term “API Starting Material” as used herein refers to a raw material or an API used in the production of an API and that is incorporated as a significant structural fragment into the structure of the API. API starting materials normally are of defined chemical properties and structure.

The term “additive effect”, as used herein, refers to a combined effect of two chemicals that is equal to the sum of the effect of each agent given alone.

“Admixture” or “blend” is used herein to refer to a physical combination of two or more different components.

The term “administering” as used herein includes in vivo administration, as well as administration directly to tissue ex vivo. Generally, compositions may be administered systemically (e.g., orally, buccally, parenterally, by inhalation or insufflation (i.e., through the mouth or through the nose), or rectally) in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “agent” as used herein refers generally to an active compound(s) that is/are contained in or on the formulation. “Agent” includes a single such compound and is also intended to include a plurality of such compounds.

The term “agonist” as used herein refers to a chemical substance capable of activating a receptor to induce a pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “angiographic vasospasm” as used herein refers to the reduction of vessel size that can be detected on angiographic exams, including, but not limited to, computed tomographic, magnetic resonance or catheter angiography, occurring in approximately 67% of patients following subarachnoid hemorrhage.

The term “antagonist” as used herein refers to a substance that interferes with the effects of another substance. Functional or physiological antagonism occurs when two substances produce opposite effects on the same physiological function. Chemical antagonism or inactivation is a reaction between two substances to neutralize their effects. Dispositional antagonism is the alteration of the disposition of a substance (its absorption, biotransformation, distribution, or excretion) so that less of the agent reaches the target or its persistence there is reduced. Antagonism at the receptor for a substance entails the blockade of the effect of an antagonist with an appropriate antagonist that competes for the same site.

The term “batch” as used herein refers to a specific quantity of a drug or other material produced in a process or series of processes so that it is expected to have uniform character and quality, within specified limits. The batch size can be defined either by a fixed quantity or by the amount produced in a fixed time interval.

The term “batch formula (composition)” as used herein refers to a complete list of the ingredients and their amounts to be used for the manufacture of a representative batch of the drug product.

The term “biocompatible” as used herein refers to a material that is generally non-toxic to the recipient and does not possess any significant untoward effects to the subject and, further, that any metabolites or degradation products of the material are non-toxic to the subject. Typically a substance that is “biocompatible” causes no clinically relevant tissue irritation, injury, toxic reaction, or immunological reaction to living tissue.

The term “biodegradable” as used herein refers to a material that will erode to soluble species or that will degrade under physiologic conditions to smaller units or chemical species that are, themselves, non-toxic (biocompatible) to the subject and capable of being metabolized, eliminated, or excreted by the subject.

The term “chiral” is used to describe asymmetric molecules that are nonsuperposable since they are mirror images of each other and therefore have the property of chirality. Such molecules are also called enantiomers and are characterized by optical activity.

The term “chirality” refers to the geometric property of a rigid object (or spatial arrangement of points or atoms) of being non-superposable on its mirror image; such an object has no symmetry elements of the second kind (a mirror plane, σ=51, a center of inversion, i=S2, a rotation-reflection axis, S2n). If the object is superposable on its mirror image, the object is described as being achiral.

The term “chirality axis” refers to an axis about which a set of ligands is held so that it results in a spatial arrangement which is not superposable on its mirror image. For example, with an allene abC═C═Ccd, the chiral axis is defined by the C═C═C bonds; and with an ortho-substituted biphenyl C-1, C-1′, C-4 and C-4′ lie on the chiral axis.

The term “chirality center” refers to an atom holding a set of ligands in a spatial arrangement, which is not superimposable on its mirror image. A chirality center may be considered a generalized extension of the concept of the asymmetric carbon atom to central atoms of any element.

The terms “chiroptic” or “chiroptical” refer to the optical techniques (using refraction, absorption or emission of anisotropic radiation) for investigating chiral substances (for example, measurements of optical rotation at a fixed wavelength, optical rotary dispersion (ORD), circular dichroism (CD) and circular polarization of luminescence (CPL)).

The term “chirotopic” refers to an atom (or point, group, face, etc. in a molecular model) that resides within a chiral environment. One that resides within an achiral environment has been called achirotopic.

The term “cistern” or “cisterna” as used herein means a cavity or enclosed space serving as a reservoir.

The term “complication” as used herein refers to a pathological process or event during a disorder that is not an essential part of the disease, although it may result from it or from independent causes. A delayed complication is one that occurs some time after a triggering effect. Complications associated with subarachnoid hemorrhage include, but are not limited to, delayed cortical ischemia due to angiographic vasospasm, microthromboemboli, cortical spreading ischemia or a combination thereof.

The term “contact” and all its grammatical forms as used herein refers to an instance of exposure by close physical contact of at least one substance to another substance.

The term “controlled release” is intended to refer to a drug-containing formulation in which the manner and profile of drug release from the formulation are regulated. This refers to immediate as well as non-immediate release formulations, with non-immediate release formulations including, but not limited to, sustained release and delayed release formulations.

The term “cortical spreading depolarization” or “CSD” as used herein refers to a wave of near-complete neuronal depolarization and neuronal swelling in the brain that is ignited when passive cation influx across the cellular membrane exceeds ATP-dependent sodium and calcium pump activity. The cation influx is followed by water influx and shrinkage of the extracellular space by about 70%. If normal ion homeostasis is not restored through additional recruitment of sodium and calcium pump activity, the cell swelling is maintained--, a process then termed “cytotoxic edema,” since it potentially leads to cell death through a protracted intracellular calcium surge and mitochondrial depolarization. CSD induces dilation of resistance vessels in healthy tissue; hence regional cerebral blood flow increases during the neuronal depolarization phase. (Dreier, J. P. et al., (2009) Brain 132: 1866-81).

The term “cortical spreading ischemia” or “CSI,” or “inverse hemodynamic response” refers to a severe microvascular spasm that is coupled to the neuronal depolarization phase. The resulting spreading perfusion deficit prolongs neuronal depolarization (as reflected by a prolonged negative shift of the extracellular direct current (DC) potential) and the intracellular sodium and calcium surge. The hypoperfusion is significant enough to produce a mismatch between neuronal energy demand and supply. (Id.).

As used herein, the terms “crystalline form” and “crystal form” are used interchangeably to mean that a certain material has definite shape and an orderly arrangement of structural units, which are arranged in fixed geometric patterns or lattices.

The term “delayed cerebral ischemia” or “DCI” as used herein refers to the occurrence of focal neurological impairment (such as hemiparesis, aphasia, apraxia, hem ianopia, or neglect), or a decrease in the Glasgow coma scale (either on the total score or on one of its individual components [eye, motor on either side, verbal]). This may or may not last for at least one hour, is not apparent immediately after aneurysm occlusion and cannot be attributed to other causes by means of clinical assessment, CT or magnetic resonance imaging (MRI) scanning of the brain, and appropriate laboratory studies. Angiographic cerebral vasospasm is a description of a radiological test (either CT angiography [CTA], MR angiography [MRA] MRA or catheter angiography [CA]), and may be a cause of DCI.

The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.”

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.” Derivatives can include chemical modifications, such as alkylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

The term “diastereoisomerism” refers to stereoisomerism other than enantiomerism. Diastereoisomers (or diastereomers) are stereoisomers not related as mirror images. Diastereoisomers are characterized by differences in physical properties, and by some differences in chemical behavior towards achiral as well as chiral reagents. Diastereomers have similar chemical properties, since they are members of the same family. Their chemical properties are not identical, however. Diastereomers have different physical properties: different melting points, boiling points, solubilities in a given solvent, densities, refractive indexes, and so on. Diastereomers also differ in specific rotation; they may have the same or opposite signs of rotation, or some may be inactive. The presence of two chiral centers can lead to the existence of as many as four stereoisomers. For compounds containing three chiral centers, there could be as many as eight stereoisomers; for compounds containing four chiral centers, there could be as many as sixteen stereoisomers, and so on. The maximum number of stereoisomers that can exist is equal to 2n, where n is the number of chiral centers. The term “diastereotopic” refers to constitutionally equivalent atoms or groups of a molecule which are not symmetry related. Replacement of one of two diastereotopic atoms or groups results in the formation of one of a pair of diastereoisomers. For example, the two hydrogen atoms of the methylene group

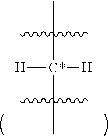

are diastereotopic.

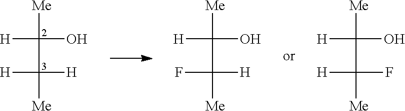 [SMILES: CCC(C)O ; CC(O)C(C)F ; CC(O)C(C)F]

The term “dissolution rate” as used herein refers to the amount of a drug that dissolves per unit time. The term “inherent dissolution rate” is the dissolution rate of a pure API under constant conditions of surface area, rotation speed, pH and ionic strength of the dissolution medium. Inherent dissolution rate is applicable to the determination of thermodynamic parameters associated with different crystalline phases and their solution-mediated phase transformations, investigation of the mass transfer phenomena during the dissolution process, determination of pH-dissolution rate preofiles and the evaluation of the impact of different pH values and the presence of surfactants on the solubilization of poorly soluble compounds. (Riekes, M. K. et al, “Development and Validation of an inherent dissolution method for nimodipine polymorphs,” Cent. Eur. J. Chem. (2014); 12(5): 549-56).

The term “dispersion”, as used herein, refers to a two-phase system, in which one phase is distributed as droplets in the second, or continuous phase. In these systems, the dispersed phase frequently is referred to as the discontinuous or internal phase, and the continuous phase is called the external phase and comprises a continuous process medium. For example, in course dispersions, the particle size is 0.5 μm. In colloidal dispersions, size of the dispersed particle is in the range of approximately 1 nm to 0.5 μm. A molecular dispersion is a dispersion in which the dispersed phase consists of individual molecules; if the molecules are less than colloidal size, the result is a true solution.

The term “disposed”, as used herein, refers to being placed, arranged or distributed in a particular fashion.

Dose-effect curves; The intensity of effect of a drug (y-axis) can be plotted as a function of the dose of drug administered (X-axis). (Goodman & Gilman's The Pharmacological Basis of Therapeutics, Ed. Joel G. Hardman, Lee E. Limbird, Eds., 10^{th} Ed., McGraw Hill, New York (2001), p. 25, 50). These plots are referred to as dose-effect curves. Such a curve can be resolved into simpler curves for each of its components. These concentration-effect relationships can be viewed as having four characteristic variables: potency, slope, maximal efficacy, and individual variation.

The location of the dose-effect curve along the concentration axis is an expression of the potency of a drug. Id. For example, if the drug is to be administered by transdermal absorption, a highly potent drug is required, since the capacity of the skin to absorb drugs is limited.

The slope of the dose-effect curve reflects the mechanism of action of a drug. The steepness of the curve dictates the range of doses useful for achieving a clinical effect.

The term “maximal or clinical efficacy” refers to the maximal effect that can be produced by a drug. Maximal efficacy is determined principally by the properties of the drug and its receptor-effector system and is reflected in the plateau of the curve. In clinical use, a drug's dosage may be limited by undesired effects.

Biological variability. An effect of varying intensity may occur in different individuals at a specified concentration or a drug. It follows that a range of concentrations may be required to produce an effect of specified intensity in all subjects.

Lastly, different individuals may vary in the magnitude of their response to the same concentration of a drug when the appropriate correction has been made for differences in potency, maximal efficacy and slope.

The duration of a drug's action is determined by the time period over which concentrations exceed the minimum effective concentration (MEC). Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the MEC for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. The therapeutic window reflects a concentration range that provides efficacy without unacceptable toxicity. Generally another dose of drug can be administered to maintain concentrations within the therapeutic window over time.

The term “drug substance” as used herein refers to an active ingredient intended to furnish pharmacological activity or other direct effect in the diagnosis, cure, mitigation, treatment or prevention of a disease, or to affect the structure and function of the body, but does not include intermediates used in synthesis of such ingredient.

The term “drug product” as used herein refers to a finished dosage form that contains a drug substance, generally, but not necessarily, in association with one or more other ingredients.

The term “effective amount” refers to the amount necessary or sufficient to realize a desired biologic effect.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “enantiomer” as used herein refers to one of a pair of optical isomers containing one or more asymmetric carbons (C*) whose molecular configurations have left- and right-hand (chiral) configurations. Enantiomers have identical physical properties, except as to the direction of rotation of the plane of polarized light. For example, glyceraldehyde and its mirror image have identical melting points, boiling points, densities, refractive indexes, and any other physical constant one might measure, except that they are non-superimposable and one rotates the plane-polarized light to the right, while the other to the left by the same amount of rotation.

The term “essentially the same” with reference to X-ray diffraction peak positions means that typical peak position and intensity variability are taken into account. For example, one skilled in the art will appreciate that the peak positions (28) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only.

The term “excipient” is used herein to include any other agent or compound that may be contained in a formulation that is not the bioactive agent. As such, an excipient should be pharmaceutically or biologically acceptable or relevant (for example, an excipient should generally be non-toxic to the subject). “Excipient” includes a single such compound and is also intended to include a plurality of such compounds.

The term “flowable”, as used herein, refers to that which is capable of movement in, or as if in, a stream by continuous change of relative position.

The term “formulation” as used herein refers to a listing of the ingredients and composition of the dosage form.

The term “hydrate” as used herein refers to a compound formed by the addition of water or its elements to another molecule. The water usually can split off by heating, yielding the anhydrous compound.

The term “hydrogel” as used herein refers to a substance resulting in a solid, semisolid, pseudoplastic, or plastic structure containing a necessary aqueous component to produce a gelatinous or jelly-like mass.

The term “hypertension” as used herein refers to high systemic blood pressure; transitory or sustained elevation of systemic blood pressure to a level likely to induce cardiovascular damage or other adverse consequences.

The term “hypotension” as used herein refers to subnormal systemic arterial blood pressure; reduced pressure or tension of any kind.

The term “impregnate”, as used herein in its various grammatical forms refers to causing to be infused or permeated throughout; to fill interstices with a substance.

The term “impurity” as used herein refers to any component present in the intermediate or API that is not the desired entity.

The term “impurity profile” as used herein refers to a description of the identified and unidentified impurities present in an API.

The terms “in-process control” or “process control” are used interchangeably to refer to checks performed during production to monitor and, if appropriate, to adjust the process and/or to ensure that the API conforms to its specifications.

The term “intermediate” as used herein refers to a material produced during steps of the processing of an API that undergoes further molecular change or purification before it becomes an API. Intermediates may or may not be isolated.

The terms “in the body”, “void volume”, “resection pocket”, “excavation”, “injection site”, “deposition site”, “implant site” or “site of delivery” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof.

The term “isolated molecule” as used herein refers to a molecule that is substantially pure and is free of other substances with which it is ordinarily found in nature or in vivo systems to an extent practical and appropriate for its intended use.

The term “isomer” as used herein refers to one of two or more molecules having the same number and kind of atoms and hence the same molecular weight, but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosphorus atoms; and allenic groups. Enantiomers are non-superimposable mirror images. A mixture of equal parts of the optical forms of a compound is known as a racemic mixture or racemate. Diastereomers are stereoisomers that are not mirror images. Stereoisomers may include enantiomers, diastereomers, or E or Z alkene, imine or oxime isomers. Stereoisomeric mixtures include racemic mixtures, diastereomeric mixtures, or E/Z isomeric mixtures. Stereoisomers can be synthesized in pure form (Nogradi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J).

The term “labile” as used herein refers to that which is subject to increased degradation.

The phrase “localized administration”, as used herein, refers to administration of a therapeutic agent in a particular location in the body that may result in a localized pharmacologic effect. Local delivery of a bioactive agent to locations such as organs, cells or tissues can also result in a therapeutically useful, long-lasting presence of a bioactive agent in those local sites or tissues, since the routes by which a bioactive agent is distributed, metabolized, and eliminated from these locations may be different from the routes that define the pharmacokinetic duration of a bioactive agent delivered to the general systemic circulation.

According to some embodiments, delivery is to locations that historically are limited in the volume of administered formulation, that is, only a small amount of formulation volume is capable of being administered. This includes, but is not limited to, local delivery to CNS locations (including, for example, spinal, cerebrospinal or intrathecal delivery or delivery into the brain or to specific sites in and around the brain), and ocular delivery (to sites adjacent to or on the eye, sites within ocular tissue, or intravitreal delivery inside the eye).

The phrase “localized pharmacologic effect”, as used herein, refers to a pharmacologic effect limited to a certain location, i.e. in proximity to a certain location, place, area or site. The phrase “predominantly localized pharmacologic effect”, as used herein, refers to a pharmacologic effect of a drug limited to a certain location by at least 1 to 3 orders of magnitude achieved with a localized administration as compared to a systemic administration.

The term “long-term” release, as used herein, refers to an implant constructed and arranged to deliver therapeutic levels of the active ingredient for at least 7 days, and potentially up to about 30 to about 60 days. Terms such as “long-acting”, “sustained-release” or “controlled release” are used generally to describe a formulation, dosage form, device or other type of technologies used, such as, for example, in the art to achieve the prolonged or extended release or bioavailability of bioactive agent to a subject; it may refer to technologies that provide prolonged or extended release or bioavailability of a bioactive agent to the general systemic circulation or a subject or to local sites of action in a subject including (but not limited to) cells, tissues, organs, joints, regions, and the like. Furthermore, these terms may refer to a technology that is used to prolong or extend the release of a bioactive agent from a formulation or dosage form or they may refer to a technology used to extend or prolong the bioavailability or the pharmacokinetics or the duration of action of a bioactive agent to a subject or they may refer to a technology that is used to extend or prolong the pharmacodynamic effect elicited by a formulation. A “long-acting formulation,” a “sustained release formulation,” or a “controlled release formulation” (and the like) is a pharmaceutical formulation, dosage form, or other technology that is used to provide long-acting release of a bioactive agent to a subject.

Generally, long-acting or sustained release formulations comprise a bioactive agent or agents (including, without limitation nimodipine) that is/are incorporated or associated with a biocompatible polymer in one manner or another. The polymers typically used in the preparation of long-acting formulations include, but are not limited, to biodegradable polymers (such as the polyesters poly(lactide), poly(lactide-co-glycolide), poly(caprolactone), poly(hydroxybutyrates), and the like) and non-degradable polymers (such as ethylenevinyl acetate (EVA), silicone polymers, and the like). The agent may be blended homogeneously throughout the polymer or polymer matrix or the agent may be distributed unevenly (or discontinuously or heterogeneously) throughout the polymer or polymer matrix (as in the case of a bioactive agent-loaded core that is surrounded by a polymer-rich coating or polymer wall forming material as in the case of a microcapsule, nanocapsule, a coated or encapsulated implant, and the like). The dosage form may be in the physical form of particles, film, a fiber, a filament, a cylindrical implant, a asymmetrically-shaped implant, or a fibrous mesh (such as a woven or non-woven material; felt; gauze, sponge, and the like). When in the form of particles, the formulation may be in the form of microparticles, nanoparticles, microparticles, nanospheres, microcapsules or nanocapsules, and particles, in general, and combinations thereof. As such, the long-acting (or sustained-release) formulations of the present invention may include any variety of types or designs that are described, used or practiced in the art.

Long-acting formulations containing bioactive agents can be used to achieve local or site-specific delivery to cells, tissues, organs, bones and the like that are located nearby the site of administration. Further, formulations can be used to achieve systemic delivery of the bioactive agent and/or local delivery of the bioactive agent. Formulations can be delivered by injection (through, for example, needles, syringes, trocars, cannula, and the like) or by implantation. Delivery can be made via any variety of routes of administration commonly used for medical, clinical, surgical purposes including, but not limited to, intravenous, intraarterial, intramuscular, intraperitoneal, subcutaneous, intradermal, infusion and intracatheter delivery (and the like) in addition to delivery to specific locations (such as local delivery) including intrathecal, intracardiac, intraosseous (bone marrow), stereotactic-guided delivery, infusion delivery, CNS delivery, stereo-tactically administered delivery, orthopedic delivery (for example, delivery to joints, into bone, into bone defects and the like), cardiovascular delivery, inter- and intra- and para-ocular (including intravitreal and scleral and retrobulbar and sub-tenons delivery and the like), any delivery to any multitude of other sites, locations, organs, tissues, etc.

The term “manufacture” as used herein refers to all operations of receipt of materials, production, packaging, repackaging, labeling, relabeling, quality control, release, storage and distribution of APIs and related controls.

The term “material” as used herein refers generally to raw materials (e.g., starting materials, reagents, solvents), process aids, intermediates, APIs, packaging and labeling materials.

The term “matrix” as used herein refers to a three dimensional network of fibers that contains voids (or “pores”) where the woven fibers intersect. The structural parameters of the pores, including the pore size, porosity, pore interconnectivity/tortuosity and surface area, affect how substances (e.g., fluid, solutes) move in and out of the matrix.

The term “maximum tolerated dose” as used herein refers to the highest dose of a drug that does not produce unacceptable toxicity.

The term “micronize” and its other grammatical forms as used herein refers to a process that reduces particle size to obtain micrometer- and nanometer-size particles. It may be useful, e.g., to improve the bioavailability of poorly soluble APIs by increasing particle surface area and accelerating dissolution rates; to improve formulation homogeneity and to control particle size. According to some embodiments, the micronization process uses fluid energy, such as a jet mill. A jet mill uses pressurized gas to create high particle velocity and high-energy impact between particles. The process gas is separated from the solid particles after exiting the jet-mill chamber with a cyclone filter. According to some embodiments, the micronization process uses mechanical particle-size reduction, e.g., using a bead mill. Bead milling uses wet mechanical milling to obtain nanoscale particles. In an agitator bead mill, for example, grinding beads and agitating elements are used to reduce the API particle size through impact and shear; product is separated from the grinding media at the outlet. Process parameters include the formulation (e.g., product viscosity, percent solids, additives to prevent reagglomeration), bead density, bead size, bead-filling ratio, stirrer-shaft speed, and flow rate. If containment is needed, the batch-mixing tank can be placed in an isolator, and the mixture can be pumped to the bead mill, which is outside the isolator but is itself a closed system (http://www.pharmtech.com/using-micronization-reduce-api-particle-size).

The term “microparticulate composition”, as used herein, refers to a composition comprising a microparticulate formulation and a pharmaceutically acceptable carrier, where the microparticulate formulation comprises a therapeutic agent and a plurality of microparticles. According to some embodiments, the therapeutic agent is impregnated within the polymer matrix of the microparticles.

The terms “microencapsulated” and “encapsulated” are used herein to refer generally to a bioactive agent that is incorporated into any sort of long-acting formulation or technology regardless of shape or design; therefore, a “microencapsulated” or “encapsulated” bioactive agent may include bioactive agents that are incorporated into a particle or a microparticle and the like or it may include a bioactive agent that is incorporated into a solid implant and so on.

The term “milling” and its other grammatical forms as used herein refers to a process (e.g., a machining process) of grinding, pulverizing, pounding, crushing, pressing, or granulating a solid substance.

The terms “minimum effective concentration”, “minimum effective dose”, or “MEC” are used interchangeably to refer to the minimum concentration of a drug required to produce a desired pharmacological effect in most patients.

The term “modified bioactive agent” as used herein refers, generally, to a bioactive agent that has been modified with another entity through either covalent means or by non-covalent means. The term also is used to include prodrug forms of bioactive agents, where the prodrug form could be a polymeric prodrug or non-polymeric prodrug. Modifications conducted using polymers can be carried out with synthetic polymers (such as polyethylene glycol, PEG; polyvinylpyrrolidone, PVP; polyethylene oxide, PEO; propylene oxide, PPO; copolymers thereof; and the like), biopolymers (such as polysaccharides, proteins, polypeptides, among others) or synthetic or modified biopolymers.

The term “modulate” as used herein means to regulate, alter, adapt, or adjust to a certain measure or proportion.

The term “optical rotation” refers to the change of direction of the plane of polarized light to either the right or the left as it passes through a molecule containing one or more asymmetric carbon atoms or chirality centers. The direction of rotation, if to the right, is indicated by either a plus sign (+) or a d−; if to the left, by a minus (−) or an l−. Molecules having a right-handed configuration (D) usually are dextrorotatory, D(+), but may be levorotatory, L(−). Molecules having left-handed configuration (L) are usually levorotatory, L(−), but may be dextrorotatory, D(+). Compounds with this property are said to be optically active and are termed optical isomers. The amount of rotation of the plane of polarized light varies with the molecule but is the same for any two isomers, though in opposite directions.

The term “parenteral” as used herein refers to a route of administration where the drug or agent enters the body without going through the stomach or “gut”, and thus does not encounter the first pass effect of the liver. Examples include, without limitation, introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), intraventricular injection, intracisternal injection, or infusion techniques. A parenterally administered composition is delivered using a needle.

The term “particles” as used herein refers to an extremely small constituent, e.g., nanoparticles or microparticles) that may contain in whole or in part at least one therapeutic agent as described herein. The term “microparticle” is used herein to refer generally to a variety of substantially structures having sizes from about 10 nm to 2000 microns (2 millimeters) and includes microcapsule, microparticle, nanoparticle, nanocapsule, nanosphere as well as particles, in general, that are less than about 2000 microns (2 millimeters). The particles may contain therapeutic agent(s) in a core surrounded by a coating. Therapeutic agent(s) also may be dispersed throughout the particles. Therapeutic agent(s) also may be adsorbed into the particles. The particles may be of any order release kinetics, including zero order release, first order release, second order release, delayed release, sustained release, immediate release, etc., and any combination thereof. The particles may include, in addition to therapeutic agent(s), any of those materials routinely used in the art of pharmacy and medicine, including, but not limited to, erodible, nonerodible, biodegradable, or nonbiodegradable material or combinations thereof. The particles may be microcapsules that contain the therapeutic agent in a solution or in a semi-solid state. The particles may be of virtually any shape.

The terms “D value” or “mass division diameter” as used herein, refer to the diameter which, when all particles in a sample are arranged in order of ascending mass, divides the sample's mass into specified percentages. The percentage mass below the diameter of interest is the number expressed after the “D”. For example, the D10 diameter is the diameter at which 10% of a sample's mass is comprised of smaller particles, and the D50 is the diameter at which 50% of a sample's mass is comprised of smaller particles. The D50 is also known as the “mass median diameter” as it divides the sample equally by mass. While D-values are based on a division of the mass of a sample by diameter, the actual mass of the particles or the sample does not need to be known. A relative mass is sufficient as D-values are concerned only with a ratio of masses. This allows optical measurement systems to be used without any need for sample weighing.

From the diameter values obtained for each particle a relative mass can be assigned according to the following relationship:

Mass of a sphere=π/6d3ρ

Assuming that p is constant for all particles and cancelling all constants from the equation:

Relative mass=d³

, each particle's diameter is therefore cubed to give its relative mass. These values can be summed to calculate the total relative mass of the sample measured. The values may then be arranged in ascending order and added iteratively until the total reaches 10%, 50% or 90% of the total relative mass of the sample. The corresponding D value for each of these is the diameter of the last particle added to reach the required mass percentage.

The term “pharmaceutical composition” is used herein to refer to a composition that is employed to prevent, reduce in intensity, cure or otherwise treat a target condition or disease.

As used herein the phrase “pharmaceutically acceptable carrier” refers to any substantially non-toxic carrier useable for formulation and administration of the composition of the described invention in which the product of the described invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “pharmacologic effect”, as used herein, refers to a result or consequence of exposure to an active agent.

The term “pilot scale” as used herein refers to the manufacture of either a drug substance or drug product by a procedure fully representative of and simulating that used for full manufacturing scale. In production of microspheres, pilot scale can be, for example, 500 grams. For an API, pilot scale can be, for example 1 kg.

The term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer.

As used herein, the terms “polymorph” or “polymorphic form” are used interchangeably to refer to crystalline forms having the same chemical composition but different spatial arrangements of the molecules, atoms, and/or ions forming the crystal.

The term “process” as used herein refers to a series of operations, actions and controls used to manufacture a drug product.

The term “production” as used herein refers to all operations involved in the preparation of an API from receipt of materials through processing and packaging of the API.

The term “pulsatile release” as used herein refers to any drug-containing formulation in which a burst of the drug is released at one or more predetermined time intervals.

The term “racemate” as used herein refers to an equimolar mixture of two optically active components that neutralize the optical effect of each other and is therefore optically inactive.

The term “reference standard, primary” as used herein refers to a substance that has been shown by an extensive set of analytical tests to be authentic material that should be of high purity. This standard can be, for example, obtained from an officially recognized source; prepared by independent synthesis; obtained from existing production material of high purity; or prepared by further purification of existing production material.

The term “reference standard, secondary,” as used herein refers to a substance of established quality and purity, as shown by comparison to a primary reference standard, used as a reference standard for routine laboratory analysis.

The term “release” and its various grammatical forms, refers to dissolution of an active drug component and diffusion of the dissolved or solubilized species by a combination of the following processes: (1) hydration of a matrix, (2) diffusion of a solution into the matrix; (3) dissolution of the drug; and (4) diffusion of the dissolved drug out of the matrix.

The term “reduce” or “reducing” as used herein refers to a diminution, a decrease, an attenuation, limitation or abatement of the degree, intensity, extent, size, amount, density, number or occurrence of disorder in individuals at risk of developing the disorder.

The term “reprocessed” as used herein refers to introducing an API, including one that does not conform to standards or specifications, back into the process and repeating a crystallization step or other appropriate chemical or physical manipulation steps (e.g., filtration, milling) that are part of the established manufacturing process.

The term “scale-up” as used herein refers to a process of increasing the batch size. For example, without limitation, scale-up can be done in 1:10 ratio for maximum jump scale each time. The term “scale-down” refers to the process of decreasing the batch size.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. A “suspension” is a dispersion (mixture) in which a finely-divided species is combined with another species, with the former being so finely divided and mixed that it doesn't rapidly settle out. In everyday life, the most common suspensions are those of solids in liquid.

The term “solvate” as used herein refers to a complex formed by the attachment of solvent molecules to that of a solute.

The term “solvent” refers to a an inorganic or organic liquid capable of dissolving another substance (termed a “solute”) to form a uniformly dispersed mixture (solution) used as a vehicle for the preparation of solutions or suspensions.

The term “specification” as used herein refers to a list of tests, references to analytical procedures, and appropriate acceptance criteria that are numerical limits, ranges or other criteria for the test described that establishes the set of criteria to which material should conform to be considered acceptable for its intended use. The term “conformance to specification” means that the material, when tested according to the listed analytical procedures, will meet the listed acceptance criteria.

The term “subarachnoid cavity” or “subarachnoid space” refers to the space between the outer cellular layer of the arachnoid and the pia mater occupied by tissue consisting of trabeculae of delicate connective tissue and intercommunicating channels in which the cerebrospinal fluid is contained. This cavity is small on the surface of the hemispheres of the brain; on the summit of each gyrus the pia mater and the arachnoid are in close contact; but triangular spaces are left in the sulci between the gyri, in which the subarachnoid trabecular tissue is found, because the pia mater dips into the sulci, whereas the arachnoid bridges across them from gyrus to gyrus. At certain parts of the base of the brain, the arachnoid is separated from the pia mater by wide intervals, which communicate freely with each other and are named subarachnoid cisternae; the subarachnoid tissue in these cisternae is less abundant.

The subarachnoid cisternae (cisternae subarachnoidales) include the cisterna cerebellomedularis, the cisterna pontis, the cisterna interpeduncularis, cisterna chiasmatis, cisterna fossae cerebri lateralis and cisterna venae magnae cerebri.

The cisterna cerebellomedullaris (cisterna magna) is triangular on sagittal section, and results from the arachnoid bridging over the space between the medulla oblongata and the under surfaces of the hemispheres of the cerebellum; it is continuous with the subarachnoid cavity of the spinal cord at the level of the foramen magnum.

The cisterna pontis is a considerable space on the ventral aspect of the pons. It contains the basilar artery, and is continuous behind the pons with the subarachnoid cavity of the spinal cord, and with the cisterna cerebellomedullaris; in front of the pons, it is continuous with the cisterna interpeduncularis.

The cisterna interpeduncularis (cisterna basalis) is a wide cavity where the arachnoid extends across between the two temporal lobes. It encloses the cerebral peduncles and the structures contained in the interpeduncular fossa, and contains the arterial circle of Willis. In front, the cisterna interpeduncularis extends forward across the optic chiasma, forming the cisterna chiasmatis, and on to the upper surface of the corpus callosum. The arachnoid stretches across from one cerebral hemisphere to the other immediately beneath the free border of the falx cerebri, and thus leaves a space in which the anterior cerebral arteries are contained. The cisterna fossae cerebri lateralis is formed in front of either temporal lobe by the arachnoid bridging across the lateral fissure. This cavity contains the middle cerebral artery. The cisterna venae magnae cerebri occupies the interval between the splenium of the corpus callosum and the superior surface of the cerebellum; it extends between the layers of the tela chorioidea of the third ventricle and contains the great cerebral vein.

The subarachnoid cavity communicates with the general ventricular cavity of the brain by three openings; one, the foramen of Majendie, is in the middle line at the inferior part of the roof of the fourth ventricle; the other two (the foramina of Luschka) are at the extremities of the lateral recesses of that ventricle, behind the upper roots of the glossopharyngeal nerves.

The term “subarachnoid hemorrhage” or “SAH” is used herein to refer to a condition in which blood collects beneath the arachnoid mater. This area, called the subarachnoid space, normally contains cerebrospinal fluid. The accumulation of blood in the subarachnoid space may lead to stroke, seizures, and other complications. Additionally, SAH may cause permanent brain damage and a number of harmful biochemical events in the brain. Causes of SAH include bleeding from a cerebral aneurysm, vascular anomaly, trauma and extension into the subarachnoid space from a primary intracerebral hemorrhage. Symptoms of SAH include, for example, sudden and severe headache, nausea and/or vomiting, symptoms of meningeal irritation (e.g., neck stiffness, low back pain, bilateral leg pain), photophobia and visual changes, and/or loss of consciousness. SAH is often secondary to a head injury or a blood vessel defect known as an aneurysm. In some instances, SAH can induce cerebral vasospasm that may in turn lead to an ischemic stroke. A common manifestation of a SAH is the presence of blood in the CSF. Subjects having a SAH may be identified by a number of symptoms. For example, a subject having an SAH will present with blood in the subarachnoid space. Subjects having an SAH can also be identified by an intracranial pressure that approximates mean arterial pressure at least during the actual hemorrhage from a ruptured aneurysm, by a fall in cerebral perfusion pressure, or by the sudden severe headache, sudden transient loss of consciousness (sometimes preceded by a painful headache), sudden loss of consciousness or sometimes sudden collapse and death. In about half of cases, subjects present with a severe headache which may be associated with physical exertion. Other symptoms associated with subarachnoid hemorrhage include nausea, vomiting, memory loss, hemiparesis and aphasia. Subjects having a SAH also may be identified by the presence of creatine kinase-BB isoenzyme activity in their CSF. This enzyme is enriched in the brain but normally is not present in the CSF. Thus, its presence in the CSF is indicative of “leak” from the brain into the subarachnoid space. Assay of creatine-kinase BB isoenzyme activity in the CSF is described by Coplin et al. (Coplin et al 1999 Arch Neurol 56, 1348-1352) Additionally, a spinal tap or lumbar puncture may be used to demonstrate whether blood is present in the CSF, a strong indication of an SAH. A cranial CT scan or an MRI also may be used to identify blood in the subarachnoid region. Angiography also may be used to determine not only whether a hemorrhage has occurred, but also the location of the hemorrhage. Subarachnoid hemorrhage commonly results from rupture of an intracranial saccular aneurysm or from malformation of the arteriovenous system in the brain. Accordingly, a subject at risk of having an SAH includes a subject having a saccular aneurysm as well as a subject having a malformation of the arteriovenous system. Common sites of saccular aneurysms are the anterior communicating artery region, the origin of the posterior communicating artery from the internal carotid artery, the middle cerebral artery, the top of the basilar artery and the junction of the basilar artery with the superior cerebellar or the anterior inferior cerebellar artery. Subjects having SAH may be identified by an eye examination, whereby hemorrhage into the vitreous humor or slowed eye movement may indicate brain damage. A subject with a saccular aneurysm may be identified through routine medical imaging techniques, such as CT and MRI. A saccular or cerebral aneurysm forms a mushroom-like or berry-like shape (sometimes referred to as “a dome with a neck” shape).

The terms “subject” or “individual” or “patient” are used interchangeably to refer to a member of an animal species of mammalian origin, including humans.

The phrase “a subject having microthromboemboli” as used herein refers to a subject who presents with diagnostic markers associated with microthromboemboli. Diagnostic markers include, but are not limited to, the presence of blood in the CSF and/or a recent history of a SAH and/or development of neurological deterioration one to 14 days after SAH when the neurological deterioration is not due to another cause that can be diagnosed, including but not limited to seizures, hydrocephalus, increased intracranial pressure, infection, intracranial hemorrhage or other systemic factors. Another diagnostic marker may be embolic signals detected on transcranial Doppler ultrasound of large conducting cerebral arteries. Microthromboemboli-associated symptoms include, but are not limited to, paralysis on one side of the body, inability to vocalize the words or to understand spoken or written words, and inability to perform tasks requiring spatial analysis. Such symptoms may develop over a few days, or they may fluctuate in their appearance, or they may present abruptly.

The phrase “a subject having cortical spreading ischemia” as used herein means refers to a subject who presents with diagnostic markers associated with cortical spreading ischemia. Diagnostic markers include, but are not limited to, the presence of blood in the CSF and/or a recent history of a SAH and/or development of neurological deterioration one to 14 days after SAH when the neurological deterioration is not due to another cause that can be diagnosed, including but not limited to seizures, hydrocephalus, increased intracranial pressure, infection, intracranial hemorrhage or other systemic factors. Another diagnostic marker may be detection of propagating waves of depolarization with vasoconstriction detected by electrocorticography. Cortical spreading ischemia-associated symptoms include, but are not limited to, paralysis on one side of the body, inability to vocalize the words or to understand spoken or written words, and inability to perform tasks requiring spatial analysis. Such symptoms may develop over a few days, or they may fluctuate in their appearance, or they may present abruptly.

A subject at risk of DCI due to microthromboemboli, cortical spreading ischemia, or angiographic vasospasm or a combination thereof is one who has one or more predisposing factors to the development of these conditions. A predisposing factor includes, but is not limited to, existence of a SAH. A subject who has experienced a recent SAH is at significantly higher risk of developing angiographic vasospasm and DCI than a subject who has not had a recent SAH. MR angiography, CT angiography and catheter angiography can be used to diagnose at least one of DCI, microthromboemboli, cortical spreading ischemia or angiographic vasospasm. Angiography is a technique in which a contrast agent is introduced into the blood stream in order to view blood flow and/or arteries. A contrast agent is required because blood flow and/or arteries sometimes are only weakly apparent in a regular MR scan, CT scan or radiographic film for catheter angiography. Appropriate contrast agents will vary depending upon the imaging technique used. For example, gadolinium is commonly used as a contrast agent used in MR scans. Other MR appropriate contrast agents are known in the art.

As used herein, the term “substantially pure” with reference to a particular polymorphic form means that the polymorphic form includes less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% by weight of any other physical forms of the compound.

The term “same” as used herein refers to agreeing in kind, amount; unchanged in character or condition.

The term “similar” as used herein refers to having a general likeness.

By “sufficient amount” and “sufficient time” means an amount and time needed to achieve the desired result or results, e.g., dissolve a portion of the polymer.

The term “surfactant” or “surface-active agent” as used herein refers to an agent, usually an organic chemical compound that is at least partially amphiphilic, i.e., typically containing a hydrophobic tail group and hydrophilic polar head group

The term “susceptible” as used herein refers to being at risk for.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. Alternatively, delayed absorption of a parenterally administered drug form is accomplished by dissolving or suspending the drug in an oil vehicle. Nonlimiting examples of sustained release biodegradable polymers include polyesters, polyester polyethylene glycol copolymers, polyamino-derived biopolymers, polyanhydrides, hydrogels, polyorthoesters, polyphosphazenes, SAIB, photopolymerizable biopolymers, protein polymers, collagen, polysaccharides, chitosans, and alginates.

The term “symptom” as used herein refers to a phenomenon that arises from and accompanies a particular disease or disorder and serves as an indication of it.

The term “technical grade” as used herein, with respect to excipients refers to excipients that may differ in specifications and/or functionality, impurities, and impurity profiles.

The term “therapeutic agent” as used herein refers to a drug, molecule, composition or other substance that provides a therapeutic effect. The terms “therapeutic agent” and “active agent” are used interchangeably.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “therapeutically effective amount”, “effective amount”, or an “amount effective” is an amount that is sufficient to provide the intended benefit of treatment. Combined with the teachings provided herein, by weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. A therapeutically effective amount of the active agents that can be employed ranges from a unit dose of about 40 mg to about 1000 mg, with a maximum tolerated dose of 800 mg. The therapeutically effective amount for any particular application may vary depending on such factors as the disease or condition being treated, the particular calcium channel inhibitor, calcium channel antagonist, transient receptor potential protein antagonist, or endothelin antagonist being administered, the size of the subject, or the severity of the disease or condition. One of ordinary skill in the art may determine empirically the effective amount of a particular inhibitor and/or other therapeutic agent without necessitating undue experimentation. It is preferred generally that a maximum dose be used, that is, the highest safe dose according to some medical judgment. “Dose” and “dosage” are used interchangeably herein.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “cerebral ventricle” as used herein refers to chambers in the brain that contain cerebrospinal fluid, include two lateral ventricles, one third ventricle, and one fourth ventricle. The lateral ventricles are in the cerebral hemispheres. They drain via the foramen of Monroe into the third ventricle, which is located between the two diencephalic structures of the brain. The third ventricle leads, by way of the aqueduct of Sylvius, to the fourth ventricle. The fourth ventricle is in the posterior fossa between the brainstem and the cerebellum. The cerebrospinal fluid drains out of the fourth ventricle through the foramenae of Luschka and Magendie to the basal cisterns. The cerebrospinal fluid then percolates through subarachnoid cisterns and drains out via arachnoid villi into the venous system.

The term “validation” as used herein refers to establishing through documented evidence a high degree of assurance that a specific process will consistently produce a product that meets its predetermined specifications and quality attributes. A validated manufacturing process is one that has been proven to do what it purports or is represented to do. The proof of validation is obtained through collection and evaluation of data, e.g., beginning from the process development phase and continuing through into the production phase. Validation includes process qualification (meaning the qualification of materials, equipment, systems, buildings and personnel), and the control of entire processes for repeated batches or runs.

The term “viscosity”, as used herein refers to the property of a fluid that resists the force tending to cause the fluid to flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm2, and 1 cP=0.01 P. Kinematic viscosity is the ratio of absolute or dynamic viscosity to density. Kinematic viscosity is usually denoted in Stoke (St) or Centistokes (cSt), wherein 1 St=10-4 m2/s, and 1 cSt=0.01 St.

As used herein, a “wt. %” or “weight percent” or “percent by weight” of a component, unless specifically stated to the contrary, refers to the ratio of the weight of the component to the total weight of the composition in which the component is included, expressed as a percentage.

The term “expected yield” as used herein refers to the quantity of material or the percentage of theoretical yield anticipated at any appropriate phase of production, based on previous laboratory, pilot scale, or manufacturing data. The term “theoretical yield” as used herein refer to the quantity that would be produced at any appropriate phase of production based on the quantity of material to be used in the absence of any loss or error in actual production.

Particulate Formulation

According to some embodiments, a biocompatible polymeric or non-polymeric system is utilized to prepare a particulate component of a particulate formulation containing particles and a therapeutic agent, which are formulated into a pharmaceutical composition for site specific delivery. Following final processing methods, the particulate composition can be delivered locally, e.g., intracisternally, intraventricularly, or intrathecally into the cerebrospinal fluid from which the therapeutic agent subsequently is released by drug release mechanisms.

### API

According to some embodiments the API starting material is the dihydropyridine L-type voltage dependent calcium channel inhibitor nimodipine.

According to some embodiments, the API starting material is a substantially pure crystalline form I of nimodipine. According to some such embodiments, the substantially pure crystalline form I of nimodipine contains less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, or less than 5%) of any other form of nimodipine (e.g., conglomerate form II of nimodipine, an amorphous form of nimodipine or a combination thereof).

According to some embodiments, the API starting material is a substantially pure polymorphic form II of nimodipine. According to some embodiments, the substantially pure polymorphic Form II of nimodipine is at least 85%, at least 90%, at least 95%, at least 96%, at least 97%, at least 98%, at least 99% nimodipine form II.

According to some embodiments, the particle size of the API starting material can be controlled by miling, micronizing, or both. According to some embodiments, the API specification for particle size includes D10>2 μm, D50=7 μm, and D90<10 μm.

### Polymer

Exemplary criteria for selection of a polymer(s) for use in the described microparticulate formulations include, without limitation, the type of polymer, the selection of a co-polymer, the type of co-monomers used in the co-polymer, the ratio of the types of monomers used in the co-polymer, the molecular weight of the polymer, the size of the microparticle, and any other criteria used by one of skill in the art to control the release profile of a microparticle.

Both non-biodegradable and biodegradable polymeric materials may be used in the manufacture of particles for delivering a therapeutic agent of the described invention. Such polymers may be natural or synthetic polymers. The polymer is selected based on the period of time over which release is desired.

Exemplary bioadhesive polymers include bioerodible hydrogels as described by Sawhney et al in Macromolecules (1993) 26, 581-587. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Exemplary biocompatible non-degradable polymers include, without limitation, polyacrylates; a polymer of ethylene-vinyl acetate, EVA; cellulose acetate; an acyl-substituted cellulose acetate; a non-degradable polyurethane; a polystyrene; a polyvinyl chloride; a polyvinyl fluoride; a poly(vinyl imidazole); a silicone-based polymer (for example, Silastic® and the like), a chlorosulphonate polyolefin; a polyethylene oxide; or a blend or copolymer thereof.

Exemplary biocompatible biodegradable polymers include, without limitation, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a polyanhydride; a poly(phosphazene); a polyhydroxyalkanoate; a poly(hydroxybutyrate); a poly(hydroxybutyrate) synthetically derived; a poly(hydroxybutyrate) biologically derived; a polyester synthetically derived; a polyester biologically derived; a poly(lactide-co-caprolactone); a poly(lactide-co-glycolide-co-caprolactone); a polycarbonate; a tyrosine polycarbonate; a polyamide (including synthetic and natural polyamides, polypeptides, poly(amino acids) and the like); a polyesteramide; a polyester; a poly(dioxanone); a poly(alkylene alkylate); a polyether (such as polyethylene glycol, PEG, and polyethylene oxide, PEO); polyvinyl pyrrolidone or PVP; a polyurethane; a polyetherester; a polyacetal; a polycyanoacrylate; a poly(oxyethylene)/poly(oxypropylene) copolymer; a polyacetal, a polyketal; a polyphosphate; a (phosphorous-containing) polymer; a polyphosphoester; a polyhydroxyvalerate; a polyalkylene oxalate; a polyalkylene succinate; and a poly(maleic acid).

Exemplary biopolymers or modified biopolymers include chitin, chitosan, modified chitosan, among other biocompatible polysaccharides; or biocompatible copolymers (including block copolymers or random copolymers) herein; or combinations or mixtures or admixtures of any polymers herein.

Exemplary copolymers include block copolymers containing blocks of hydrophilic or water-soluble polymers (such as polyethylene glycol, PEG, or polyvinyl pyrrolidone, PVP) with blocks of other biocompatible or biodegradable polymers (for example, poly(lactide) or poly(lactide-co-glycolide or polycaprolcatone or combinations thereof).

Exemplary long-acting formulations prepared from copolymers include those comprised of the monomers of lactide (including L-lactide, D-lactide, and combinations thereof) or hydroxybutyrates or caprolactone or combinations thereof; long-acting formulations prepared from copolymers that are comprised of the monomers of DL-lactide, glycolide, hydroxybutyrate, and caprolactone and long-acting formulations prepared from copolymers comprised of the monomers of DL-lactide or glycolide or caprolactone or hydroxybutyrates or combinations thereof. Additionally, long-acting formulations may be prepared from admixtures containing the aforementioned copolymers (comprised of DL-lactide or glycolide or caprolactone or hydroxybutyrates or combinations therein) along with other biodegradable polymers including poly(DL-lactide-co-glycolide) or poly(DL-lactide) or PHA's, among others. Long-acting formulations also may be prepared from block copolymers comprising blocks of either hydrophobic or hydrophilic biocompatible polymers or biopolymers or biodegradable polymers such as polyethers (including polyethylene glycol, PEG; polyethylene oxide, PEO; polypropylene oxide, PPO and block copolymers comprised of combinations thereof) or polyvinyl pyrrolidone (PVP), polysaccharides, conjugated polysaccharides, modified polysaccharides, such as fatty acid conjugated polysaccharides, polylactides, polyesters, among others.

Injectable depot forms can be made by forming microencapsulated matrices of the drug in biodegradable polymers such as polylactide-polyglycolide. Depending upon the ratio of drug to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations are also prepared by entrapping the drug in liposomes or microemulsions which are compatible with body tissues.

For example, polyglycolide (PGA) is a linear aliphatic polyester developed for use in sutures. Studies have reported PGA copolymers formed with trimethylene carbonate, polylactic acid (PLA), and polycaprolactone. Some of these copolymers may be formulated as microparticles for sustained drug release.

For example, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available. Lactide/glycolide polymers can be conveniently made by melt polymerization through ring opening of lactide and glycolide monomers.

Polyester-polyethylene glycol compounds can be synthesized; these are soft and may be used for drug delivery.

Poly (amino)-derived biopolymers may include, but are not limited to, those containing lactic acid and lysine as the aliphatic diamine (see, for example, U.S. Pat. No. 5,399,665), and tyrosine-derived polycarbonates and polyacrylates. Modifications of polycarbonates may alter the length of the alkyl chain of the ester (ethyl to octyl), while modifications of polyarylates may further include altering the length of the alkyl chain of the diacid (for example, succinic to sebasic), which allows for a large permutation of polymers and great flexibility in polymer properties.

Polyanhydrides are prepared by the dehydration of two diacid molecules by melt polymerization (see, for example, U.S. Pat. No. 4,757,128). These polymers degrade by surface erosion (as compared to polyesters that degrade by bulk erosion). The release of the drug can be controlled by the hydrophilicity of the monomers chosen.

Photopolymerizable biopolymers include, but are not limited to, lactic acid/polyethylene glycol/acrylate copolymers.

According to some embodiments, the polymer forms a matrix (hereinafter the polymer matrix) with the therapeutic agent so as to obtain a desired release pattern of the active ingredient. According to some embodiments, the therapeutic agent is impregnated in or the polymer matrix. According to some embodiments, the polymer matrix encapsulates the therapeutic agent. According to some embodiments, the polymer matrix is homogeneous and contains a single polymer. According to some embodiments, the polymer matrix contains a first polymer and a second polymer. According to some embodiments, more than two polymers can be present in a blend, for example, 3, 4, 5, or more polymers can be present. According to some embodiments, the polymer matrix comprises cross-linked or intertwined polymer chains.

According to some embodiments, the matrix comprises a photopolymerizable biopolymer. Exemplary photopolymerizable biopolymers include, without limitation, lactic acid/polyethylene glycol/acrylate copolymers.

According to some embodiments, the matrix comprises a hydrogel. The term “hydrogel” refers to a substance resulting in a solid, semisolid, pseudoplastic or plastic structure containing a necessary aqueous component to produce a gelatinous or jelly-like mass. Hydrogels generally comprise a variety of polymers, including hydrophilic polymers, acrylic acid, acrylamide and 2-hydroxyethylmethacrylate (HEMA). Many hydrogels, polymers, hydrocarbon compositions and fatty acid derivatives having similar physical/chemical properties with respect to viscosity/rigidity may function as a semisolid delivery system. According to some embodiments, the hydrogel incorporates and retains significant amounts of water, which eventually will reach an equilibrium content in the presence of an aqueous environment.

According to some embodiments, the matrix comprises a naturally-occurring biopolymer. Exemplary naturally-occurring biopolymers include, but are not limited to, protein polymers, collagen, polysaccharides, and photopolymerizable compounds.

According to some embodiments, the matrix comprises a protein polymer. Exemplary protein polymers synthesized from self-assembling protein polymers include, for example, silk fibroin, elastin, collagen, and combinations thereof.

According to some embodiments, the matrix comprises a naturally-occurring polysaccharide. Exemplary naturally-occurring polysaccharides include, but are not limited to, chitin and its derivatives, hyaluronic acid, dextran and cellulosics (which generally are not biodegradable without modification), and sucrose acetate isobutyrate (SAIB). Hyaluronic acid (HA), which is composed of alternating glucuronidic and glucosaminidic bonds and is found in mammalian vitreous humor, extracellular matrix of the brain, synovial fluid, umbilical cords and rooster combs from which it is isolated and purified, also can be produced by fermentation processes.

According to some embodiments, the matrix comprises a chitin matrix. Chitin is composed predominantly of 2-acetamido-2-deoxy-D-glucose groups and is found in yeast, fungi and marine invertebrates (shrimp, crustaceous) where it is a principal component of the exoskeleton. Chitin is not water soluble and the deacetylated chitin, chitosan, only is soluble in acidic solutions (such as, for example, acetic acid). Studies have reported chitin derivatives that are water soluble, very high molecular weight (greater than 2 million Daltons), viscoelastic, non-toxic, biocompatible and capable of crosslinking with peroxides, gluteraldehyde, glyoxal and other aldehydes and carbodiamides, to form gels. Depending on the desired degradation profile of the controlled release system, a wide variety of properties differ among the polymers, including without limitation, chemical composition, viscosity (e.g., inherent viscosity), molecular weight, thermal properties, such as glass transition temperature (T_{g}), the chemical composition of a non-repeating unit therein, such as an end group, degradation rate, hydrophilicity, porosity, density, or a combination thereof. According to some embodiments, the first polymer and the second polymer have different degradation rates in an aqueous medium. According to some embodiments, a degradation profile of a controlled release system and a combination of polymers is selected so that, when combined, the polymers achieve the selected degradation profile.

According to some embodiments, a first polymer and a second polymer of the polymer matrix comprise one or more different non-repeating units, such as, for example, an end group, or a non-repeating unit in the backbone of the polymer. According to some embodiments, the first polymer and the second polymer of the polymer matrix comprise one or more different end groups. For example, the first polymer can have a more polar end group than one or more end group(s) of the second polymer. According to some such embodiments, the first polymer will be more hydrophilic and thus lead to faster water uptake, relative to a controlled release system comprising the second polymer (with the less polar end group) alone. According to some such embodiments, the first polymer comprises one or more carboxylic acid end groups, and the second polymer comprises have one or more ester end groups. According to some such embodiments, a single polymer can have one or more ester or carboxylic end groups depending on the desire for faster water uptake or a more controlled release system.

According to some embodiments, the first polymer and the second polymer of the polymer matrix are of different molecular weights. Without being limited by theory, it is generally understood that the greater the molecular weight of the polymer, the more viscous the polymer is. As viscosity increases the selection for a more purified polymeric form increases. For example, according to some embodiments, the first polymer has a molecular weight that is at least about 3000 Daltons greater than the molecular weight of the second polymer. The molecular weight can have any suitable value, which can, in various aspects, depend on the desired properties of the controlled release system. If, for example, a controlled release system having high mechanical strength is desired, at least one of the polymers can have a high molecular weight. If it is also desired that the controlled release system have short term release capability (e.g., less than about 2 weeks), then a lower molecular weight polymer can be combined with the high molecular weight polymer. The high molecular weight polymer typically will provide good structural integrity for the controlled release system, while the lower molecular weight polymer can provide short term release capability.

According to some embodiments, the first and second polymer of the polymer matrix can be present in the polymer mixture in any desired ratio, e.g., the weight ratio of the first polymer to the second polymer or the mole ratio of the first polymer to the second polymer. According to some embodiments, the weight ratio of the first polymer to the second polymer is from about 90:10 to about 40:60, including, without limitation, weight ratios of about 85:15, 80:20, 70:30, 75:25, 65:35, and 50:50, among others.

When the biodegradable polymer is poly(lactide-co-glycolide), poly(lactide), or poly(glycolide), the amount of lactide and glycolide in the polymer can vary. For example, according to some embodiments, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. According to some embodiments, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

It is understood that any combination of the aforementioned biodegradable polymers can be used, including, but not limited to, copolymers, mixtures, or blends thereof.

### Particulate Formulation

According to some embodiments, the particulate composition comprises a particulate formulation containing a plurality of particles. According to some embodiments, the particulate formulation comprises a plurality of milliparticles comprising a therapeutic amount of a therapeutic agent, wherein the therapeutic agent is dispersed throughout each milliparticle, adsorbed onto the milliparticles, or is in a core surrounded by a coating. According to some embodiments, the particulate formulation comprises a plurality of microparticles comprising a therapeutic amount of a first therapeutic agent, wherein the first therapeutic agent is dispersed throughout each microparticle, adsorbed onto the microparticles, or in a core surrounded by a coating. According to some embodiments, the particulate formulation comprises a plurality of nanoparticles comprising a therapeutic amount of a first therapeutic agent, wherein the first therapeutic agent is dispersed throughout each nanoparticle, adsorbed onto the nanoparticles, or in a core surrounded by a coating. According to some embodiments, the particulate formulation comprises a plurality of picoparticles comprising a therapeutic amount of a first therapeutic agent, wherein the first therapeutic agent is dispersed throughout each picoparticle, adsorbed onto the picoparticles, or in a core surrounded by a coating. According to some embodiments, the particulate formulation comprises a plurality of femtoparticles comprising a therapeutic amount of a first therapeutic agent, wherein the first therapeutic agent is dispersed throughout each femtoparticle, adsorbed onto the femtoparticles, or in a core surrounded by a coating.

According to some embodiments, the particles of the particulate formulation are of a uniform distribution of particle size. According to some embodiments, the uniform distribution of particle size is achieved by a non-emulsion based homogenization process. According to some embodiments, the uniform distribution of particle size is achieved by an emulsion based process to form a uniform emulsion.

According to some embodiments, the microparticle formulation comprises a uniform distribution of microparticles from about 10 μm to about 100 μm in particle size. According to some embodiments, at least 5%, 10%, 15%, 30%, 25%, 30%, 35%, 40%, 45%, 50%, 55%, 60%, 65%, 70%, 75%, 80%, 85%, 90%, or 95% of the microparticles are of a size greater than 10 μm. According to some embodiments, at least 5%, 10%, 15%, 30%, 25%, 30%, 35%, 40%, 45%, 50%, 55%, 60%, 65%, 70%, 75%, 80%, 85%, 90% or 95% of the microparticles are of a size greater than 25 μm. According to some embodiments, at least 5%, 10%, 15%, 30%, 25%, 30%, 35%, 40%, 45%, 50%, 55%, 60%, 65%, 70%, 75%, 80%, 85%, 90% or 95% of the microparticles are of a size greater than 50 μm. According to some embodiments, at least 5%, 10%, 15%, 30%, 25%, 30%, 35%, 40%, 45%, 50%, 55%, 60%, 65%, 70%, 75%, 80%, 85%, 90%, or 95% of the microparticles are of a size greater than 75 μm. According to some embodiments, at least 5%, 10%, 15%, 30%, 25%, 30%, 35%, 40%, 45%, 50%, 55%, 60%, 65%, 70%, 75%, 80%, 85%, 90% or 95% of the microparticles are of a size less than 90 μm. According to some embodiments, at least 5%, 10%, 15%, 30%, 25%, 30%, 35%, 40%, 45%, 50%, 55%, 60%, 65%, 70%, 75%, 80%, 85%, 90% or 95% of the microparticles are of a size less than 75 μm. According to some embodiments, at least 5%, 10%, 15%, 20%, 25%, 30%, 35%, 40%, 45%, 50%, 55%, 60%, 65%, 70%, 75%, 80%, 85%, 90% or 95% of the microparticles are of a size less than 50 μm.

According to some embodiments, the API specification for the microparticles comprising substantially pure Nimodipine form II includes D10>20 μm, D50 of 70-80 μm, and D90<200 μm.

According to another embodiment, the average particle size is at least about 10 μm. According to another embodiment, the average particle size is at least about 15 μm. According to another embodiment, the average particle size is at least about 20 μm. According to another embodiment, the average particle size is at least about 25 μm. According to another embodiment, the average particle size is at least about 30 μm. According to another embodiment, the average particle size is at least about 35 μm. According to another embodiment, the average particle size is at least about 40 μm. According to another embodiment, the average particle size is at least about 45 μm. According to another embodiment, the average particle size is at least about 50 μm. According to another embodiment, the average particle size is at least about 55 μm. According to another embodiment, the average particle size is at least about 60 μm. According to another embodiment, the average particle size is at least about 65 μm. According to another embodiment, the average particle size is at least about 70 μm. According to another embodiment, the average particle size is at least about 75 μm. According to another embodiment, the average particle size is at least about 80 μm. According to another embodiment, the average particle size is at least about 85 μm. According to another embodiment, the average particle size is at least about 90 μm. According to another embodiment, the average particle size is at least about 95 μm. According to another embodiment, the average particle size is at least about 100 μm. According to another embodiment, the average particle size is at least about 110 μm. According to another embodiment, the average particle size is at least about 115 μm. According to another embodiment, the average particle size is at least about 120 μm. According to another embodiment, the average particle size is at least about 125 μm. According to another embodiment, the average particle size is at least about 130 μm. According to another embodiment, the average particle size is at least about 135 μm. According to another embodiment, the average particle size is at least about 140 μm. According to another embodiment, the average particle size is at least about 145 μm. According to another embodiment, the average particle size is at least about 150 μm. According to another embodiment, the average particle size is at least about 155 μm. According to another embodiment, the average particle size is at least about 160 μm. According to another embodiment, the average particle size is at least about 165 μm. According to another embodiment, the average particle size is at least about 170 μm. According to another embodiment, the average particle size is at least about 175 μm. According to another embodiment, the average particle size is at least about 180 μm. According to another embodiment, the average particle size is at least about 185 μm. According to another embodiment, the average particle size is at least about 190 μm. According to another embodiment, the average particle size is at least about 195 μm. According to another embodiment, the average particle size is at least about 200 μm.

According to some embodiments, the therapeutic agent is disposed on or in the particles. According to some embodiments, the therapeutic agent is dispersed throughout the particles. According to some embodiments, the particles are impregnated with the therapeutic agent. According to some embodiments, the therapeutic agent is adsorbed onto a surface of the particles. According to some embodiments, the therapeutic agent is contained within a core of the particles surrounded by a coating. According to some embodiments, the particles comprise a matrix. According to some embodiments, the matrix comprises the therapeutic agent. According to some embodiments, the matrix is impregnated with the therapeutic agent.

According to some embodiments, the particles can be of any order release kinetics, including zero order release, first order release, second order release, delayed release, sustained release, immediate release, and a combination thereof. In addition to therapeutic agent(s), the particles can include any of those materials routinely used in the art of pharmacy and medicine, including, but not limited to, erodible, nonerodible, biodegradable, or nonbiodegradable material or combinations thereof.

According to some embodiments, the therapeutic agent formulated into the pharmaceutical composition for site-specific delivery, comprises substantially pure polymorphic Form II of nimodipine. According to some embodiments, the substantially pure polymorphic Form II of nimodipine contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94% at least 95%, at least 96%, at least 97%, at least 98%, at least 99% form II.

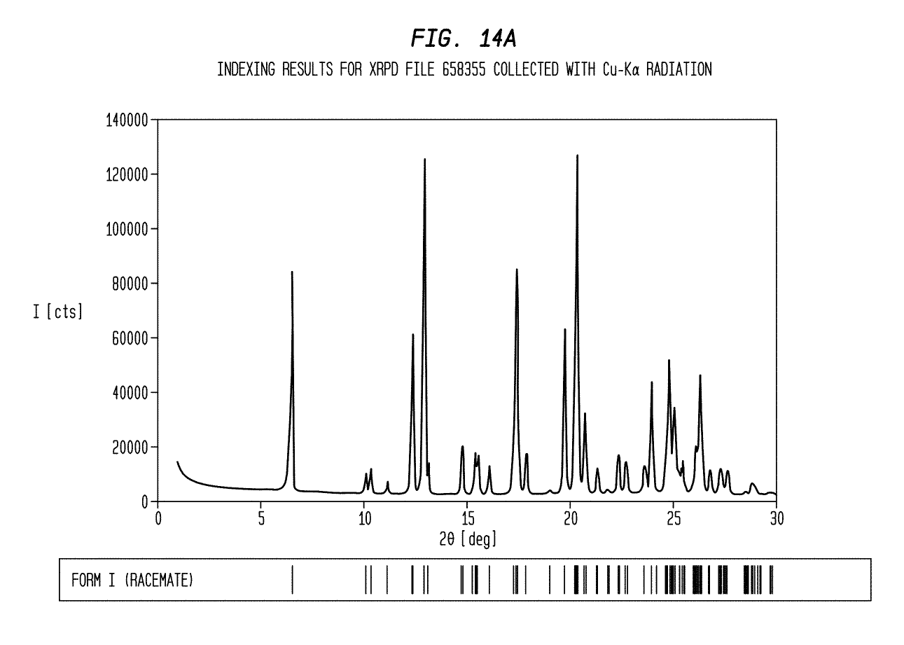

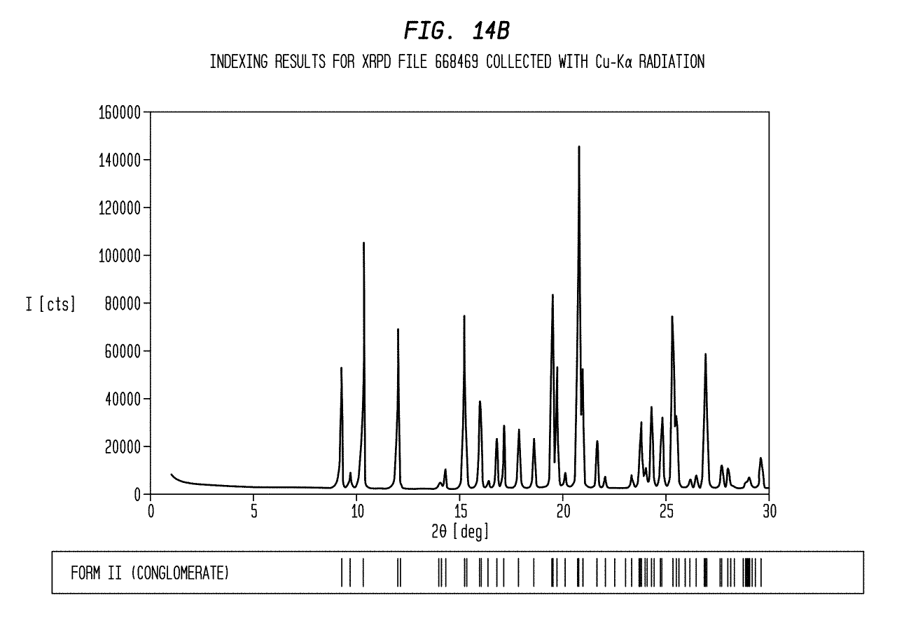

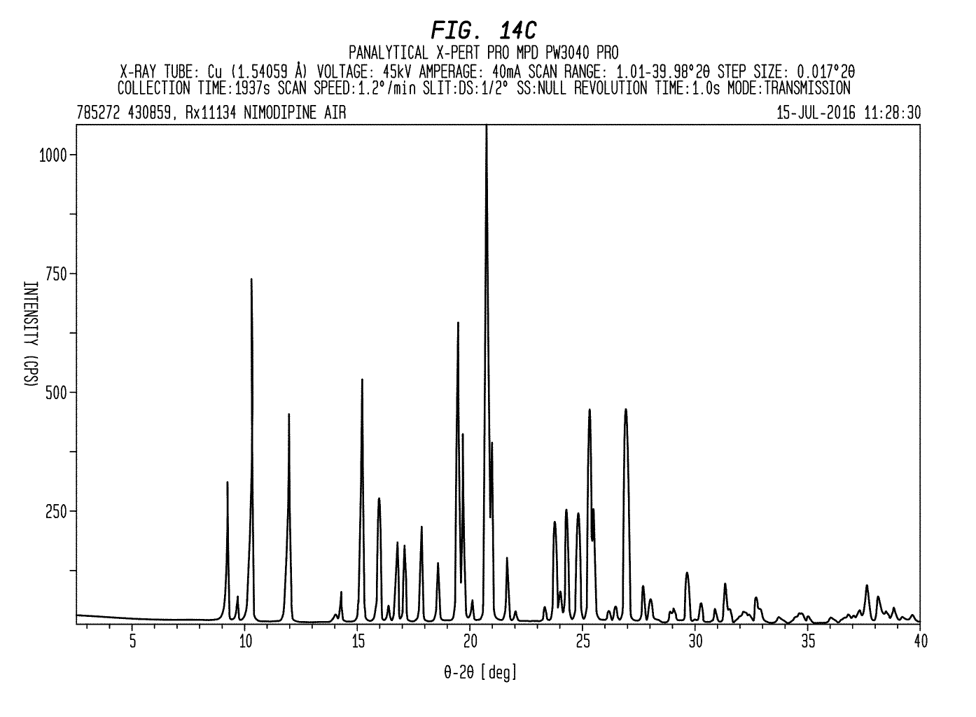

According to some embodiments, the substantially pure polymorphic Form II of nimodipine is characterized by an X-ray diffraction pattern as shown in FIG. 14B. According to some embodiments, the substantially pure polymorphic Form II of nimodipine is characterized by a melting temperature of +116±1° C. as determined by differential scanning calorimetry. According to some embodiments, the substantially pure polymorphic Form II of nimodipine is characterized by both an X-ray diffraction pattern as shown in FIG. 14B and by a melting temperature of +116±1° C. as determined by differential scanning calorimetry.

According to some embodiments, the particles are loaded with an average of at least 5% by weight of the therapeutic agent. According to some embodiments, the particles are loaded with an average of at least 10% by weight of the therapeutic agent. According to some embodiments, the particles are loaded with an average of at least 15% by weight of the therapeutic agent. According to some embodiments, the particles are loaded with an average of at least 20% by weight of the therapeutic agent. According to some embodiments, the particles are loaded with an average of at least 25% by weight of the therapeutic agent. According to some embodiments, the particles are loaded with an average of at least 30% by weight of the therapeutic agent. According to some embodiments, the particles are loaded with an average of at least 35% by weight of the therapeutic agent. According to some embodiments, the particles are loaded with an average of at least 40% by weight of the therapeutic agent. According to some embodiments, the particles are loaded with an average of at least 45% by weight of the therapeutic agent. According to some embodiments, the particles are loaded with an average of at least 50% by weight of the therapeutic agent. According to some embodiments, the particles are loaded with an average of at least 55% by weight of the therapeutic agent. According to some embodiments, the particles are loaded with an average of at least 60% by weight of the therapeutic agent. According to some embodiments, the particles are loaded with an average of at least 63% by weight of the therapeutic agent. According to some embodiments, the particles are loaded with an average of at least 65% by weight of the therapeutic agent. According to some embodiments, the particles are loaded with an average of at least 70% by weight of the therapeutic agent. According to some embodiments, the particles are loaded with an average of at least 75% by weight of the therapeutic agent. According to some embodiments, the particles are loaded with an average of at least 80% by weight of the therapeutic agent. According to some embodiments, the particles are loaded with an average of at least 85% by weight of the therapeutic agent. According to some embodiments, the particles are loaded with an average of at least 90% by weight of the therapeutic agent. According to some embodiments, the particles are loaded with an average of at least 95% by weight of the therapeutic agent.

Various forms of the therapeutic agent can be used, which are capable of being released from the controlled release system into adjacent tissues or fluids. According to some embodiments, the therapeutic agent can be in liquid or solid form. According to some embodiments, the therapeutic agent is very slightly water soluble, moderately water soluble, or fully water soluble. According to some embodiments, the therapeutic agent can include salts of the API. As such, the therapeutic agent can be an acidic, basic, or amphoteric salt; it can be a nonionic molecule, a polar molecule, or a molecular complex capable of hydrogen bonding; or the therapeutic agent can be included in the compositions in the form of, for example, an uncharged molecule, a molecular complex, a salt, an ether, an ester, an amide, polymer drug conjugate, or other form to provide the effective biological or physiological activity.

Controlled release systems deliver a drug at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

Optimal control is afforded if the drug is placed in a polymeric material or pump. Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing Heller, et al, in Biodegradable Polymers as Drug Delivery Systems, M. Chasin and R. Langer, Eds (Dekker, New York, 1990), pp. 121-161)).

According to some embodiments, the combination of the biodegradable polymers with the therapeutic agent allow a formulation that, when injected or inserted into body, is capable of sustained release of the drug.

According to some embodiments, the therapeutic agent releases from the delivery system through diffusion, conceivably in a biphasic manner. A first phase may involve, for example, a lipophilic drug contained within the lipophilic membrane that diffuses therefrom into an aqueous channel, and the second phase may involve diffusion of the drug from the aqueous channel into the external environment.

According to some embodiments, the microparticulate formulation is characterized by sustained release of the substantially pure polymorphic Form II of nimodipine from the microparticulate formulation such that one half of the polymorphic Form II of nimodipine is released from the microparticulate formulation within 1 day to 30 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 1 day in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 2 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 3 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 4 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 5 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 6 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 7 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 8 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 9 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 10 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 11 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 12 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 13 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 14 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 15 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 16 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 17 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 18 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 19 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 20 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 21 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 22 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 23 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 24 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 25 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 26 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 27 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 28 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 29 days in vivo. According to some embodiments, the microparticulate formulation is characterized by sustained release of the polymorphic Form II of nimodipine from the microparticulate formulation such that one half of the polymorphic Form II of nimodipine is released from the microparticulate formulation within 30 days in vivo.

According to some embodiments, the particulate formulation is presented as a solution. According to some embodiments, the particulate formulation comprises an aqueous solution of the therapeutic agent in water-soluble form. According to some embodiments, the particulate formulation is presented as an emulsion. According to some embodiments, the particulate formulation comprises an oily suspension of the therapeutic agent. An oily suspension of the therapeutic agent can be prepared using suitable lipophilic solvents. Exemplary lipophilic solvents or vehicles include, but are not limited to, fatty oils such as sesame oil, or synthetic fatty acid esters, such as ethyl oleate or triglycerides.

According to some embodiments, the particulate formulation comprises a suspension of particles. According to some embodiments, the suspension of particles comprises a powder suspension of particles. According to some embodiments, the particulate formulation further comprises at least one of a suspending agent, a stabilizing agent and a dispersing agent. According to some embodiments, the particulate formulation comprises an aqueous suspension of the therapeutic agent. Aqueous injection suspensions, for example, can contain substances which increase the viscosity of the suspension, such as sodium carboxymethyl cellulose, sorbitol, hyaluronic acid, or dextran. Optionally, the suspension can also contain suitable stabilizers or agents which increase the solubility of the compounds to allow for the preparation of highly concentrated solutions.

According to some embodiments, the particulate formulation can be in powder form for constitution with a suitable vehicle, e.g., sterile pyrogen-free water, before use. According to some embodiments, the particulate formulation is dispersed in a vehicle to form a dispersion, with the particles as the dispersed phase, and the vehicle as the dispersion medium.

The particulate formulation can include, for example, microencapsulated dosage forms, and if appropriate, with one or more excipients, encochleated, coated onto microscopic gold particles, contained in liposomes, pellets for implantation into the tissue, or dried onto an object to be rubbed into the tissue. As used herein, the term “microencapsulation” refers to a process in which very tiny droplets or particles are surrounded or coated with a continuous film of biocompatible, biodegradable, polymeric or non-polymeric material to produce solid structures including, but not limited to, nonpareils, pellets, crystals, agglomerates, microparticles, or nanoparticles.

Exemplary formulations may be presented in unit-dose or multi-dose containers, for example sealed ampules and vials, and may be stored in a freeze-dried (lyophilized) condition requiring only the addition of the sterile pharmaceutically acceptable carrier, immediately prior to use.

The particulate formulation may be sterilized, for example, by terminal gamma irradiation, e-beam sterilization, filtration through a bacterial-retaining filter or by incorporating sterilizing agents in the form of sterile solid compositions that may be dissolved or dispersed in sterile water or other sterile injectable medium just prior to use. The dose rate is the biggest difference between gamma irradiation and e-beam sterilization. While gamma radiation has a high penetration and a low dose rate, e-beam sterilization has a low penetration and a high dose rate.

Pharmaceutical Compositions

The pharmaceutical compositions of the described invention may take such forms as suspensions, solutions or emulsions in oily or aqueous vehicles, and may contain formulatory agents such as suspending, stabilizing and/or dispersing agents. Suspensions of the active compounds may be prepared as appropriate oily injection suspensions. Exemplary lipophilic solvents or vehicles include fatty oils, synthetic fatty acid esters, or liposomes. Aqueous injection suspensions may contain substances which increase the viscosity of the suspension, such as sodium carboxymethyl cellulose, sorbitol, dextran or hyaluronic acid. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, dichloromethane, acetonitrile, ethyl acetate, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

The pharmaceutical compositions may also contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Exemplary liquid or solid pharmaceutical compositions include, for example, microencapsulated dosage forms, and if appropriate, with one or more excipients, encochleated, coated onto microscopic particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above.

Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol, dichloromethane, ethyl acetate, acetonitrile, etc. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intrathecal, intracerebral, intraventricular, and intraarticular) administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents.

Another method of formulation of the compositions described herein involves conjugating a therapeutic agent of the invention to a polymer that enhances aqueous solubility, including, without limitation, polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. For example, the polymer may be conjugated via an ester linkage to one or more hydroxyls. For example, polyglutamic acids of molecular weights between about 5,000 to about 100,000, between about 20,000 and about 80,000 may be used or between about 30,000 and about 60,000 may be used.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Site-specific activity generally results if the location in the body into which the formulation is deposited is a fluid-filled space or some type of cavity, such as, for example, the subarachnoid space, the subdural cavity of a chronic subdural hematoma or the cavity left after the surgical evacuation of a hematoma, tumor or vascular malformation in the brain. This provides high concentrations of the drug at the site where activity is needed, and lower concentrations in the rest of the body, thus decreasing the risk of unwanted systemic side effects.

Exemplary site-specific delivery systems include use of microparticles (of about 1 μm to about 100 μm in diameter), thermoreversible gels (for example, PGA/PEG), and biodegradable polymers (for example, PLA, PLGA).

According to some embodiments, the delivery characteristics of the therapeutic agent and polymer degradation in vivo can be modified. For example, polymer conjugation can be used to alter the circulation of the drug in the body and to achieve tissue targeting, reduce irritation and improve drug stability. According to some embodiments, the delivery system is a controlled release delivery system. Biodegradable polymeric drug delivery systems that control the release rate of the contained drug in a predetermined manner can overcome practical limitations to targeted delivery. A drug can be attached to soluble macromolecules, such as proteins, polysaccharides, or synthetic polymers via degradable linkages. For example, in animals, antitumor agents such as doxorubicin coupled to N-(2-hydroxypropyl) methacrylamide copolymers showed radically altered pharmacokinetics resulting in reduced toxicity. The half-life of the drug in plasma and the drug levels in the tumor were increased while the concentrations in the periphery decreased. (Kopecek and Duncan, J Controlled Release 6, 315 (1987)). According to some embodiments, polymers, such as polyethylene glycol (PEG), can be attached to drugs to either lengthen their lifetime or alter their immunogenicity; drug longevity and immunogenicity also may be affected by biological approaches, including protein engineering and altering glycosylation patterns. According to some embodiments, polyethylene glycols (PEG's) can be utilized for altering the aqueous component to aid in drug solubilization. Approximately 0.5% to 40% concentration of PEG's (depending on PEG molecular weight) by weight can be placed in approximately 99.5% to 60% H₂O or other aqueous based buffer by weight. Upon heating and stirring, the H₂O (or other aqueous buffer)/PEG combination produces a viscous liquid to a semisolid substance.

According to some embodiments, in order to prolong the effect of a drug, it may be desirable to slow the absorption of the drug. This can be accomplished, for example, by the use of a liquid suspension of crystalline or amorphous material with poor water solubility. The rate of absorption of the drug then depends upon its rate of dissolution which, in turn, may depend upon crystal size and crystalline form. For example, according to some embodiments, a SABER™ Delivery System comprising a high-viscosity base component, is used to provide controlled release of the therapeutic agent. (See U.S. Pat. No. 8,168,217, U.S. Pat. No. 5,747,058 and U.S. Pat. No. 5,968,542, incorporated herein by reference). When the high viscosity SAIB is formulated with drug, biocompatible excipients and other additives, the resulting formulation is liquid enough to inject easily with standard syringes and needles. After injection of a SABER™ formulation, the excipients diffuse away, leaving a viscous depot. SABER™ formulations comprise a drug and a high viscosity liquid carrier material (HVLCM), meaning nonpolymeric, nonwater soluble liquids with a viscosity of at least 5,000 cP at 37° C. that do not crystallize neat under ambient or physiological conditions. HVLCMs may be carbohydrate-based, and may include one or more cyclic carbohydrates chemically combined with one or more carboxylic acids, such as sucrose acetate isobutyrate (SAIB). HVLCMs also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

Additional components can include, without limitation, a rheology modifier, and/or a network former. A rheology modifier is a substance that possesses both a hydrophobic and hydrophilic moiety used to modify viscosity and flow of a liquid formulation, for example, caprylic/capric triglyceride (Migliol 810), isopropyl myristate (IM or IPM), ethyl oleate, triethyl citrate, dimethyl phthalate, and benzyl benzoate. A network former is a compound that forms a network structure when introduced into a liquid medium. Exemplary network formers include cellulose acetate butyrate, carbohydrate polymers, organic acids of carbohydrate polymers and other polymers, hydrogels, as well as particles such as silicon dioxide, ion exchange resins, and/or fiberglass that are capable of associating, aligning or congealing to form three dimensional networks in an aqueous environment.

According to some embodiments, the pharmaceutical composition further comprises a preservative agent.

According to some embodiments, the pharmaceutical composition may further comprise an adjuvant. Exemplary adjuvants include, but are not limited to, preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. Isotonic agents, for example, sugars, sodium chloride and the like, can also be included. Prolonged absorption of an injectable pharmaceutical form can be brought about by the use of agents delaying absorption, for example, aluminum monostearate and gelatin.

### Pharmaceutically Acceptable Carrier

According to some embodiments, the pharmaceutical composition may comprise a pharmaceutically acceptable carrier.

According to some embodiments, the pharmaceutically acceptable carrier is a solid carrier or excipient. According to some embodiments, the pharmaceutically acceptable carrier is a gel-phase carrier or excipient. Examples of carriers or excipients include, but are not limited to, calcium carbonate, calcium phosphate, various monomeric and polymeric sugars (including without limitation hyaluronic acid), starches, cellulose derivatives, gelatin, and polymers. An exemplary carrier can also include a saline vehicle, e.g. hydroxyl propyl methyl cellulose (HPMC) in phosphate buffered saline (PBS).

According to some embodiments, the pharmaceutically acceptable carrier is effective to increase the viscosity of the composition. According to some embodiments, the pharmaceutically acceptable carrier comprises hyaluronic acid. According to some embodiments, the pharmaceutically acceptable carrier comprises 0% to 5% hyaluronic acid. According to some embodiments, the pharmaceutically acceptable carrier comprises less than 0.05% hyaluronic acid. According to some embodiments, the pharmaceutically acceptable carrier comprises less than 0.1% hyaluronic acid. According to some embodiments, the pharmaceutically acceptable carrier comprises less than 0.2% hyaluronic acid. According to some embodiments, the pharmaceutically acceptable carrier comprises less than 0.3% hyaluronic acid. According to some embodiments, the pharmaceutically acceptable carrier comprises less than 0.4% hyaluronic acid. According to some embodiments, the pharmaceutically acceptable carrier comprises less than 0.5% hyaluronic acid. According to some embodiments, the pharmaceutically acceptable carrier comprises less than 0.6% hyaluronic acid. According to some embodiments, the pharmaceutically acceptable carrier comprises less than 0.7% hyaluronic acid. According to some embodiments, the pharmaceutically acceptable carrier comprises less than 0.8% hyaluronic acid. According to some embodiments, the pharmaceutically acceptable carrier comprises less than 0.9% hyaluronic acid. According to some embodiments, the pharmaceutically acceptable carrier comprises less than 1.0% hyaluronic acid. According to some embodiments, the pharmaceutically acceptable carrier comprises less than 1.1% hyaluronic acid. According to some embodiments, the pharmaceutically acceptable carrier comprises less than 1.2% hyaluronic acid. According to some embodiments, the pharmaceutically acceptable carrier comprises less than 1.3% hyaluronic acid. According to some embodiments, the pharmaceutically acceptable carrier comprises less than 1.4% hyaluronic acid. According to some embodiments, the pharmaceutically acceptable carrier comprises less than 1.5% hyaluronic acid. According to some embodiments, the pharmaceutically acceptable carrier comprises less than 1.6% hyaluronic acid. According to some embodiments, the pharmaceutically acceptable carrier comprises less than 1.7% hyaluronic acid. According to some embodiments, the pharmaceutically acceptable carrier comprises less than 1.8% hyaluronic acid. According to some embodiments, the pharmaceutically acceptable carrier comprises less than 1.9% hyaluronic acid. According to some embodiments, the pharmaceutically acceptable carrier comprises less than 2.0% hyaluronic acid. According to some embodiments, the pharmaceutically acceptable carrier comprises less than 2.1% hyaluronic acid. According to some embodiments, the pharmaceutically acceptable carrier comprises less than 2.2% hyaluronic acid. According to some embodiments, the pharmaceutically acceptable carrier comprises less than 2.3% hyaluronic acid. According to some embodiments, the pharmaceutically acceptable carrier comprises less than 2.4% hyaluronic acid. According to some embodiments, the pharmaceutically acceptable carrier comprises less than 2.5% hyaluronic acid. According to some embodiments, the pharmaceutically acceptable carrier comprises less than 2.6% hyaluronic acid. According to some embodiments, the pharmaceutically acceptable carrier comprises less than 2.7% hyaluronic acid. According to some embodiments, the pharmaceutically acceptable carrier comprises less than 2.8% hyaluronic acid. According to some embodiments, the pharmaceutically acceptable carrier comprises less than 2.9% hyaluronic acid. According to some embodiments, the pharmaceutically acceptable carrier comprises less than 3.0% hyaluronic acid. According to some embodiments, the pharmaceutically acceptable carrier comprises less than 3.5% hyaluronic acid. According to some embodiments, the pharmaceutically acceptable carrier comprises less than 4.0% hyaluronic acid. According to some embodiments, the pharmaceutically acceptable carrier comprises less than 4.5% hyaluronic acid. According to some embodiments, the pharmaceutically acceptable carrier comprises less than 5.0% hyaluronic acid.

According to some embodiments, the pharmaceutically acceptable carrier comprises a gel compound. According to some embodiments, the gel compound is a biodegradable hydrogel. For example, glyceryl monooleate (GMO) provides a predominantly lipid-based hydrogel, which has the ability to incorporate lipophilic materials, with internal aqueous channels that incorporate and deliver hydrophilic compounds. It is recognized that at room temperature (25° C.), the gel system may exhibit differing phases which comprise a broad range of viscosity measures.

According to some embodiments, a GMO hydrogel delivery system can be produced by heating GMO above its melting point (40-50° C.) and by adding a warm aqueous-based buffer or electrolyte solution, such as, for example, phosphate buffer or normal saline, which thus produces a three-dimensional structure. The aqueous-based buffer may be comprised of other aqueous solutions or combinations containing semi-polar solvents.

According to some embodiments, two gel system phases may be utilized due to their properties at room temperature and physiologic temperature (about 37° C.) and pH (about 7.4). According to some embodiments, for GMO, within the two gel system phases, the first phase is a lamellar phase of approximately 5% to approximately 15% H₂O content comprising a moderately viscous fluid that may be easily manipulated, poured and injected, and approximately 95% to approximately 85% GMO content. The second phase is a cubic phase containing approximately 15% to approximately 40% H₂O content and approximately 85%-60% GMO content, with an equilibrium water content of approximately 35% to approximately 40% by weight. The term “equilibrium water content” as used herein refers to maximum water content in the presence of excess water. Thus the cubic phase incorporates water at approximately 35% to approximately 40% by weight. The cubic phase is highly viscous. According to some embodiments, the viscosity exceeds 1.2 million centipoise (cP) when measured by a Brookfield viscometer; where 1.2 million cP is the maximum measure of viscosity obtainable via the cup and bob configuration of the Brookfield viscometer.

Alternatively, according to some embodiments, modified formulations and methods of production are utilized such that the nature of the delivery system is altered, or in the alternative, aqueous channels contained within the delivery system are altered. Thus, various therapeutic agents in varying concentrations may diffuse from the delivery system at differing rates, or be released therefrom over time via the aqueous channels of the delivery system. Hydrophilic substances may be utilized to alter the consistency or therapeutic agent release by alteration of viscosity, fluidity, surface tension or the polarity of the aqueous component.

For example, glyceryl monostearate (GMS), which is structurally identical to GMO with the exception of a double bond at Carbon 9 and Carbon 10 of the fatty acid moiety rather than a single bond, does not gel upon heating and the addition of an aqueous component, as does GMO. However, because GMS is a surfactant, GMS is miscible in water up to approximately 20% weight/weight. The term “surfactant” as used herein refers to a surface active agent that is miscible in water in limited concentrations as well as polar substances. Upon heating and stirring, the 80% H₂O/20% GMS combination produces a spreadable paste having a consistency resembling hand lotion. The paste then is combined with melted GMO so as to form the cubic phase gel possessing a high viscosity referred to above.

According to some embodiments, a hydrolyzed gelatin, such as commercially available Gelfoam™, can be utilized for altering the aqueous component. Approximately 6.25% to 12.50% concentration of Gelfoam™ by weight may be placed in approximately 93.75% to 87.50% respectively by weight H₂O or another aqueous based buffer. Upon heating and stirring, the H₂O (or other aqueous buffer)/Gelfoam™combination produces a thick gelatinous substance. The resulting substance is combined with GMO; a product so formed swells and forms a highly viscous, translucent gel being less malleable in comparison to neat GMO gel alone.

According to some embodiments, the therapeutic agent releases from the delivery system through diffusion. According to some embodiments, the therapeutic agent releases from the delivery system through diffusion in a biphasic manner. A first phase may involve, for example, a lipophilic drug contained within the lipophilic membrane that diffuses therefrom into an aqueous channel, and the second phase may involve diffusion of the drug from the aqueous channel into the external environment. For example, being lipophilic, the drug may orient itself inside the GMO gel within its proposed lipid bi-layer structure. Thus, incorporating greater than approximately 7.5% of the drug by weight into GMO causes a loss of the integrity of the three-dimensional structure whereby the gel system no longer maintains the semisolid cubic phase, and reverts to the viscous lamellar phase liquid. According to some embodiments, about 1% to about 45% of therapeutic agent is incorporated by weight into a GMO gel at physiologic temperature without disruption of the normal three-dimensional structure. As a result, this system can allow for increased flexibility with drug dosages.

Alternatively, the described invention may provide a delivery system, which acts as a vehicle for local delivery of substantially pure polymorphic Form II of nimodipine comprising a lipophilic, hydrophilic or amphophilic, solid or semisolid substance, heated above its melting point and thereafter followed by inclusion of a warm aqueous component so as to produce a gelatinous composition of variable viscosity based on water content. Therapeutic agent(s) is/are incorporated and dispersed into the melted lipophilic component or the aqueous buffer component prior to mixing and formation of the semisolid system. The gelatinous composition is placed within the semisolid delivery apparatus for subsequent placement, or deposition.

Process

According to some embodiments, a scalable process for manufacturing a microparticulate formulation comprising a substantially pure polymorphic Form II of nimodipine comprises providing an API starting material containing at least 70% polymorphic Form I of nimodipine. According to some such embodiments, the process for producing nimodipine Form II containing microparticles from the nimodipine Form I API starting material comprises:

(1) providing an API starting material containing a substantially pure polymorphic Form I of nimodipine;

(2) adding the API starting material of (1) to a polymer solution, thereby forming polymorphic Form II of nimodipine and creating a mixture of the polymorphic Form II of nimodipine and the polymer solution;

(3) homogenizing the mixture of (2) to form a disperse phase comprising the nimodipine;

(4) providing a continuous phase in which the dispersed phase will form an emulsion;

(5) introducing the dispersed phase and continuous phase into a reactor vessel, the reactor vessel including a continuous process medium, thereby forming an emulsion of the dispersed phase in the continuous phase comprising the nimodipine;

(6) causing the polymer to form microparticles containing polymorphic Form II of nimodipine;

(7) transporting the emulsion from the reactor vessel to a solvent removal vessel and removing the solvent;

(8) formulating the nimodipine Form II-containing microparticles by: (i) maintaining a suspension of nimodipine Form II-containing microparticles in the continuous phase; and (ii) washing the nimodipine Form II-containing microparticles; and

(9) drying the nimodipine Form II-containing microparticles.

According to some embodiments the API starting material is milled. According to some embodiments, the API starting material is unmilled.

According to some embodiments, the washing step is conducted by replacing the continuous phase with water by moving the suspension through a filter adapted to remove continuous phase and return the microparticles to a process vessel while maintaining the suspension; replacing the water with a formulating medium by moving the suspension through a filter adapted to eliminate the water and return the microparticles to a process vessel while maintaining the microparticles in suspension; and removing the suspension of microparticles containing the bioactive agent and formulating medium from the process vessel. According to some embodiments, the washing step is conducted by moving the suspension through a hollow fiber filter.

According to some embodiments, where a polymer solution comprises a polymer in an organic solvent forming an oil/water emulsion in the disperse phase, mixing the disperse phase with the continuous phase results in a double emulsion (i.e., a water/oil/water emulsion). According to some embodiments, where the polymer solution comprises a polymer in an aqueous solvent such as water, only a single emulsion is formed upon mixing the dispersed phase with the continuous phase.

According to some embodiments, the continuous process medium comprises a surfactant and the nimodipine saturated with the solvent used in the polymer solution.

Exemplary solvents include “halogenated solvents” and “non-halogenated solvents.” Non-limiting examples of non-halogenated solvents include: dimethylsulfoxide (DMSO), triacetin, N-methylpyrrolidone (NMP), 2-pyrrolidone, dimethylformamide (DMF), miglyol, isopropyl myristate, triethyl citrate, propylene glycol, ethyl carbonate, ethyl acetate, ethyl formate, methyl acetate, glacial acetic acid, polyethylene glycol (200), polyethylene glycol (400), acetone, methyl ethyl ketone, methanol, ethanol, n-propanol, iso-propanol, benzyl alcohol, glycerol, diethyl ether, tetrahydrofuran, glyme, diglyme, n-pentane, iso-pentane, hexane, heptane, isooctane, benzene, toluene, xylene (all isomers), and the like. Non-limiting examples of halogenated solvents include carbon tetrachloride, chloroform, methylene chloride (i.e., dicholoro methane, DCM), chloroethane, 1,1-dichloroethane, 1,1,1-trichloroethane, and 1,2-dichloroethane.

According to some embodiments, the polymer solution can comprise nimodipine and a solvent such as, for example, ethyl acetate or methylene chloride. Depending on the polymer in use, a movement from dichloromethane to ethyl acetate can increase the purity of the end product.

According to some embodiments, the microparticles can be dried by any conventional means known in the art. According to some embodiments, the microparticles can be dried via lyophilization. According to some embodiments, the microparticles can be dried under nitrogen flow. Typically lyophilization is a fast drying process whereas nitrogen flow is a slower rate process, but can be varied. For example, drying time can be from 4 to 12 hours, from 4 to 16 hours, from 4 to 24 hours, from 4 to 48 hours, from 4 to 60 hours, from 12 to 14 hours, from 16 to 24 hours, or from 24 to 48 hours.

During crystallization of the form II of nimodipine formed in situ from the nimodipine Form I API starting material, particle size may be difficult to control, and may result in large particles. For example, according to some embodiments, the distribution of particle size can be from 20 μm to 250 μm. According to some embodiments, the mean particle size (D50) ranges from 35 μm to 227 μm, i.e., including 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, 160 μm, 165 μm, 170 μm, 175 μm, 180 μm, 185 μm, 190 μm, 195 μm, 200 μm, 205 μm, 210 μm, 215 μm, 220 μm, 221 μm, 222 μm, 223 μm, 224 μm, 225 μm, 226 μm, and 227 μm. According to some embodiments, microparticles produced by this process are characterized by D10>10 μm, mean particle size (D50) 70-80 μm, and D90<200 μm.

According to some embodiments, an alternate scalable process for manufacturing a microparticulate formulation comprising a substantially pure polymorphic Form II of nimodipine comprises providing an API starting material containing polymorphic Form II of nimodipine. According to some embodiments, the process for manufacturing nimodipine Form II-containing microparticles from the nimodipine Form II API starting material comprises:

(1) preparing an API starting material containing substantially pure nimodipine Form II by:
(a) synthesizing an API starting material containing substantially pure polymorphic Form II of nimodipine; or
(b) crystallizing Form II of nimodipine from Form I by dissolving Form I of nimodipine in a solvent and evaporating the solvent to yield Form II;

(2) completing the disperse phase by adding the API starting material of step (1) to a polymer solution, thereby creating a mixture of polymorphic Form II of nimodipine and the polymer solution in ethyl acetate (solvent);

(3) homogenizing the continuous phase comprising polyvinyl alcohol (PVA) in water with the dispersed phase of step (2) to form an emulsion;

(4) introducing a water stream continuously post-microparticle formation, causing the polymer to form nimodipine Form II-containing microparticles;

(5) transporting the emulsion from the reactor vessel to a solvent removal vessel and removing the solvent;

6) formulating the Form II containing microparticles by
(i) maintaining a suspension of the Form II containing microparticles in the continuous phase;
(ii) washing the Form II containing microparticles; and

(7) drying the Form II containing microparticles.

According to some embodiments the API starting material is milled, micronized or both. According to some embodiments, the API starting material is unmilled.

According to some embodiments, the washing is conducted by replacing the continuous phase containing ethyl acetate with water by moving the suspension through a filter adapted to remove the continuous phase and return the microparticles to a process vessel while maintaining the suspension and removing the suspension of microparticles containing the bioactive agent and formulating medium from the process vessel. According to some embodiments, the washing is conducted by moving the suspension through a hollow fiber filter.

According to some embodiments, this process allows for better control of particle size and better yield than the process with nimodipine form I as the API starting material. According to some embodiments, microparticles manufactured according to this process with milled and micronized substantially pure polymorphic Form II of nimodipine as the API starting material are characterized by D10>2 μm, D50 is about 7 μm and D90 is <10 μm.

According to some embodiments, the microparticulate suspension comprising the polymorphic Form II of nimodipine is light stable. According to some embodiments, the microparticulate suspension comprising the polymorphic Form II of nimodipine is chemically stable.

According to some embodiments, entrapment efficiency, meaning the percentage of drug retained by the microparticles relative to the total amount available is about 95%.

According to some embodiments, the microparticulate suspension is characterized by a drug load of nimodipine of at least 55%, at least 56%, at least 57%, at least 58%, at least 59%, at least 60%, at least 61%, at least 62%, at least 63%, at least 64%, or at least 65% by weight relative to the total weight of the formulation.

According to some embodiments, the polymer concentration ranges from about 14% to about 30%, i.e., the polymer concentration is 14%, 15%, 16%, 17%, 18%, 19%, 20%, 21%, 22%, 23%, 24%, 25%, 26%, 27%, 28%, 29%, or 30%. According to some embodiments, the microparticles comprise a poly (lactide-co-glycolide) polymer matrix. According to some embodiments, the lactide to glycolide ratio of the poly (lactide-co-glycolide) is 50:50. According to some embodiments inherent viscosity of the polymer is at least 0.16 dl/g, at least 0.17 dl/g, at least 0.18 dl/g, at least 0.19 dl/g, at least 0.20 dl/g, at least 0.21 dl/g, at least 0.22 dl/g, at least 0.23 dl/g, or at least 0.24 dl/g. According to some embodiments, molecular weight of the polymer is at least 20 kDa, at least 21 kDa, at least 22 kDa, at least 23 kDa, at least 24 kDa, at least 25 kDa, at least 26 kDa, at least 27 kDa, or at least 28 kDa.

According to some embodiments, the polymorphic Form II of nimodipine is dispersed throughout the polymer matrix. According to some embodiments, the polymer matrix is impregnated with the polymorphic Form II of nimodipine.

According to some embodiments, the polymorphic Form II of nimodipine includes less than 20% by weight of any other physical forms of nimodipine. According to some embodiments the microparticulate formulation contains less than 10% of Form I of nimodipine. According to some embodiments the microparticulate formulation is substantially free of Form I of nimodipine.

According to some embodiments, the microparticulate formulation displays delayed release kinetics, such that one half of the polymorphic Form II of nimodipine is released within 1 day to 30 days in vitro.

Use in the Preparation of a Medicament

According to another aspect, the described invention provides use of a pharmaceutical composition formulated for delivery by injection containing a microparticulate formulation comprising a microparticle suspension comprising a therapeutic amount of substantially pure Form II of nimodipine characterized by an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both, and a pharmaceutically acceptable carrier comprising an agent that affects viscosity of the microparticulate suspension in the manufacture of a medicament for reducing severity or incidence of a delayed complication associated with a brain injury including interruption of a cerebral artery that deposits blood in a subarachnoid space, wherein the delayed complication is selected from the group consisting of a microthromboembolism, a delayed cerebral ischemia (DCI) caused by formation one or more of microthromboemboli, or cortical spreading ischemia (CSI) and a cortical spreading ischemia (CSI), wherein the brain injury is mediated by decreased cerebral perfusion in a human subject.

According to another aspect, the described invention provides a method for reducing severity or incidence of a delayed complication associated with a brain injury including interruption of a cerebral artery that deposits blood in a subarachnoid space, wherein the delayed complication is selected from the group consisting of a microthromboembolism, a delayed cerebral ischemia (DCI) caused by formation one or more of microthromboemboli, or cortical spreading ischemia (CSI) and a cortical spreading ischemia (CSI), comprising: (a) providing a microparticulate formulation comprising a microparticle suspension comprising a therapeutic amount of substantially pure polymorphic Form II of nimodipine characterized by an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both, and a pharmaceutically acceptable carrier comprising an agent that affects viscosity of the microparticulate suspension, wherein the particles comprise a poly (lactide-co-glycolide) polymer matrix; and (ii) a pharmaceutically acceptable carrier comprising an agent that affects viscosity of the microparticulate suspension. The microparticulate formulation is formulated for delivery locally, either (i) into a cerebral ventricle, (ii) intracisternally into the subarachnoid space in a subarachnoid cistern closest to a cerebral artery at risk for interruption; or (iii) intrathecally. The microparticulate suspension is characterized by gradual release of the polymorphic Form II of nimodipine from the microparticle suspension over an extended period of time. The therapeutic amount is effective to bathe the arteries on the outside of the brain, to open these small arteries over the surface of the brain, and to decrease delayed cerebral ischemia due to angiographic vasospasm, cortical spreading ischemia, microthromboemboli or a combination by improving cerebral perfusion, thereby treating the delayed complication, without risk of systemic hypotension, pulmonary vasodilation, pulmonary edema, and lung injury.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Preparation of Microparticles Containing Substantially Pure Polymorphic Form II of Nimodipine

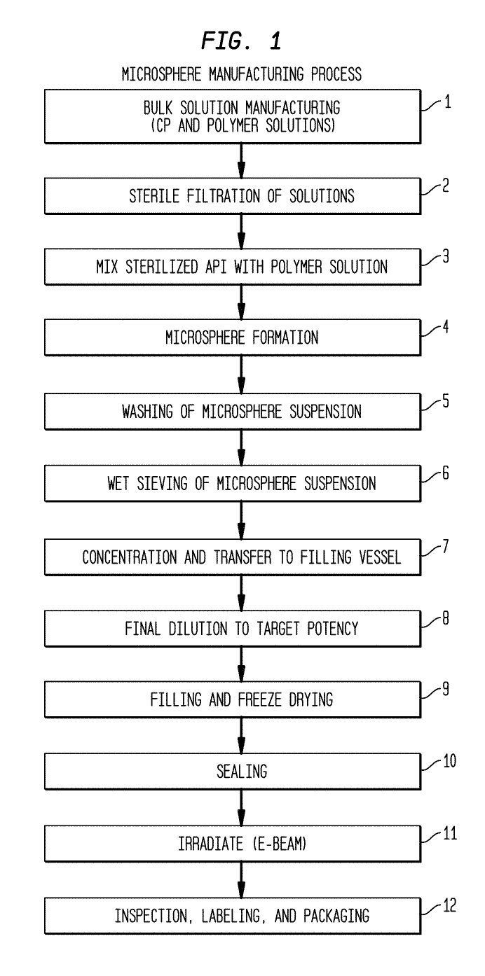

The overall process for making the nimodipine microparticles of the described invention is shown in FIG. 1.

### Step 1. Bulk Solution Manufacturing

The Continuous Phase (CP) consisting of 0.035 g/g polyvinyl alcohol (PVA) in water is produced by dispersing PVA powder in ambient temperature water for injection (WFI), heating to at least 70° C. while mixing to dissolve the powder, cooling the solution to ambient temperature, and bringing the solution to its final weight with WFI.

Polymer solution is prepared by combining a mixture of PLGA dissolved in ethyl acetate at a concentration of 0.22 g/g.

Water for Injection (WFI) is collected on demand for washing of the microparticles at 25° C.

### Step 2. Sterile Filtration of Solutions

The two bulk solutions (i.e., the Continuous Phase and the Polymer Solution) are filtered into the sterile core using 0.22 μm filters. Common membrane types ae shown in Table 1. The CP is sterile filtered directly into the in-line mixer. WFI is sterile filtered directly into the solvent removal vessel (SRV) that collects microparticles as they exit the in-line mixer. The polymer solution is sterile filtered directly into the vessel containing the sterilized API powder.

TABLE 1
Sterilizing Filters for Bulk Solutions

| Solution | Sterilizing Filter |
|---|---|
| Continuous Phase | Hydrophilic PVDF |
| Polymer Solution Phase | Hydrophobic PTFE |
| Water for Injection | Hydrophilic PVDF |

Step 3. Mix Sterilized API Powder with Polymer Solution

The dispersed phase (DP) consists of the polymer, nimodipine and ethyl acetate. The polymer solution is first prepared by dissolving the polymer in ethyl acetate with stirring. After polymer dissolution, the nimodipine powder is pre-weighed into a glass vessel and sterilized by irradiation. The vessel containing sterilized API is connected to the process equipment aseptically. A specified weight of polymer solution is sterile filtered directly into the sterilized API vessel and mixed on a stir-plate until complete wetting and a homogeneous suspension is obtained. This suspension is then transferred into a top-stirred vessel to obtain a homogeneous dispersed (DP) suspension.

### Step 4. Microparticle Formation

Microparticles are formed by combining the CP and DP in a high-shear in-line mixer. The microparticle suspension produced in the high shear mixer is received into the SRV along with sterile filtered ambient WFI. The suspension in the SRV is continuously re-circulated through a hollow fiber filter (HFF, 0.45 micron cut-off membrane, e.g. GE Healthcare Products CFP-4-E-35A) and the filtrate is removed at the combined rate of suspension addition and WFI addition to the SRV. Thus, a constant volume of suspension in maintained in the SRV during the formation stage. Microparticle formation parameters are shown in Table 2.

TABLE 2
Microparticle Formation Parameters for scaled-up batch size

| Process Step | Parameters | Value | |
|---|---|---|---|
| Microparticle | Dispersed phase flow rate | 75 | ml/min |
| Formation | Continuous phase flow rate | 3 | L/min |
| Microparticle | In-line mixing speed | 3000-3300 | RPM |
| Concentration in SRV | Water addition rate | 4 | L/min |
| | HFF Re-circulation rate | 33 | L/min |
| | Filtrate removal rate | 7 | L/min |

### Step 5. Washing of Microparticle Suspension

After the microparticle formation step is complete, ambient water is added while removing the suspending medium in the SRV using the HFF to remove PVA, ethyl acetate and any unencapsulated nimodipine. A sufficient number of volume exchanges are performed to reduce residual PVA and to reduce the residual solvent levels below the ICH/USP limit.

### Step 6. Wet Sieving of Microparticle Suspension

At the end of the washing step, the microparticle suspension is re-circulated through a sieve bag (250 NMO) to remove large microparticles and any non-spherical precipitates. The microparticle process is developed such that there will be very little polymer that precipitates into non-spherical particles.

### Step 7. Concentration and Transfer to Filling Vessel

The volume of the washed and sieved microparticle suspension is reduced by using the HFF to remove a portion of the WFI serving as the suspending medium. Because no microparticles are removed by the filter, the microparticle/nimodipine concentration increases. The volume of WFI removed is sufficient so that the microparticle concentration will be greater than the target for filling. The concentrated suspension is transferred to a second sterile vessel on a tared balance, and an in-process suspension sample is collected and its nimodipine concentration measured.

### Step 8. Final Dilution to Target Potency

A precise amount of WFI is added to the filling vessel to reduce the concentration to its target for filling. A second in-process sample is collected to confirm that the dilution was performed correctly.

### Step 9-10. Filling to Sealing

The microparticle suspension is filled using a filler equipped with a peristaltic pump to aseptically dispense product so that there is no solution contact with the pumping system. Routine fill volume checks are performed throughout the process to ensure compliance with fill volume requirements.

The filled drug product is loaded into the lyophilization chamber onto pre-chilled shelves. Once the cycle is complete the stoppers are fully seated with a nitrogen headspace in the vials, the chamber is unloaded, and the vials are transferred directly to the inner sealing turntable for sealing/capping.

The fully stoppered vials are conveyed to the capper and sealed. Tray segregation is maintained by only capping one tray at a time. A new tray of vials is not loaded onto the in-feed turntable until the previous tray is completely sealed and trayed. The trays of vials are stacked onto pallets, wrapped and then transferred to the controlled room temperature storage area.

### Step 11. Irradiate (E-Beam)

The vials are irradiated via e-beam, using validated conditions.

### 12. Inspection, Labeling, and Packaging

Final Product Samples are obtained and tested per the requirements of the Final Product Specification.

The finished lot is transferred to the inspection area. Rejects are culled while maintaining tray segregation. Rejects include glass defects (cracks or chips), crimp defects (missing flap cap, loose seal, and damaged crimp), and product defects (discoloration, low or high fills, glass or metal present, and other foreign matter).

### Example 2. Dispersed Phase Parameters

The dispersed phase (DP) consists of the polymer, nimodipine and ethyl acetate. The polymer is completely dissolved and the nimodipine drug powder is only partially dissolved.

The DP (125 g scale) was prepared in a closed 1 liter Applikon with top stirrer. The polymer solution was first prepared by dissolving the polymer in ethyl acetate that was stirring inside the 1 liter Applikon. After polymer dissolution, the nimodipine powder (65% target load) was weighed out separately and then added to the stirring polymer solution. After a homogeneous suspension was achieved (and recorded), In process samples of the DP were taken for determination of nimodipine concentration and for nimodipine particle settling rate measurement. After the in-process samples were taken, a portion of the DP was used to prepare microparticles.

The target drug load (65% nimodipine), temperature of the solution and mixing speed were held constant, while the molecular weight of the polymer (a high and low), the nimodipine particle size (milled and un-milled) and the polymer concentration in the ethyl acetate (a low and high) were varied as shown in Table 3, and the effect of these variables on the nimodipine concentration, the settling rate of the nimodipine particles, and on the characteristics of the final microparticle was determined.

TABLE 3
DP variables

| Variable | L1 | L2 |
|---|---|---|
| Polymer molecular weight or IV | 28 kDa | 52 kDa |
| | (IV = 0.24 dl/g) | (IV = 0.38 dl/g) |
| Polymer concentration | 10% | 30% |
| Nimodipine particle form | Unmilled | Milled |

The CP flow rate was 2 L/min, the DP flow rate was 25 ml/min, and a microparticle formation speed of 2000 RPM for the homogenizer. The water addition rate was 2 L/min. Using the 30 L jacketed stainless steel tank, the microparticle suspension was introduced for approximately 7 minutes or until almost full (˜25-30 Liters). The DP, CP and water flow were then turned off.

As soon as microparticle formation was complete, the suspension was transferred to the 5 L Applikon where room temperature water washing was performed at 0.35 L/min for 150 min, and ˜3 L suspension will be maintained. Bulk microparticles were collected on a 5 μm filter and freeze-dried.

Results

The expected API concentration in the DP was 144.4 and 299.8 mg/g for the 10 and 30% polymer concentration, respectively. Measured values were within 4% of the target. The separation volume was a measure of the settling rate of the drug particles within the DP, and was highly dependent on the viscosity of the DP. In general, the higher the polymer concentration, the higher the solution viscosity, and the lower the separation volume. In addition, the higher molecular weight of the polymer produced less separation, with all other parameters the same. In most cases, the larger un-milled nimodipine particles settled at a faster rate (larger separation volume) due to their larger size.

These batches were taken to microparticle completion including washing and freeze-drying. The characteristics of the formed microparticles is shown in Table 4.

TABLE 4

| | | | Mw | | | | | |
|---|---|---|---|---|---|---|---|---|
| | | | Polymer | | | | | In vitro |
| | | Nimodipine | (kDa) (IV | Polymer | Drug | Microparticle | Residual | release |
| | | Particle | Polymer | Concentration | Load | Size (d50; | solvent | at 24 hr |
| Experiment | Batch | Form | dl/g) | (wt %) | (wt %) | μm) | (wt %) | (5) |
| 1 | A | Milled | 28 (0.24) | 10 | 64.3 | 40 | 0.7 | 36.9 |
| 2 | B | Milled | 28 (0.24) | 30 | 64.7 | 175 | 0.7 | 26.7 |
| 3 | C | Milled | 40 (0.38) | 10 | 64.2 | 49 | 1.1 | 19.5 |
| 4 | D | Milled | 40 (0.38) | 30 | 64.9 | 227 | 0.7 | 15.9 |
| 5 | E | Un-Milled | 28 (0.24) | 10 | 64.7 | 35 | 0.6 | 30.8 |
| 6 | F | Un-Milled | 28 (0.24) | 30 | 62.7 | 146 | 1.1 | 9.6 |
| 7 | G | Un-Milled | 40 (0.38) | 10 | 63.8 | 57 | 2.2 | 12.6 |

The drug load was similar for all of the batches, regardless of nimodipine particle size and polymer molecular weight and concentration. The microparticle size was highly dependent on the polymer concentration which is related to the viscosity of the DP solution. For the same homogenizer speed, higher polymer molecular weight and concentration produced larger microparticles, with polymer concentration showing the greatest effect. The size of nimodipine drug particles had less of an effect on final microparticle size. Residual solvent ranged from 0.6-2.2%, with no clear trend among the test variables. The initial in vitro release at 24 hr determined by a shaker bath method is also given in Table 3. In general, the lower polymer molecular weight and concentration released faster.

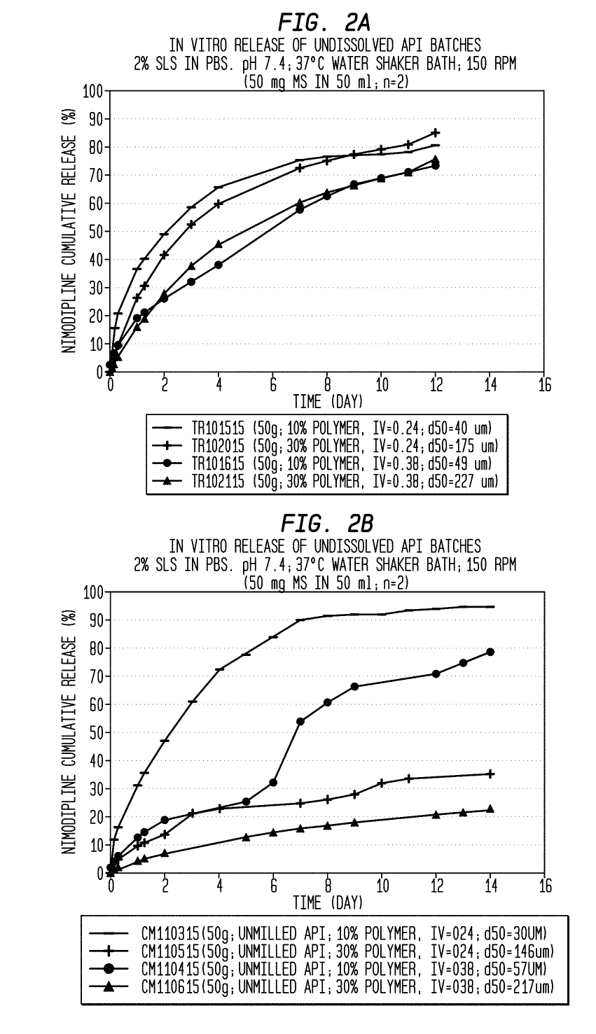

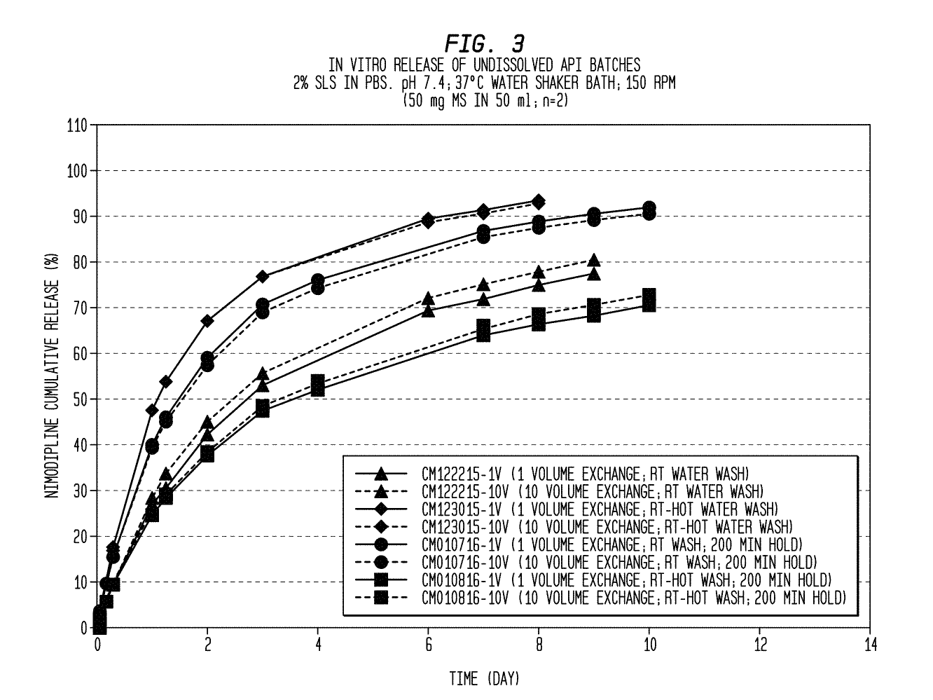

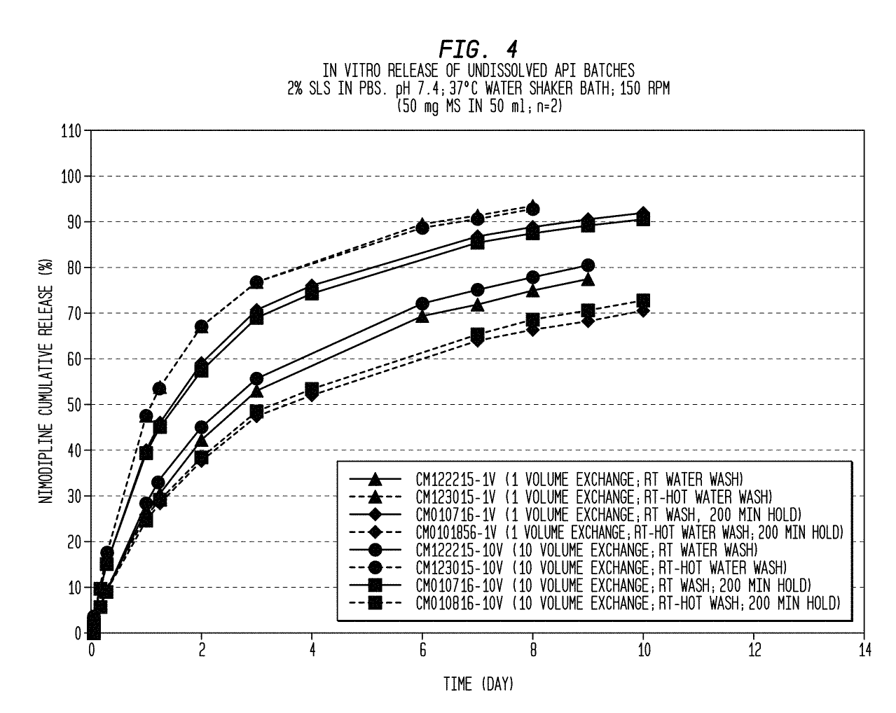

The complete release profiles are shown in FIGS. 2A (milled nimodipine) and 2B (un-milled nimodipine). There were larger differences among the un-milled formulations compared to the milled formulations.

### Example 4. Formation Phase Parameters

Polymer molecular weight and polymer concentration in the DP was held constant. The polymer concentration was 22%, based on the DP results.

The effect of DP flow rate, CP flow rate, mixer speed and water dilution flow rate on the freshly-formed microparticle size and drug load, as well as the solvent concentration in the external phase of the suspension, was measured. API RD2277 of RD2277 (Lusochimica; lot NIM115), a polymer concentration of 22% and molecular weight of 40 kDa (IV=0.38 dl/g) was used for all studies.

A bulk DP solution was prepared for multiple experiments to be performed during a 1 day experiment. The polymer solution was prepared in a closed 1 liter Applikon with top stirrer, by adding the solvent first and then adding the pre-weighed polymer where it was stirred until dissolution.

The DP was prepared by adding the pre-weighed drug powder to the polymer solution in the Applikon. The stirring continued until the nimodipine particles were homogeneously dispersed throughout the DP.

The DP flow rate, the CP flow rate, mixer speed and water dilution were varied according to Table 5.

TABLE 5

| Variable | L1 | L2 |
|---|---|---|
| Dispersed Phase Flow Rate (ml/min) | 12.5 | 37.5 |
| Continuous Phase Flow Rate (ml/min) | 1000 | 3000 |
| Mixer Speed (RPM) | 1400 | 3000 |
| Water Dilution Flow Rate (ml/min) | 1000 | 3000 |

For each set of variables, the formation step was allowed to continue for a short time (i.e. ˜2 min) to achieve equilibrium and then the freshly-formed microspheres were sampled into a 2 liter bottle.

A portion of each suspension was collected via vacuum filtration and washed with excess water and the particle size was immediately measured using the R&D particle sizer. In addition, a portion of the collected filtrate was freeze-dried for an in-process drug load measurement.

Finally, a portion of each suspension was filtered into a GC vial via syringe disc filter to collect the external continuous phase for ethyl acetate concentration.

The API lot of RD2277 (Lusochimica; lot NIM115), a polymer concentration of 22% and molecular weight of 40 kDa (IV=0.38 dl/g) was used for all studies.

Results are shown in Table 6.

TABLE 6
Formation Parameter Results

| | | | | | | Water | | | |
|---|---|---|---|---|---|---|---|---|---|
| | | | | | | Dilution | Drug | | Ethyl acetate |
| | | | DP flow | CP flow | Homogenizer | Flow | Load | | concentration |
| | | | rate | rate | mixer speed | Rate | (wt | D50 | in external |
| Experiment | Batch | CP/DP | (ml/min) | (ml/min) | (RPM) | (ml/min) | %) | (μm) | phase (ppm) |
| 1 | 1 | 80 | 12.5 | 1000 | 1400 | 1000 | 64.2 | 288 | 4221 |
| 2 | 2 | 80 | 12.5 | 1000 | 1400 | 3000 | 64.3 | 254 | 2636 |
| 3 | 3 | 240 | 12.5 | 3000 | 1400 | 1000 | 64.3 | 271 | 2134 |
| 4 | 4 | 240 | 12.5 | 3000 | 1400 | 3000 | 64.3 | 270 | 1572 |
| 5 | 5 | 80 | 12.5 | 1000 | 3000 | 1000 | 64.6 | 124 | 4566 |
| 6 | 6 | 80 | 12.5 | 1000 | 3000 | 3000 | 64.4 | 139 | 2459 |
| 7 | 7 | 240 | 12.5 | 3000 | 3000 | 1000 | 64.6 | 129 | 2151 |
| 8 | 8 | 240 | 12.5 | 3000 | 3000 | 3000 | 64.8 | 136 | 1624 |
| 9 | 9 | 27 | 37.5 | 1000 | 1400 | 1000 | 64.3 | 293 | 12288 |
| 10 | 10 | 27 | 37.5 | 1000 | 1400 | 3000 | 64.3 | 293 | 7975 |
| 11 | 11 | 80 | 37.5 | 3000 | 1400 | 1000 | 64.4 | 278 | 5807 |
| 12 | 12 | 80 | 37.5 | 3000 | 1400 | 3000 | 64.4 | 252 | 4573 |
| 13 | 13 | 27 | 37.5 | 1000 | 3000 | 1000 | 64.9 | 121 | 12091 |
| 14 | 14 | 27 | 37.5 | 1000 | 3000 | 3000 | 64.6 | 121 | 8143 |
| 15 | 15 | 80 | 37.5 | 3000 | 3000 | 1000 | 64.9 | 131 | 5666 |
| 16 | 16 | 80 | 37.5 | 3000 | 3000 | 3000 | 64.7 | 134 | 4580 |

The drug load of the freshly-formed microparticles was similar for all parameter configurations, which indicates that the drug load is not dependent on the CP/DP ratio, mixing speed or the dilution rate. Nimodipine has very low water solubility, and drug losses to the external aqueous phase during the formation step are negligible.

The average size of the freshly-formed microparticles was dependent on the homogenizer mixing speed, with higher mixing speeds producing smaller microparticles.

Finally, the amount of ethyl acetate in the external phase was determined by the CP/DP ratio and the level of water dilution. The highest solvent concentrations were observed for the higher DP flow rate of 37.5 ml/min compared to its 12.5 ml/min counterpart. In addition, highest solvent concentrations were measured for the lower water dilution rate of 1000 ml/min compared to its 3000 ml/min counterpart, which was expected.

### Example 5. Solvent Removal Parameters

The effect of the number of volume exchanges, the temperature and temperature profile of the washing water cycle and the hold time of the suspension prior to washing on the microparticle size, drug load, residual solvent and the in vitro release was determined.

The polymer solution was prepared in a closed 1 liter Applikon with top stirrer, by adding the solvent first and then adding the pre-weighed polymer where it was stirred until dissolution.

The dispersed phase (DP) was prepared by adding the pre-weighed drug powder to the polymer solution in the Applikon. The stirring continued until the nimodipine particles were homogeneously dispersed throughout the DP.

The continuous phase (CP) solution contained 0.35% polyvinyl alcohol.

The DP flow rate, the CP flow rate, mixer speed and water dilution flow rate were set according to Table 5.

A 20 Liter glass solvent removal vessel (SRV) received the freshly-formed microsphere suspension, and was concentrated to ˜15 Liters during the microsphere formation step using hollow fiber filter recirculation and permeate removal.

As soon as microsphere formation was complete, either water washing or the hold time was initiated. During the holding step, the suspension was stirred in the SRV and slowly recirculated through the HFF with all other ports closed.

Washing Temperature of 25° C.: After 1 volume exchange, 2 liters of suspension were removed and collected on a filter (Amicon) and placed in stainless steel cup/tray for lyophilization. The remaining suspension continued to be washed until 10 volume exchanges was completed. The microspheres were then collected on a filter (Amicon) and place in stainless steel cup/tray for lyophilization.

Washing Temperature of 25-35-25° C.: After microsphere formation or hold time was complete, 2 liters of suspension was transferred into a 3 L Applikon/stirrer connected to a small HFF. The washing cycle was started according to the following table for 1 volume exchange for this portion of suspension. For the remaining suspension in the 20 L SRV, 10 volume exchanges were performed at 2 L/min according to the following table:

TABLE 7
Washing Temperature Cycle

| Washing | Time at each temperature during washing (min) | |
|---|---|---|
| Temperature | 1 Volume Exchanges @ | 10 Volume Exchanges @ |
| (° C.) | 250 ml/min | 2 L/min |
| 25 | 3 | 30 |
| 25-35 ramp | 1 | 10 |
| ≥35 hold | 3 | 30 |
| ≤27 cool | 1 | 10 |

After the washing steps were completed, each portion of microspheres was collected on a filter (Amicon) and placed in a stainless steel cup/tray for lyophilization.

Results

The drug load of the lyophilized microparticles was similar for all parameter configurations, which indicates that the drug load is not dependent on the extent of washing and washing temperatures or hold time.

In general, microparticle size was not affected by the extent of washing and its temperature cycle.

As expected, the amount of volume exchanges affected the residual solvent within the microparticles. The higher volume exchanges produced lower levels of residual solvents. In addition, lower residual solvents was measured for microparticles washed with ambient and warm water compared to those washed only with room temperature water. These results are consistent with general washing conditions observed with other microparticle formulations.

In vitro release at 24 hr was unaffected by the amount of volume exchanges during washing. With no hold time before washing, the burst release was higher for the warm water washing cycle. However, with a 200 min hold time before washing started, the in vitro 24 hr release was lower for the batches washed with warm water.

### Results

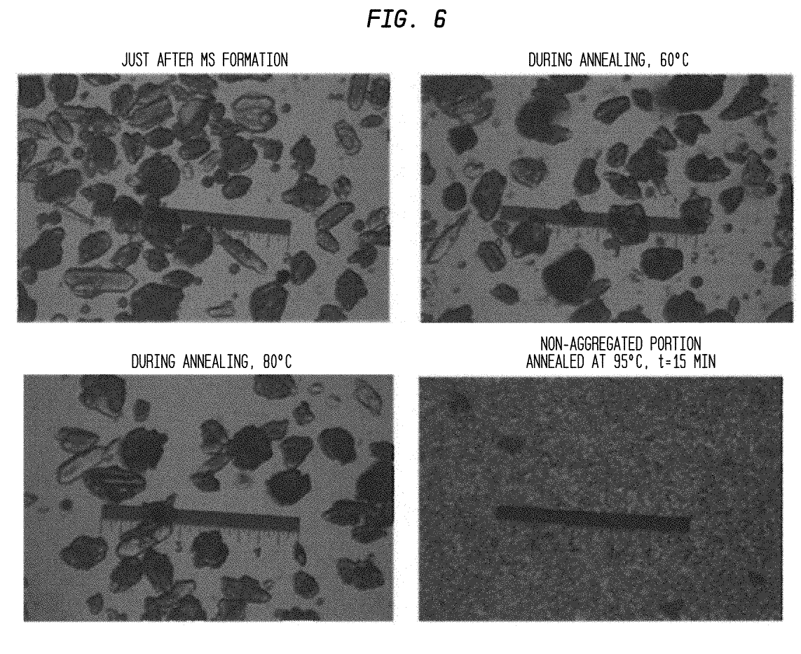

The release profiles of these prepared batches are shown in FIGS. 4-6.

The amount of washing volume exchanges had little effect on the in vitro release profiles (solid vs dashed lines), with all other parameters held constant.

The effect of washing temperature on the in vitro release profile is shown in FIG. 4. In general, warm water (dashed lines) washing slows the release rate.

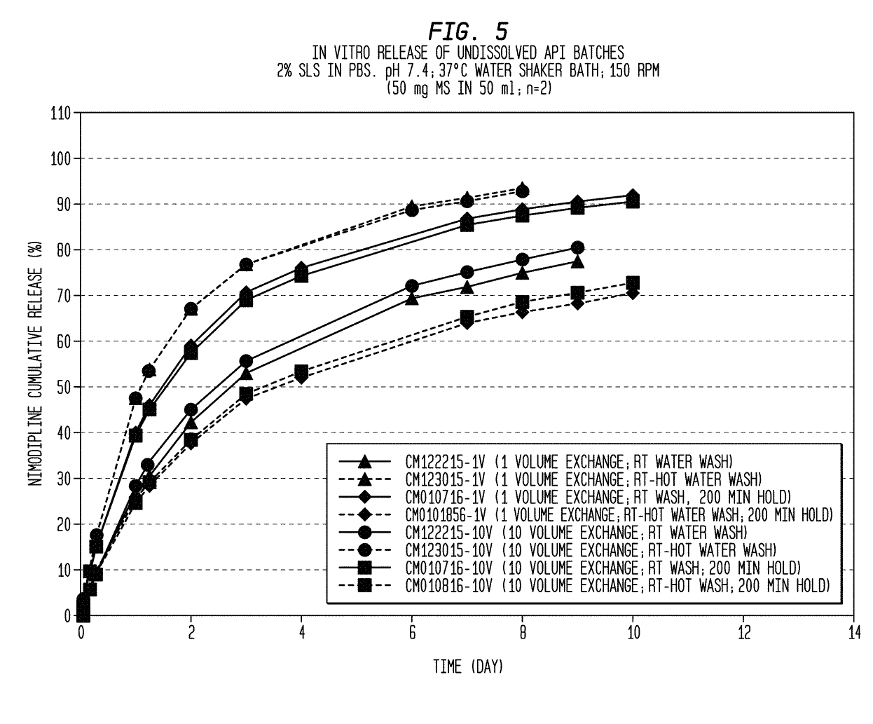

Finally, the effect of hold time is presented in FIG. 5. The introduction of a 200 minute hold time before washing (dashed lines) had varying effects on the in vitro release profile. It was discovered that the length of DP mixing time affects the drug crystal form, which in turn affects the in vitro release rate.

### Example 6. Polymorphic Form of Nimodipine

Nimodipine has two polymorphs, Form I (racemate) and Form II (conglomerate), sometimes referred to as modification I (Mod I) and modification II (Mod II). Mod I is the metastable polymorph, chemically defined as a racemate. It presents a higher solubility in water (0.036±0.007 mg 100 mL⁻¹′ at 25° C.) and a characteristic melting event at 124±1° C.) when compared to the stable polymorph Mod II, a conglomerate, which is less soluble in water (0.018±0.004 mg 100 mL⁻¹, at 25° C.) and melts at 116±1° C. Although Form II is the most stable polymorph at temperatures from 0 to 90° C., the drug powder is supplied as Form I (Manoela K. Riekes, et al, (2014) “Development and validation of an inherent dissolution method for nimodipine polymorphs,” Cent. Eur. J. Chem. 12(5): 549-56).

Nimodipine powder was milled to a specified size range and added to the polymer solution containing PLGA dissolved in ethyl acetate. Because nimodipine is above its solubility limit in the solvent, a supersaturated suspension is created whereby the drug is only partially solubilized in the ethyl acetate within the dispersed phase (DP).

Because Form II is the most stable, the conversion from Form I to Form II is initiated when the nimodipine is in contact with the solvent. X-ray diffraction methods can detect the presence of Form I, Form II and the amorphous state of the encapsulated nimodipine (see Riekes, M. K. et al, “Polymorphism in nimodipine raw materials: development and validation of a quantitative method through differential scanning calorimetry,” J. Pharmaceutical Biomed. Analysis 2012; 70: 188-93). For example, a reflection at 6.6° 28 was observed for Modification I, while that at 9.3° was present exclusively for Modification 2. Id. The polymorphs of nimodipine also can be distinguished by vibrational spectroscopy, although they exhibit basically identical Raman spectra characteristic of vibrations of the same molecule. Id. The peak intensities which characterize the C═C bond stretching of the dihydropyridine ring (at 1642 cm⁻¹) and the symmetric stretching vibrations of the —NO₂ group (at 1347 cm⁻¹) vary according to the crystal modification. Id. For Modification I, the peak at 1347 cm⁻¹ is more intense than that observed at 1642 cm⁻¹. Id. An inverse result is observed for Modification II. Id.

It was determined that the dispersed phase (DP) mixing time (0-60 min), the polymer concentration (14-30%) (i.e. the amount of ethyl acetate in the DP) and the filtration of the polymer solution may have a direct effect on the resulting polymorph of the nimodipine within the DP.

A series of DP solutions were prepared using the conditions shown in Table 8.

TABLE 8
Polymorph DP; variables

| | Variables | | |
|---|---|---|---|
| | Polymer | Hold time after | |
| Experiment | Concentration | dispersion complete | Polymer |
| # | (wt %) | (min) | Filtration |
| 1 | 14 | 0 | none |
| 2 | | 15 | none |
| 3 | | 60 | none |
| 4 | 22 | 0 | none |
| 5 | | 15 | none |
| 6 | | 60 | none |
| 7 | 22 | 0 | 0.2 μm |
| 8 | | 15 | 0.2 μm |
| 9 | | 60 | 0.2 μm |
| 10 | 30 | 0 | none |
| 11 | | 15 | none |
| 12 | | 60 | none |

At each hold time, the DP was sampled into a centrifuge tube, centrifuged for 15 minutes, removal of the supernatant (dissolved polymer and API in ethyl acetate), placed into freezer and then lyophilized. The polymorph present was determined under each condition. Under all conditions, the observed polymorph was a conglomerate, or Form II.

In addition, the supernatant of the “0” time point for each polymer concentration was dried and analyzed by XRPD. The supernatant contained dissolved polymer and dissolved nimodipine in ethyl acetate. All of these supernatant samples contained conglomerate (Form II) and amorphous nimodipine, the latter due to the dissolved portion of nimodipine.

The variables that affect the precipitation rate of the microparticle droplet and thus, the nimodipine polymorph within the microparticle, were determined to be CP/DP ratio and the amount of water dilution. In addition, the surfactant concentration, PVA, in the CP as well as the presence of ethyl acetate in the CO was varied using the normal CP/DP ratio of 80.

All of the conditions yielded the conglomerate (Form II) polymorph, and all but two of the samples contained amorphous nimodipine. In both of these cases, ethyl acetate was present in the CP and there was no water dilution added (PVA varied from 0.35 to 2%). These conditions would slow the precipitation of the microparticle/emulsion droplet due to the extra solvent in the CP and no water dilution.

### Methods to Increase the Release Rate of Nimodipine

It was apparent from previous determinations that microparticles containing Form I of nimodipine displayed a faster release than did microparticles containing Form II nimodipine. However, the conversion from Form I to Form II could not be prevented when the nimodipine was in contact with the ethyl acetate solvent. Only short DP mixing times could minimize this conversion, which will be difficult with a scaled-up batch process.

Several approaches to study polymorph conversion were evaluated, including temperature treatment, Form II API, reduction in crystalline size, and API Form I lot.

Effect of Temperature Treatment

An attempt was made to convert nimodipine Form II back to Form I using temperature. It has been shown in the literature that Form I has a characteristic melting point of 124±1° C. and Form II at 116±1° C. [Riekes, M K, et al., (2014), “Development and validation of an intrinsic dissolution method for nimodipine polymorphs,” Cent. Eur. J. Chewm. 12(5): 549-56] Two annealing studies were performed on a batch of microparticles that had completed the washing step. The suspension of microparticles in water was heated to ≈95° C. and held for 60 minutes. The first study used a lower molecular weight (28 kDa) poly(D-lactide-co-glycolide) polymer of inherent viscosity 0.24 dl/g, in which the lactide to glycolide mole ratio is 50:50, and the copolymer comprises an acid end group (Polymer A), while the second study used a higher molecular weight (44 kDa) polymer, poly(D-lactide-co-glycolide) polymer of inherent viscosity 0.38 dl/g, wherein the lactide to glycolide mole ratio is 50:50, and the copolymer comprises an acid end group (Polymer B). Table 9 shows the process parameters for both batches.

TABLE 9
Effect of temperature treatment; process parameters

| Lot# | CM011516 | CM012916 |
|---|---|---|
| Batch Size (g) | 30 | 30 |
| Polymer | A | B |
| Polymer IV (dl/g) | 0.24 dl/g | 0.38 dl/g |
| Polymer Molecular | 28 | 44 |
| Weight, M_{w} (kDa) | | |
| Target Drug | 65 | 65 |
| Load, (w/w) % | | |
| Nimodipine Lot | RD2277 Lusochimica | RD2277 Lusochimica |
| | lot 151313 | lot 151313 |
| DP Mixing Time | 65 | 66 |
| Dispersed Phase | 38 | 38 |
| Flow Rate, mL/min | | |
| Continuous Phase | 3 | 3 |
| Flow Rate, L/min | | |
| CP/DP ratio | 80 | 80 |
| Mixer Speed | 3000 | 3000 |
| Water Dilution | 1 | 1 |
| Flow Rate, L/min | | |
| Washing: Temp | 25° C. | 25° C. |
| & Duration | (10-15 volume exch.) | (10-15 volume exch.) |

The second study also analyzed in-process samples taken at 0° C., 60° C., 80° C. and 95° C. These final microparticles and in-process samples were characterized for drug load, polymer molecular weight, size, microscopy and in vitro release. The characterization results for these prepared batches and heat treatment are shown in Table 10.

TABLE 10
Effect of temperature treatment; characterization

| Parameter | CM011516 | | CM012916 |
|---|---|---|---|
| Batch Size (g) | 30 | | 30 |
| Polymer | A | | B |
| Polymer Inherent | 0.24 | | 0.38 |
| Viscosity (dl/g) | | | |
| Nimodipine Lot | RD2277 Lusochimica | | RD2277 Lusochimica |
| | lot 151313 | | lot 151313 |
| Mixer Speed (rpm) | 3000 | | 3000 |
| Drug (%) | 64.7 | | 65.4 |
| Particle size (μm) | initial | After 95° C. | |
| % < 10 | 21 | 5 | 60 |
| % < 25 | 55 | 11 | 93 |
| % < 50 | 88 | 32 | 125 |
| % < 75 | 120 | 91 | 154 |
| % < 90 | 155 | 128 | 178 |
| Residual Ethyl | 0.2 | Not | 0.2 |
| Acetate (wt %) | | detected | |

Results

For both studies, the polymer molecular weight decreased from the original polymer molecular weight during the heating step. This was most evident after the 1 hour hold at 95° C., where the drop was about 40% from the raw polymer. This drop in polymer molecular weight can be observed in the faster release profiles of the heat-treated microspheres (data not shown).

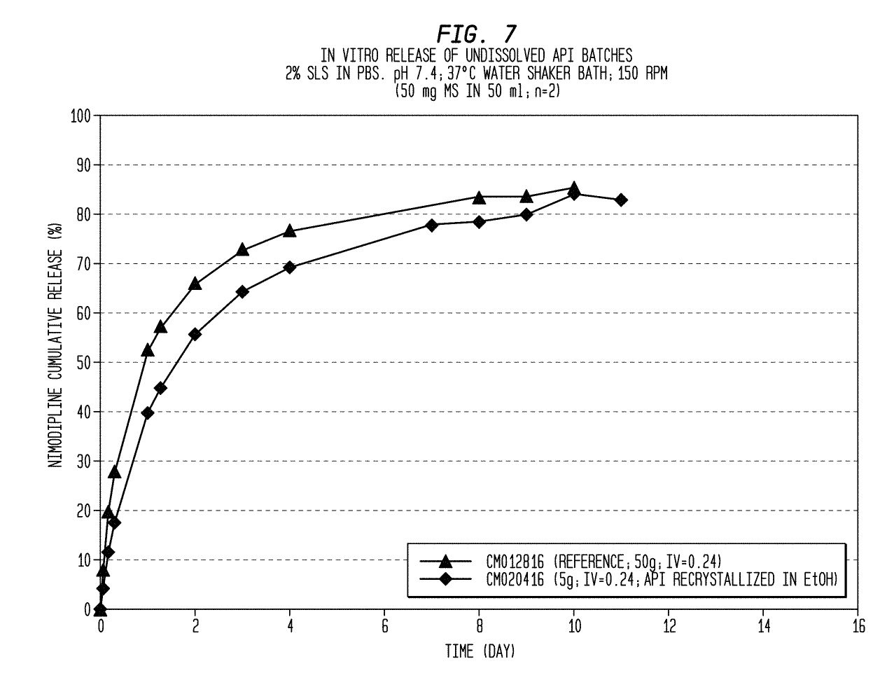

Light microscopy of lot CM012916 (FIG. 7) shows a gradual disintegration of the microspheres and drug crystals as the suspension temperature was increased, especially at the highest temperature.

The results showed that temperature treatment resulted in a gradual disintegration of the microparticles and drug crystals as the suspension temperature was increased, especially at the highest temperature. This approach to convert the encapsulated nimodipine back to Form I was not further pursued.

### Example 7. Preparation of Nimodipine Form II Loaded Microparticles from Form I nimodipine

A. Lab Scale

Five grams of polymorphic Form I nimodipine (UQUIFA 1092122001) were dissolved in ethanol, and then crystallized via a solvent evaporation process to yield form II, as described by Riekes, M., et al, “polymorphism in nimodipine raw materials: development and validation of a quantitative method through differential scanning calorimetry,” J. Pharmaceutical Biomedical Analysis (2012); 70: 188-193). In brief, a solution containing 500 mg of pure Modification 1 in 15 ml of ethanol was stirred until total solvent evaporation of the solvent at 298 K, resulting in an almost white powder, characteristic of pure Modification II. The samples were dried in an oven at 313±1 K.

The recrystallized drug powder was milled with a mortar and pestle to reduce the crystalline size.

A 5 gram lab-scale batch of the Form II nimodipine was used to prepare drug loaded microparticles with the process parameters shown in Table 11.

TABLE 11
Form II API; process parameters

| | CM012816 | |
|---|---|---|
| Lot# | (reference) | CM020416 |
| Batch Size (g) | 50 | 5 |
| Polymer | A | A |
| Polymer IV (dl/g) | 0.24 | 0.24 |
| Polymer Molecular | 28 | 28 |
| Weight, M_{w} (kDa) | | |
| Target Drug | 65 | 65 |
| Load, (w/w) % | | |
| Nimodipine Lot | 1092122001 | Recrystallized from |
| | | EtOH 1092122001 |
| DP Mixing Time | 60 | 71 |
| DP Treatment | None | none |
| Dispersed Phase | 75 | 75 |
| Flow Rate, mL/min | | |
| Continuous Phase | 3 | 3 |
| Flow Rate, L/min | | |
| CP/DP ratio | 40 | 40 |
| Mixer Speed | 3000 | 3000 |
| Water Dilution | 1 | 1 |
| Flow Rate, L/min | | |
| Washing: Temp | 25° C. | 25° C. |
| & Duration | (10-15 volume exch.) | (10-15 volume exch.) |

As a comparison, a reference lot CM012816, which started with nimodipine Form I, is also shown.

The characterization results for the Form II batch and the reference batch (CM020416) are shown in Table 12.

TABLE 12
Form II API; characterization

| | CM012816 | |
|---|---|---|
| Parameter | (reference) | CM020416 |
| Batch Size (g) | 50 | 5 |
| Polymer | A | A |
| Polymer Inherent | 0.24 | 0.24 |
| Viscosity (dl/g) | | |
| Nimodipine Lot | 1092122001 | Recrystallized from |
| | | EtOH 1092122001 |
| Drug (wt %) | 64.6 | 66.2 |
| Encapsulation | 99 | 102 |
| Efficiency (%) | | |
| Particle size (μm) | | |
| % < 10 | 41 | 32 |
| % < 25 | 62 | 51 |
| % < 50 | 86 | 72 |
| % < 75 | 110 | 97 |
| % < 90 | 130 | 125 |
| Residual Ethyl | 0.2 | 0.2 |
| Acetate (wt %) | | |

### Results

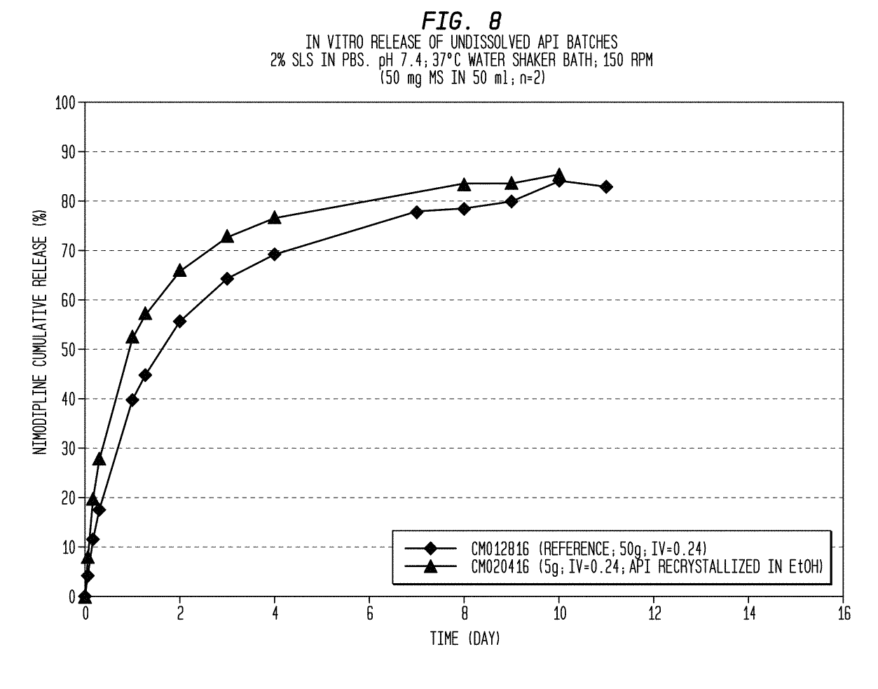

The in vitro release is shown in FIG. 8, with the reference batch shown as comparison. The particle size was smaller for the form II lot CM020416, and the in vitro release was faster compared to the reference material.

Formulation and Process Variables

The effect of the duration of the DP mixing step and the polymer molecular weight on microparticle characteristics, and the use of multi-compendial chemical components was analyzed.

Effect of Dispersed Phase Mixing Time

Previous work revealed that the extent of mixing time of the dispersed phase (DP) affected the characteristics of microparticles, especially the in vitro release profile. It was observed that the partially-dissolved nimodipine powder within the DP undergoes a polymorph transition from Form I to Form II. The extent of this transition appears, in part, to be dependent on the mixing time, before the microparticle formation step.

Small-scale batches (5 grams) were prepared using polymer A and R&D API lot (UQUIFA lot 10921328001). The effect of DP mixing time, 60 or 180 minutes, on the characteristics of the prepared microparticles was determined. Table 13 provides the formulation and process parameters for these two batches.

TABLE 13
Effect of DP mixing time; process parameters (5 g)

| Lot# | CM031416 | CM031516 |
|---|---|---|
| Batch Size (g) | 5 | 5 |
| Polymer | A | A |
| Polymer IV (dl/g) | 0.24 dl/g | 0.24 dl/g |
| Polymer Molecular | 28 | 28 |
| Weight, M_{w} (kDa) | | |
| Target Drug | 65 | 65 |
| Load, (w/w) % | | |
| Nimodipine Lot | 10921328001 | 10921328001 |
| DP Mixing Time | 60 | 180 |
| Dispersed Phase | 75 | 75 |
| Flow Rate, mL/min | | |
| Continuous Phase | 3 | 3 |
| Flow Rate, L/min | | |
| CP/DP ratio | 40 | 40 |
| Mixer Speed | 3300 | 3300 |
| Water Dilution | 4 | 4 |
| Flow Rate, L/min | | |
| Washing: Temp | 25° C. | 25° C. |
| & Duration | (10-15 volume exch.) | (10-15 volume exch.) |

All parameters were held constant except the DP mixing time. Compared to earlier small-scale batches, the DP flow rate was increased to 75 ml/min, which corresponded to a CP/DP=40. A higher CP/DP ratio allows for a faster total formation time step.

Results

The characterization of the two batches and effect of DP mixing time are shown in Table 14.

TABLE 14
Effect of DP mixing time; characterization (5 g)

| Parameter | CM031416 | CM031516 |
|---|---|---|
| Batch Size (g) | 5 | 5 |
| Polymer | 5050 DLG 2.5A | 5050 DLG 2.5A |
| | (Oakwood M-534-15-1; | (Oakwood M-534-15-1; |
| | Evonik LP-1004) | Evonik LP-1004) |
| Polymer Inherent | 0.24 | 0.24 |
| Viscosity (dl/g) | | |
| Nimodipine Lot | UQUIFA lot | UQUIFA lot |
| | 10921328001 | 10921328001 |
| DP Mixing Time | 60 | 180 |
| Drug (%) | 64.6 | 65.1 |
| Encapsulation | 99 | 100 |
| Efficiency (%) | | |
| Particle size (μm) | | |
| % < 10 | 34.1 | 28.2 |
| % < 25 | 55.1 | 52.3 |
| % < 50 | 77.5 | 79.9 |
| % < 75 | 99.7 | 108.4 |
| % < 90 | 121.1 | 136.9 |
| Residual Ethyl | 0.5 | 0.6 |
| Acetate (wt %) | | |

The drug load and encapsulation efficiency were very high and the residual solvent values were similar for both batches. The particle size distribution was slightly higher for the batch prepared with the longer DP mixing time (CM031516), probably due to the larger drug crystal size influencing the microsphere size.

The main difference is between the in vitro release profiles of the two batches, as shown in FIG. 9. The in vitro release of the longer DP mixing time batch (CM031516) is slower than the shorter mixing time batch (CM031416). This is most likely due to the greater conversion of Form I to Form II (and larger crystal size) as the DP mixing time is increased.

The effect of the DP mixing time also was analyzed at the 10× scale (i.e. 50 grams). Two batches were prepared identically, except for the DP mixing time (15 and 60 minutes), as shown in Table 15.

TABLE 15
Effect of DP mixing time; process parameters (50 g)

| Lot# | CM012716 | TR012816 |
|---|---|---|
| Batch Size (g) | 50 | 50 |
| Polymer | A | A |
| Polymer IV (dl/g) | 0.24 dl/g | 0.24 dl/g |
| Polymer Molecular | 28 | 28 |
| Weight, M_{w} (kDa) | | |
| Target Drug | 65 | 65 |
| Load, (w/w) % | | |
| Nimodipine Lot | UQUIFA lot | UQUIFA lot |
| | 10921220001 | 10921220001 |
| DP Mixing Time | 15 | 60 |
| Dispersed Phase | 75 | 75 |
| Flow Rate, mL/min | | |
| Continuous Phase | 3 | 3 |
| Flow Rate, L/min | | |
| CP/DP ratio | 40 | 40 |
| Mixer Speed | 3000 | 3000 |
| Water Dilution | 1 | 1 |
| Flow Rate, L/min | | |
| Washing: Temp | 25° C. | 25° C. |
| & Duration | (10-15 volume exch.) | (10-15 volume exch.) |

The characteristics of these two batches are shown in Table 16.

TABLE 16
Effect of DP mixing time; characterization (50 g)

| Parameter | CM012716 | TR012816 |
|---|---|---|
| Batch Size (g) | 50 | 50 |
| Polymer | A | A) |
| Polymer Inherent | 0.24 | 0.24 |
| Viscosity (dl/g) | | |
| Nimodipine Lot | UQUIFA lot | UQUIFA lot |
| | 10921220001 | 10921220001 |
| DP Mixing Time | 15 | 60 |
| Drug (%) | 64.5 | 64.6 |
| Encapsulation | 99 | 99 |
| Efficiency (%) | | |
| Particle size (μm) | | |
| % < 10 | 32 | 41 |
| % < 25 | 48 | 62 |
| % < 50 | 65 | 86 |
| % < 75 | 83 | 110 |
| % < 90 | 99 | 130 |
| Residual Ethyl | 0.2 | 0.2 |
| Acetate (wt %) | | |

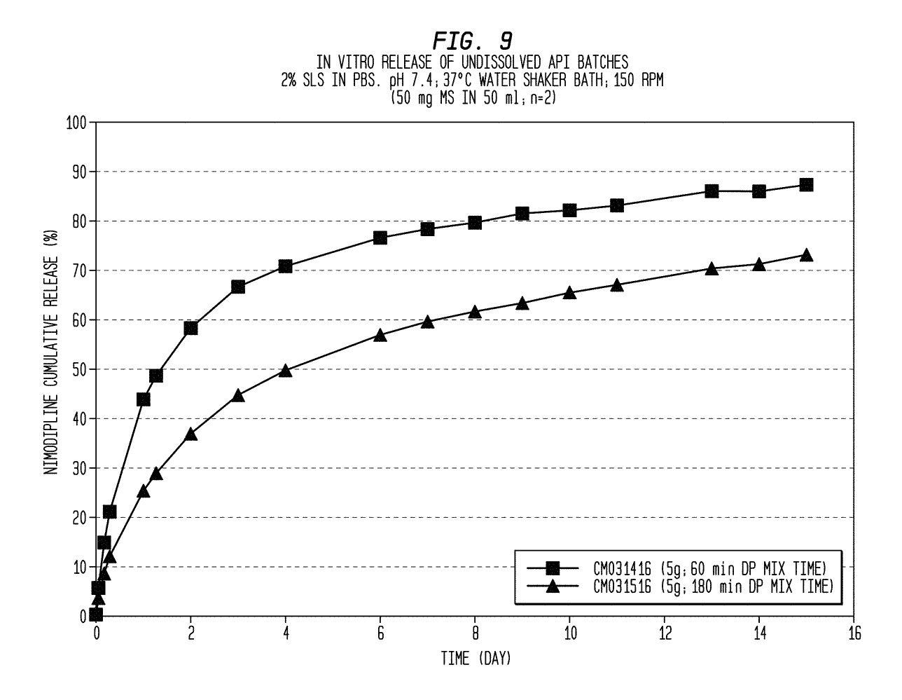

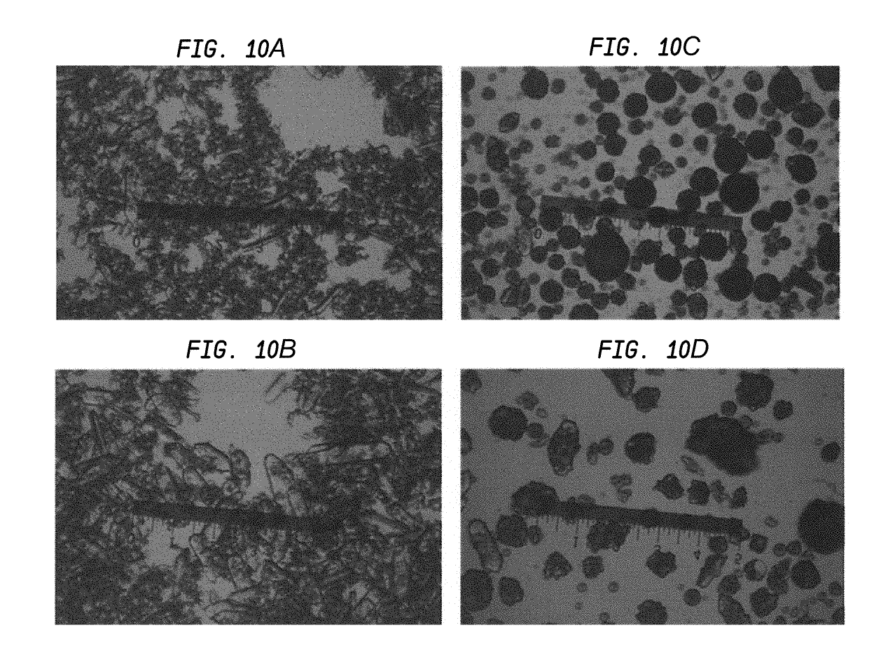

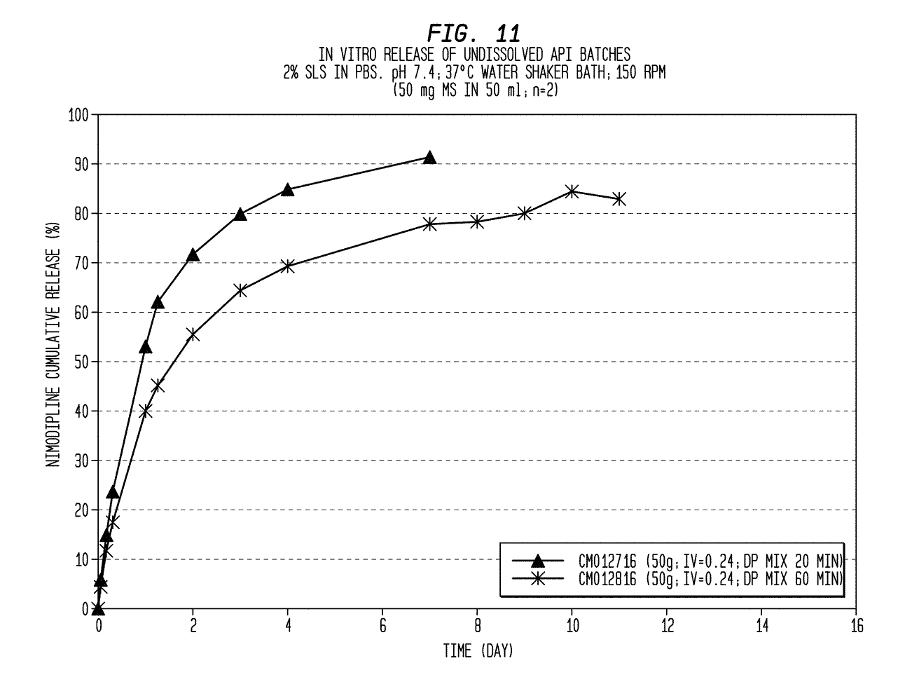

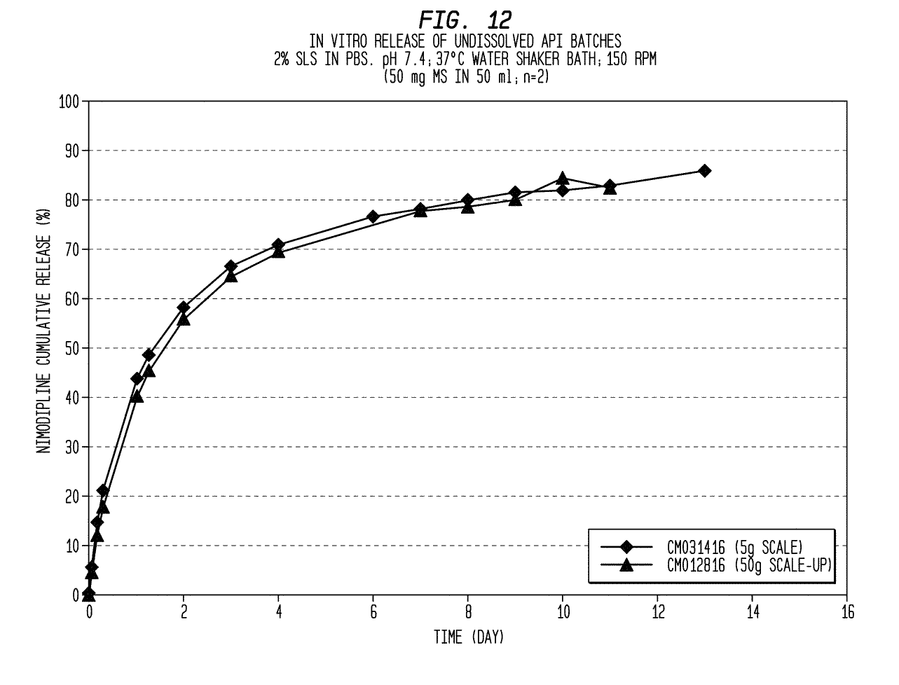

The microparticle size was larger for the long DP mixing time (lot TR012816) compared to the shorter mixing time (Lot CM012716). Again, this might be due to more conversion from Form I to Form II during the longer mixing time. FIG. 10 shows by light microscopy that at 15 minutes DP mixing, only a few large drug crystals are observed in the DP and the final washed microparticles (a and b). At 60 minutes, many large drug crystals can be seen in the DP and even in the microparticles (c and d). Thus, the DP mixing time has a significant effect on the extent of conversion from Form I to Form II. The longer the DP mixing time, the more Form II is formed.

The result of this conversion from polymorph Form I to polymorph Form II can be seen in the in vitro release profiles of these two batches as shown in FIG. 11. The batch prepared with the longer DP mixing time had an overall slower release profile compared to the 15 minutes DP mix time.

Effect of Polymer Molecular Weight

The effect of polymer molecular weight on the characteristics of the microparticles was determined at the 5 gram scale.

The results (data not shown) showed that the size of the microparticles is larger for the higher molecular weight polymer due to the higher viscosity of the DP solution (using the same homogenizer mixing speed). The in vitro release of the lower molecular weight polymer is slightly faster, as expected. However, since the particle size is also lower for CM012216 (i.e. larger surface area), the effect of polymer molecular weight may not have a significant impact for this product.

Effect of CP/DP Ratio

The ratio of continuous phase to the dispersed phase (CP/DP ratio) inside the mixing chamber determines the precipitation rate of the microparticle/emulsion droplet; and this value depends on the organic solvent used and its solubility in the aqueous medium. The CP/DP ratio can affect microparticle characteristics, such as drug load, size and release. The process described in FIG. 1 depends on a fast solidification of the microparticle in order for the hollow fiber filter (HFF) to operate efficiently.

Generally, the microparticle process uses a CP/DP ratio much higher than the solubility of the solvent (within the dispersed phase) in the aqueous continuous phase. To determine the effect of CP/DP ratio at the 5 gram scale, two batches were prepared with a CP/DP value of 10 and 80. The DP mixing time was similar between the two batches; the mixer speed and water dilution were also adjusted.

The results (data not shown) showed that drug load was slightly lower for the low CP/DP ratio batch (CM011316). The size distribution is different between the two batches; however this is most likely due to the different mixer speed. The in vitro release profiles for these two batches displayed similar curves.

Thus, CP/DP ratio in the range of 10-80 had some effect on microparticle characteristics, but not on in vitro release.

Multi-Compendial Raw Materials

Previous batches used a non-compendial grade of polyvinyl alcohol (PVA) in the continuous phase (CP) and NF grade of ethyl acetate in the dispersed phase (DP). To proceed to scaled-up and GMP batches, it was necessary to source multi-compendial materials for the manufacture of nimodipine microparticles.

A small-scale batch was made with multi-compendial PVA (CM031116) and multi-compendial ethyl acetate (CM031016) to determine if there was any effect on the microparticle characteristics. The results (data not shown) indicate that the multi-comdendial source of PVA and ethyl acetate have no effect on microparticle characteristics and can be used for scale-up and GMP manufacturing.

### Example 8. Scale Up to 50 g

Lot CM031416 was scaled up to 50 grams. Several differences between the batches included the API lot, mixer speed and water dilution flow rate, (reference 5 g scale, CM031416). Characteristics of the 5 g and 50 g scales are shown in Table 17.

TABLE 17
Scale-up to 50 g; characterization

| Parameter | CM031416 | TR012816 |
|---|---|---|
| Batch Size (g) | 5 | 50 |
| Polymer | A | A |
| Nimodipine Lot | lot 10921328001 | lot 10921220001 |
| Drug (%) | 64.6 | 65.8 |
| Encapsulation | 99 | 100 |
| Efficiency (%) | | |
| Particle size (μm) | | |
| % < 10 | 34.1 | 33.9 |
| % < 25 | 55.1 | 54.0 |
| % < 50 | 77.5 | 76.4 |
| % < 75 | 99.7 | 100.5 |
| % < 90 | 121.1 | 125.7 |
| Residual Ethyl | 0.5 | 0.5 |
| Acetate (wt %) | | |

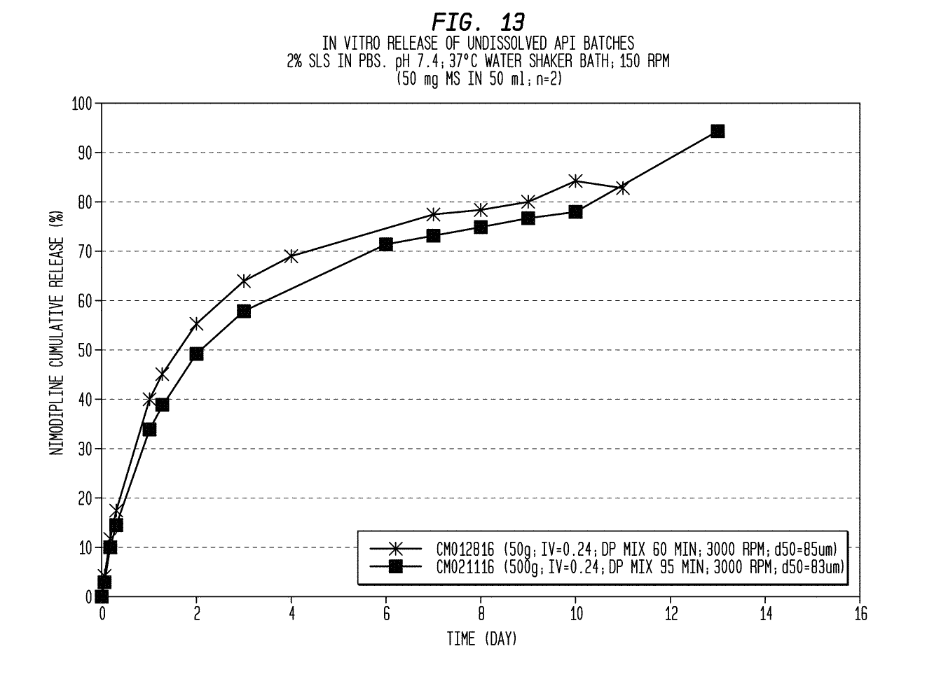

The in vitro release profiles of the 5 gm and 50 gm scales are shown in FIG. 12. Similar characteristics and release profiles show that scaling up from 5 to 50 grams had little effect.

### Example 9. Scale Up to 500 Grams

The 50 gram batch, CM012816, was scaled up to 500 grams, maintaining the processing parameters as close as possible. The water dilution rate was increased from 1 to 3 L/min to help control the solvent effect during 10× scaling. The polymer solution was filtered (to mimic GMP conditions) and the DP mixing time was longer for the 500 g batch.

Part of the 500 g batch was filled into 20 ml vials using a 10 g suspension fill. The vials and bulk microspheres were then lyophilized to remove the water content. The resulting properties of the microspheres are shown in Table 18.

TABLE 18
Scale up to 500 g: Characterization.

| | | CM012816 | |
|---|---|---|---|
| | Parameter | (reference) | CM021116 |
| | Batch Size (g) | 50 | 500 |
| Preparation | Polymer | A | A |
| Parameters | Polymer Lot | M-534-15-1 | M-534-15-1 |
| | Polymer | 0.24 | 0.24 |
| | Inherent | | |
| | Viscosity (dl/g) | | |
| | Nimodipine Lot | lot | lot |
| | | 10921220001 | 10921220001 |
| | Polymer | None | PTFE |
| | Solution | | (Sartifluor 150 |
| | Filtration | | Capsule; |
| | | | Sartorious) |
| | Mixer Speed | 3000 | 3000 |
| | (rpm) | | |
| Properties | Drug load in | 64.6 ± 0.35 | 64.4 ± 0.19 |
| of Bulk | the Bulk MS | | |
| NIM-MS | (%) | | |
| | Particle size (μm) | | |
| | % < 10 | 41 | 38 |
| | % < 25 | 62 | 60 |
| | % < 50 | 86 | 83 |
| | % < 75 | 110 | 106 |
| | % < 90 | 130 | 126 |
| Properties of | Moisture | N/A | 0.11 ± 0.02 |
| Vialed NIM-MS | Content (%) | | |
| | Residual Ethyl | N/A | 0.3 |
| | Acetate (wt %) | | |

The drug load and particle distribution of the 500 g batch was very similar to that of the 50 gram batch. The in vitro release profile for the 50 g batch and for the 500 g batch is shown in FIG. 13. The release from the scaled up batch is slightly slower than the 50 g batch, possibly due to increased solvent exposure of the microspheres during the formation step.

### Example 10. Scale Up to 2 kg

The polymer powder was added to the stirred solvent using a top-stirring glass vessel. The water dilution rate was increased to 4 L/min to minimize any solvent exposure during the formation step. DP mixing time was 67 minutes for this batch. For scaled up batches, the washed microspheres were sieved using a 250 μm sieve bag to remove any large or agglomerated microspheres and ensure syringeability in the finished product vials. The extent of washing was increased to 25 volume exchanges in order to remove any residual PVA within the suspension. Batch CM030216 had no issues during the formation, washing, sieving and filling steps of the process.

The results (data not shown) showed that drug load and residual solvent content for this batch were on target. Similar to the smaller scale batches, the molecular weight is not affected by the microsphere encapsulation process. The microsphere size and distribution for this batch was larger than the 50 and 500 gram scales.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A pharmaceutical composition formulated for delivery by injection containing a microparticulate formulation comprising
(a) a suspension of microparticles comprising a therapeutic amount of a substantially pure Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide) polymer matrix, and
(b) a pharmaceutically acceptable carrier comprising an agent that affects viscosity of the microparticulate suspension,
wherein the microparticulate suspension comprising the polymorphic Form II of nimodipine is light stable, the Polymorphic form II of nimodipine is chemically stable, release profile is consistent from batch-to-batch, and particle size is controllable.

2. The pharmaceutical composition according to claim 1, wherein
(a) the microparticulate suspension comprises a plurality of microparticles; or
(b) the microparticles are of a uniform distribution of microparticle size; or
(c) the mean particle size (D50) of the microparticles ranges from 20 μm to 250 μm; or
(d) the concentration of the polymer ranges from about 14% to about 30%; or
(e) the lactide to glycolide ratio of the poly (lactide-co-glycolide) is 50:50; or
(f) inherent viscosity of the polymer is at least 0.16 dl/g; or
(g) molecular weight of the polymer is at least 28 kDa; or
(h) the polymorphic Form II of nimodipine is dispersed throughout the polymer matrix; or
(i) the polymer matrix is impregnated with the polymorphic Form II of nimodipine; or
(j) percentage of nimodipine retained by the microparticles relative to the total amount available is about 95%; or
(k) the microparticulate suspension is characterized by a drug load of about 65% polymorphic Form II of nimodipine by weight relative to the total weight of the formulation.

3. The pharmaceutical composition according to claim 1, wherein
(a) the polymorphic Form II of nimodipine includes less than 20% by weight of any other physical forms of nimodipine; or
(b) the microparticulate formulation contains less than 10% polymorphic Form I of nimodipine; or
(c) the microparticulate formulation is substantially free of polymorphic Form I of nimodipine.

4. The pharmaceutical composition according to claim 1, wherein the suspension of microparticles comprising a therapeutic amount of the polymorphic Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide)polymer matrix is prepared by a scalable process comprising:
(a) providing an API starting material containing a substantially pure polymorphic Form I of nimodipine;
(b) forming polymorphic Form II of nimodipine in situ by (i) adding the API starting material of (a) to a polymer solution, and (ii) creating a mixture of the polymorphic Form II of nimodipine and the polymer solution;
(c) homogenizing the mixture of (b) to form a disperse phase comprising the nimodipine;
(d) providing a continuous phase in which the dispersed phase will form an emulsion;
(e) introducing the dispersed phase and continuous phase into a reactor vessel, the reactor vessel including a continuous process medium, and forming an emulsion of the dispersed phase in the continuous phase comprising the nimodipine;
(f) causing the polymer to form microparticles containing polymorphic Form II of nimodipine;
(g) transporting the emulsion from the reactor vessel to a solvent removal vessel and removing the solvent;
(h) formulating the nimodipine Form II-containing microparticles by: (i) maintaining a suspension of nimodipine Form II-containing microparticles in the continuous phase; and (ii) washing the nimodipine Form II-containing microparticles; and
(i) drying the nimodipine Form II-containing microparticles.

5. The pharmaceutical composition prepared by the process according to claim 4, wherein:
(a) the API starting material is milled or unmilled;
(b) the solvent comprises ethyl acetate; and
(c) the washing is conducted by
(i) replacing the continuous phase with water by moving the suspension through a filter adapted to remove continuous phase and return the microparticles to a process vessel while maintaining the suspension;
(ii) replacing the ethyl acetate with water by moving the suspension through a filter adapted to eliminate the ethyl acetate and return the microparticles to a process vessel while maintaining the microparticles in suspension; and
(iii) removing the suspension of microparticles containing the bioactive agent and formulating medium from the process vessel; or
the washing is conducted by moving the suspension through a hollow fiber filter.

6. The pharmaceutical composition prepared by the process according to claim 4, wherein in step (i) the drying is by lyophilization or by a vacuum dryer.

7. The pharmaceutical composition prepared by the process according to claim 4, wherein the distribution of microparticle size is such that D10>20 μm, D50 is 70-80 μm, and D90 is <200 μm.

8. The pharmaceutical composition according to claim 1, wherein the suspension of microparticles comprising a therapeutic amount of the polymorphic Form II of nimodipine that has an X-ray powder diffraction (XRPD) spectrum substantially the same as the X-ray powder diffraction (XRPD) spectrum shown in FIG. 14B, a melting temperature of 116±1° C. as measured by differential scanning calorimetry, or both in a poly(lactide-co-glycolide) polymer matrix is prepared by a scalable process comprising:
(1) preparing an API starting material containing a substantially pure polymorphic nimodipine Form II by:
(a) synthesizing an API starting material containing substantially pure polymorphic Form II of nimodipine; or
(b) crystallizing Form II of nimodipine from Form I by dissolving Form I of nimodipine in a first solvent and evaporating the first solvent to yield Form II;
(2) completing the disperse phase by adding the API starting material of step (1) to a polymer solution, thereby creating a mixture of polymorphic Form II of nimodipine and the polymer solution in a second solvent;
(3) homogenizing the continuous phase comprising polyvinyl alcohol (PVA) in water with the dispersed phase of step (2) to form an emulsion;
(4) introducing a water stream continuously post-microparticle formation, causing the polymer to form nimodipine Form II-containing microparticles;
(5) transporting the emulsion from the reactor vessel to a solvent removal vessel and removing the solvent;
(6) formulating the Form II containing microparticles by
(i) maintaining a suspension of the Form II containing microparticles in the continuous phase;
(ii) washing the Form II containing microparticles; and
(7) drying the Form II containing microparticles.

9. The pharmaceutical composition prepared by the process according to claim 8, further comprising milling, micronizing or both the API starting material.

10. The pharmaceutical composition prepared by the process according to claim 9, wherein the API starting material containing the substantially pure polymorphic form II of nimodipine is characterized by a distribution of particle size of D10>2μ, D50>7μ and D90<10 μm.

11. The pharmaceutical composition prepared by the process according to claim 8, wherein
(a) the first solvent is ethanol;
(b) the second solvent is ethyl acetate; and
(c) the washing is conducted by
(i) replacing the continuous phase with water by moving the suspension through a filter adapted to remove continuous phase and return the microparticles to a process vessel while maintaining the suspension;
(ii) replacing the ethyl acetate with water by moving the suspension through a filter adapted to eliminate the ethyl acetate and return the microparticles to a process vessel while maintaining the microparticles in suspension; and
(iii) removing the suspension of microparticles containing the bioactive agent and formulating medium from the process vessel; or
the washing is conducted by moving the suspension through a hollow fiber filter.

12. A method for reducing severity or incidence of a delayed complication associated with a brain injury including interruption of a cerebral artery that deposits blood in a subarachnoid space, wherein the delayed complication is selected from the group consisting of a microthromboembolism, a delayed cerebral ischemia (DCI) caused by formation one or more of microthromboemboli, or cortical spreading ischemia (CSI) and a cortical spreading ischemia (CSI) comprising:
a) providing the pharmaceutical composition according to claim 1, and
(b) administering the pharmaceutical composition locally, either
(i) intraventricularly;
(ii) intracisternally into the subarachnoid space in a subarachnoid cistern;
or
(iii) intrathecally into the spinal subarachnoid space,
wherein the therapeutic amount of the substantially pure polymorphic Form II of Nimodipine having an X-ray powder diffraction spectrum substantially the same as the X-ray powder diffraction (XRPD) spectrum shown in FIG. 14B, a melting point of 116±1° C. as measured by differential scanning calorimetry or both that contacts and flows around the at least one cerebral artery in the subarachnoid space is effective to improve cerebral perfusion and to treat the delayed complication without entering systemic circulation in an amount to cause unwanted side effects including systemic hypotension and pulmonary vasodilation with pulmonary edema.
